(19) United States
(12) Patent Application Publication
(10) Pub. No.: US 2012/0054914 A1
(43) Pub. Date: Mar. 1, 2012

(54) HIGH STARCH ACCUMULATION IN PLANTS

(75) Inventors: Thomas D. Sharkey, East Lansing, MI (US); Sean Weise, East Lansing, MI (US)

(73) Assignee: BOARD OF TRUSTEES OF MICHIGAN STATE UNIVERSITY

(21) Appl. No.: 12/880,988

(22) Filed: Sep. 13, 2010

Related U.S. Application Data
(60) Provisional application No. 61/241,452, filed on Sep. 11, 2009.

(51) Int. Cl. A01H 1/06; A23K 1/00; C10L 1/00; A01H 5/00

(52) U.S. Cl. 800/284; 800/298; 426/635; 44/307

(57) ABSTRACT

Described herein are plants with increased starch accumulation without the corresponding loss in growth and/or yield.

### PRIORITY OF INVENTION

This application claims the benefit of priority under 35 U.S.C. §119(e) to U.S. Provisional Patent Application Ser. No. 61/241,452, filed Sep. 11, 2009, which application is hereby incorporated by reference in its entirety.

### STATEMENT OF GOVERNMENT RIGHTS

This work was funded by the Great Lakes Bioenergy Research Center, sponsored by the United States Department of Energy Grant No. 61-5751-1.2. The government has certain rights to this invention.

### BACKGROUND OF THE INVENTION

Green tissues of plants, mostly leaves and stems, use the process of photosynthesis to convert carbon dioxide and water to sugars for use as metabolic energy and to build plant structures. These green tissues also convert the sugars into starch to be used as energy stores. In most plants, including crops such as corn, starch accumulated during the day is usually broken down each night resulting in very little starch accumulation in the leaves; rather, these sugars are transported to the developing seeds or grain where they are deposited as starch to be used by the developing seedlings.

Starch is highly significant to man. For example, it forms a major component of animal diets, supplying man and domestic animals with a large portion of their carbohydrate intake. Starch is also used industrially in the production of paper, textiles, plastics and adhesives, as well as providing the raw material for some bioreactors that, for example, generate biofuel. The quantity of starch present in the harvested organ of a plant (e.g., leaves) will affect the gross yield and the processing efficiency.

Plant materials including grains and green-tissues are primary animal feed ingredients. In particular, corn silage, the feed-stuff derived from the anaerobic fermentation of maize forage, is a major dietary component of ruminant feed, especially for dairy production. Silage is abundantly available and composed of a large proportion of slowly digestible cellulosic material. However, silage-based diets are supplemented with various grains to provide rapidly digestible starch.

Biofuel can be defined as renewable energy source derived from agricultural feedstocks. There are two main types of biofuel: biodiesel and bio-ethanol (and other bio-based alcohols). Biodiesel is produced through chemical modification of plant oils produced from oil-crops such as canola, soya, peanuts or palms, or alternatively from waste “cooking oils” obtained from the food industry. Bio-ethanol is produced by microbial fermentation of plant-based carbohydrates, including sucrose, from sugarcane; starch, mostly from corn grain harvested as an energy crop; and from cellulosic materials (primarily plant cell wall material), mostly crop residues such as corn stover (waste leaves and stems) or dedicated cellulosic crops such as switchgrass or poplars.

### SUMMARY OF THE INVENTION

The invention focuses on increasing the yield of easily degraded polymers, such as starch, in plants, including leaves. The goal is to make leaves better for silage used to feed, for example, dairy cattle and to produce a better feedstock for a cellulosic ethanol fermentor or fermentors that make other bio-based products. The approach discussed herein provides for (1) increased leaf starch content in silage, eliminating the need to incorporate grain, (2) higher yield, and (3) ease and completeness of fermentation. The approach discussed herein overcomes the issue of reduced yield that is seen in most starch accumulating plants.

To overcome the yield penalty, one embodiment blocks starch degradation at a developmental time point late in the life cycle of the plant. This is different from already existing high starch plants because pre-existing high starch plants accumulate, and are unable to degrade, their starch throughout their life—i.e. they are “born” with high starch amounts in their leaves. Since currently existing high starch plants cannot degrade their starch early in their life, they do not grow as fast as plants that can degrade their starch and thus result in reduced yields.

One embodiment provides a method to accumulate starch in a plant without greatly decreasing plant growth or yield comprising inhibiting expression of a protein or a gene coding for a protein that aids in starch degradation, wherein expression of the protein or the gene coding for the protein that aids in starch degradation is inhibited by expression of a heterologous polynucleotide in the plant so as to accumulate starch in a plant without a significant decrease in yield or plant growth. In one embodiment, the expression is inhibited after grain development or prior to (such as just prior/immediately before (for example, after the plant has had a chance to reach maturity or about full, adult height and/or mass) or at senescence. In one embodiment the heterologous polynucleotide codes for an RNA sequence which upon expression in the plant leads to a reduction in expression of the protein or a gene coding for the protein that aids in starch degradation. In another embodiment, the heterologous polynucleotide comprises an inducible promoter or a promoter that drives expression prior to or at senescence and is not a constitutive promoter. In one embodiment, the promoter is an alcohol-inducible promoter or a senescence-induced promoter. In one embodiment, the protein is glucan water dikinase, phosphoglucan phosphatase or β-amylase.

In one embodiment, the expression is inhibited by RNA interference (RNAi). In another embodiment, the heterologous polynucleotide comprises a nucleotide sequence that is antisense to at least a portion of a nucleotide sequence that codes for the protein that aids in starch degradation.

In one embodiment, the increased starch accumulation is in the leaves of the plant. In one embodiment the plant is a monocot, such as maize, switchgrass, or miscanthus. In another embodiment, the plant is a dicot, such as tobacco, potato, cabbage, soybean or sweet potato. In one embodiment the plant is Arabidopsis.

Another embodiment provides a method for preparing a plant that accumulates starch without decreasing plant growth or yield comprising introducing into the plant a heterologous polynucleotide that encodes an RNA sequence which upon expression in a plant leads to a reduction in expression of a protein or a gene coding for a protein that aids in starch degradation, wherein the polynucleotide is integrated into the genome of a plant cell and an intact plant is generated from the plant cell and reduction in expression of the protein or the gene coding for the protein that aids in starch degradation occurs without a significant decrease in yield or plant growth. In one embodiment, the expression is inhibited after grain development or prior to (such as just prior/immediately before) or at senescence. In one embodiment the heterologous polynucleotide comprises an inducible promoter or a promoter that drives expression at senescence and is not a constitutive promoter. In another embodiment the promoter is an alcohol-inducible promoter or a senescence-induced promoter. In one embodiment, the protein is glucan water dikinase, phosphoglucan phosphatase or β-amylase.

In one embodiment, expression is inhibited by RNA interference (RNAi). In another embodiment, the heterologous polynucleotide comprises a nucleotide sequence that is antisense to at least a portion of a nucleotide sequence that codes for a protein that aids in starch degradation.

In one embodiment, the increased starch accumulation is in the leaves of the plant. One embodiment provides that the plant is a monocot, including maize, switchgrass, or miscanthus. Another embodiment provides that the plant is a dicot, such as tobacco, potato, cabbage, soybean or sweet potato. In one embodiment, the plant is Arabidopsis.

On embodiment provides a transgenic plant or plant material that comprises a heterologous polynucleotide that encodes an RNA sequence which upon expression in a plant leads to a reduction in expression of a protein or a gene coding for a protein that aids in starch degradation, wherein reduction in expression of the protein or the gene coding for the protein that aids in starch degradation occurs without a significant decrease in yield or plant growth. In one embodiment, the expression is inhibited after grain development or prior to (such as just prior/immediately before) or at senescence. In one embodiment, the heterologous polynucleotide comprises an inducible promoter or a promoter that drives expression at senescence and is not a constitutive promoter. In one embodiment, the promoter is an alcohol-inducible promoter or a senescence-induced promoter. In one embodiment, the protein is glucan water dikinase, phosphoglucan phosphatase or β-amylase.

In one embodiment, expression is inhibited by RNA interference (RNAi). In another embodiment, the heterologous polynucleotide comprises a nucleotide sequence that is antisense to at least a portion of a nucleotide sequence that codes for a protein that aids in starch degradation.

In one embodiment, the increased starch accumulation is in the leaves of the plant. In one embodiment, plant is a monocot or said plant material is from a monocot plant, such as maize, switchgrass, or miscanthus. In one embodiment, the plant is a dicot or said plant material is from a dicot plant, such as tobacco, potato, cabbage, soybean or sweet potato. In one embodiment, the plant is Arabidopsis. In one embodiment, the plant material is selected from plant tissue, plant cells, plant seeds or protoplasts.

One embodiment provides a method to prepare animal feed silage comprising producing animal feed silage from the transgenic plant or plant material described herein. Another embodiment provides a method to prepare bio-fuel comprising producing biofuel from the transgenic plant or plant material described herein. In one embodiment, the biofuel is bio-ethanol.

One embodiment provides a method to accumulate starch in a plant without decreasing plant growth or yield comprising inhibiting endogenous expression of a protein or a gene coding for a protein that aids in starch degradation throughout the life of the plant, wherein the plant is complimented with a transgene coding for the protein which was inhibited which is operably linked to a promoter which is only active early in the life cycle of the plant so as to accumulate starch in the plant without a significant decrease in yield or plant growth. In one embodiment, the endogenous expression of a protein or a gene coding for a protein that aids in starch degradation is inhibited by gene knock-out through gene mutation, tDNA, antisense technology, or RNAi. In one embodiment, the protein is glucan water dikinase, phosphoglucan phosphatase or β-amylase. In one embodiment, the increased starch accumulation is in the leaves of the plant. In one embodiment, the plant is a monocot, such as maize, switchgrass, or miscanthus. In another embodiment, the plant is a dicot, such as tobacco, potato, cabbage, soybean or potato (including sweet potato). In one embodiment the plant is Arabidopsis, while in another embodiment, the plant is Zea mays. One embodiment provides for a method for preparing a plant that accumulates starch without decreasing plant growth or yield as described herein; another embodiment comprise a transgenic plant or plant material. One embodiment provides a method to prepare animal feed silage or bio-fuel comprising producing animal feed silage or bio-fuel (e.g., ethanol) from the transgenic plant or plant material.

### BRIEF DESCRIPTION OF THE DRAWINGS

FIGS. 1A-B. Senescence-inducible and Alcohol-inducible RNAi Constructs: (A) The promoter of the SAG12 gene, a cysteine protease (Yoo-Sun Noh and Rick Amasino (1999)), is active only at or near senescence in the plant Arabidopsis and was used to drive RNAi targeted to the glucan water dikinase enzyme (GWD enzyme) or the phosphoglucan phosphatase enzyme. (B) An alcohol-inducible promoter was used to drive RNAi. The advantage of an inducible promoter, such as the alcohol-inducible promoter, is that the developmental timing of starch accumulation can be precisely controlled.

FIGS. 2A-B. A) Senescence Induced RNAi Starch Accumulating plants: Photographs were taken and leaves were stained with iodine for starch in the morning when starch levels are the lowest. The plants that are lacking the glucan water dikinase enzyme (GWD KO) have high starch levels throughout their life and a significant growth penalty. The plant with the senescence-induced RNAi gene against GWD does not exhibit a decrease in growth after 2 months and contains high leaf starch levels comparable to the GWD KO line. B) Lines A through D have the SAG12 driven RNAi and can be compared to the WT. The data show that at week 5 there was no excess starch, but by week 13 there was much more starch at the end of the night in four different SAG lines than in the WT.

FIG. 3. Alcohol Induced RNAi Starch Accumulation: Wild type, primary transformant plants containing the alcohol inducible RNAi against the glucan water dikinase (GWD) or the phosphoglucan phosphatase enzyme, both involved in leaf starch degradation, and the corresponding knock out lines (plants that lack enzyme throughout their lives) were grown for 27 days. Alcohol inducible plants were then sprayed with 2% ethanol. Leaves were taken for starch determinations in the morning. The alcohol inducible plants quickly accumulate as much starch as the knock out line. In the case of the Alc inducible RNAi against the phosphoglucan phosphatase, the starch levels exceeded those of the knock out line.

FIG. 4 provides sequences of RNAi inverted repeats against Arabidopsis genes SEX1 and SEX4 (SEQ ID NOs: 1 and 2).

FIG. 5 provides examples of enzymes that can be blocked to cause leaf starch accumulation. They are listed by protein name, gene name, and Arabidopsis gene number (SEQ ID NOs: 3-20).

FIG. 6 provides examples of promoters that can be used to cause starch accumulation at or near senescence. They are listed by protein name, gene name, and Arabidopsis gene number (SEQ ID NOs: 21-33).

FIG. 7 provides starch levels in WT, azygous, and alcohol inducible empty vector control lines with and without alcohol. Plant material was harvested just before the lights came on in the growth chamber when starch levels are at a minimum. Values are mean±SE (n=5).

FIG. 8 provides starch levels in alcohol inducible RNAi lines against the gene for glucan water dikinase or phosphoglucan phosphatase. Plant material was harvested just before the lights came on in the growth chamber when starch levels are at a minimum. Values are mean±SE (n=5).

FIG. 9 provides transcript levels for GWD and PGP in their respective RNAi line relative to the transcript level for the Actin2 gene. Plant material was harvested just after the lights went off in the growth chamber when GWD and PGP transcript level have been shown to be at their peak. Values are mean±SE (n=5).

FIG. 10 provides alcohol induced RNAi starch accumulation in Arabidopsis. Azygous control lines (squares) and two transgenic RNAi lines against the gene for GWD; line C (triangles) and line E (circles). Plants were sprayed daily with 3% ethanol immediately following the initial starch measurement made at week six. Values are mean±SE (n=5).

FIG. 11 provides total above ground dry biomass during the exponential phase of growth. Azygous control lines (squares), two transgenic RNAi lines against the gene for GWD; line C (triangles) and line E (circles), and a GWD KO line (diamonds) were sprayed daily with 3% ethanol immediately following the initial biomass measurement made at week three. Values are mean±SE (n=6).

FIG. 12 provides starch levels in transgenic maize RNAi lines against the SEX1 like gene. Plant material was harvested just before the lights came on in the growth chamber when starch levels are at a minimum in control lines. Values are mean±SE (n=5).

FIG. 13 provides transcript levels for the SEX1 like gene in maize relative to the transcript level for the α-actin gene. Plant material was harvested just after the lights went off in the growth chamber. Values are mean±SE (n=4).

FIG. 14 provides the leaf cross section of maize RNAi line against the SEX1 like gene. Starch was stained a brown color using quarter strength Lugol solution. Empty pockets in section are due to tearing of section as a result of older leaf material used. Bar=50 μm.

FIG. 15 provides total above ground biomass of transgenic maize lines. One representative plant from each line was chosen at random for harvest. Plants had started to flower at time of harvest. (n=1).

### DETAILED DESCRIPTION OF THE INVENTION

Unless defined otherwise, all technical and scientific terms used herein have the same meaning as commonly understood by one of ordinary skill in the art to which this invention belongs. The materials, methods and examples are illustrative only and not limiting. The following is presented by way of illustration and is not intended to limit the scope of the invention.

Ethanol is a popular proposed alternative to gasoline. Generally, ethanol is made from sugars derived from grain. Making ethanol is an expensive process and increases the price of grain. The largest stores of sugars in plants are generally in the body of the plant itself in the form of cellulose and starch. There is currently great interest in using the body of the plant (plant biomass) instead of grain to produce ethanol.

Thus, the present invention provides for the production of plants with high amounts of leaf starch and their use in bio-ethanol or other biofuel production or as animal feed (e.g., as a silage or silage-type feed for animals, such as ruminants). The instant invention unexpectedly overcomes the decrease in yield that normally occurs in plants that accumulate large amounts of starch in their leaves. For example, currently plants exist that accumulate high amounts of starch in their leaves because they are unable to degrade it. Because of the inability of these plants to degrade their starch throughout their life they have poor growth and yield. The solution to the problem is provided herein and results in accumulation of starch only later in the life (e.g., after grain production) of the altered plant and the elimination/reduction of a decrease in growth and/or yield normally observed in high starch accumulating plants. Accumulation of starch occurs through genetic knock-down of one or more genes involved in normal turnover of starch through, for example, an RNAi approach.

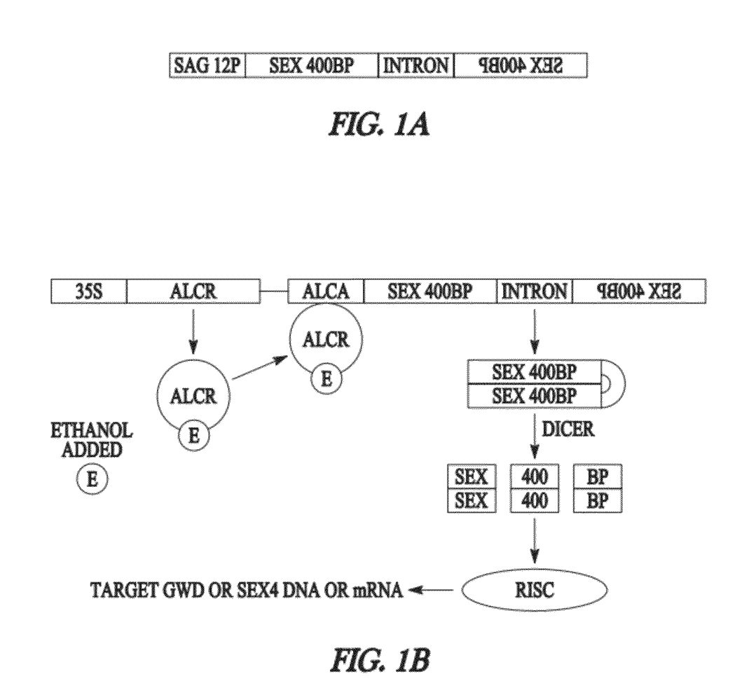

In general, plants that have high levels of starch in the leaves are generated as described herein by inhibiting starch degradation through the blocking of expression of glucan water dikinase, phosphoglucan phosphatase, β-amylase and/or other proteins involved in starch degradation. For example, the expression of the genes can be blocked by RNAi or antisense technology. Timing of the expression of the trait can be accomplished by several alternate mechanisms. For example, the gene expression blocking constructs can be coupled to an inducible promoter, for example the senescence induced promoter of the gene SAG12 or an alcohol inducible promoter. The promoter will drive expression of the starch degradation gene expression blocking constructs late in the developmental life cycle of the plant. The timing will ensure that the plant has had adequate time to turnover starch in order to promote growth, but will block starch degradation late in the life cycle of the plant so that the final harvested leaf material that will be used to produce ethanol or feedstock will contain an excess (increased) quantity of starch. For exemplary purposes only, an embodiment of the invention is demonstrated via RNAi against the glucan water dikinase enzyme (GWD) and the phosphoglucan phosphatase enzyme. Both enzymes play a role in starch degradation in plant leaves. These RNAi constructs were coupled to a senescence induced promoter (SAG12) or an alcohol-inducible promoter (FIG. 1).

An alternative embodiment provides for a plant in which a gene involved in starch degradation is knocked-out, suppressed/inhibited, or made non-functional by other means. This plant is then “rescued” by a transgene coding for the non-functional gene. This rescuing transgene can be under the control of a promoter that is active during the development of the plant, but becomes down regulated prior to (such as just/immediately prior to) or at senescence.

In describing the invention, the following terms will be employed and are defined as indicated:

As used herein, “heterologous” in reference to a nucleic acid is a nucleic acid that originates from a foreign species, or, if from the same species, is modified from its native form in composition and/or genomic locus by deliberate intervention. For example, a promoter operably linked to a heterologous structural gene is from a species different from that from which the structural gene was derived or, if from the same species, one or both are modified from their original form. A heterologous protein may originate from a foreign species or, if from the same species, is modified from its original form by deliberate intervention.

RNAi is a mechanism that inhibits gene expression at translation or by hindering transcription. Double-stranded RNA is synthesized with a sequence complementary to a gene of interest and introduced into a cell or organism, where it is recognized as exogenous genetic material and activates the RNAi pathway. The RNAi pathway is found in many eukaryotes and is initiated by the enzyme dicer, which cleaves double-stranded RNA (dsRNA) molecules. One of the two strands of each fragment, known as the guide strand, is then incorporated into the RNA-induced silencing complex (RISC).

In one embodiment, double stranded RNA is generated by constructing an inverted repeat with about 400 bp of DNA complementary to a target gene in the forward orientation and about 400 bp of DNA (the construct can include fewer or more than 400 bp complementary to a target gene) in the reverse orientation separated by, for example, an intron. The double stranded RNA is cleaved into short double stranded RNA molecules by the enzyme dicer. Single strands of RNA are then incorporated in the RNA-induced silencing complex (RISC). The RISC targets the complimentary mRNA or DNA to inhibit/prevent translation and/or transcription. The active components of an RNA-induced silencing complex (RISC) are endonucleases called argonaut proteins, which cleave the target mRNA strand complementary to their bound siRNA.

Any method to reduce expression/activity of the target gene is contemplated in the methods described herein. For example, in addition to RNAi, and rescue of a knock-out or knock-down gene, it also contemplated that sense suppression/cosuppression, antisense suppression, double-stranded RNA Interference, hairpin RNA Interference and intron-containing hairpin RNA Interference, Amplicon-Mediated Interference, and/or Small Interfering RNA or Micro RNA technology can also be used in the methods described herein.

By “host cell” is meant a cell which contains a vector and supports the replication and/or expression of the vector. Host cells may be prokaryotic cells such as E. coli, or eukaryotic cells such as yeast, insect, plant, amphibian, or mammalian cells. Preferably, host cells are monocotyledonous or dicotyledonous plant cells, including but not limited to maize, sorghum, sunflower, soybean, wheat, alfalfa, rice, cotton, canola, barley, millet, and tomato. A particularly preferred monocotyledonous host cell is a maize host cell.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed.

The terms “isolated” refers to material, such as a nucleic acid or a protein, which is substantially or essentially free from components which normally accompany or interact with it as found in its naturally occurring environment. The isolated material optionally comprises material not found with the material in its natural environment.

As used herein, “nucleic acid” includes reference to a deoxyribonucleotide or ribonucleotide polymer in either single- or double-stranded form, and unless otherwise stated, encompasses known analogues having the essential nature of natural nucleotides in that they hybridize to single-stranded nucleic acids in a manner similar to naturally occurring nucleotides (e.g., peptide nucleic acids). Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are “polynucleotides” as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass known analogs of natural amino acids that can function in a similar manner as naturally occurring amino acids.

The terms “polypeptide,” “peptide,” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “operably linked” includes reference to a functional linkage between a first sequence, such as a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

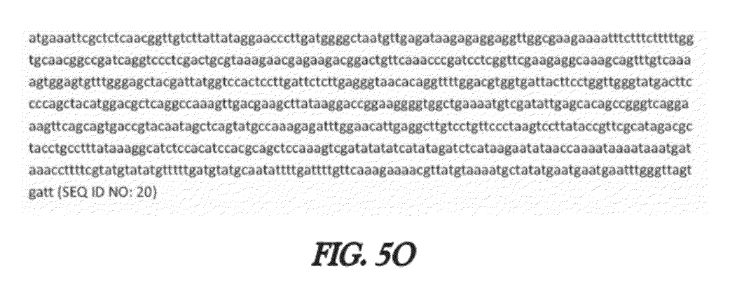

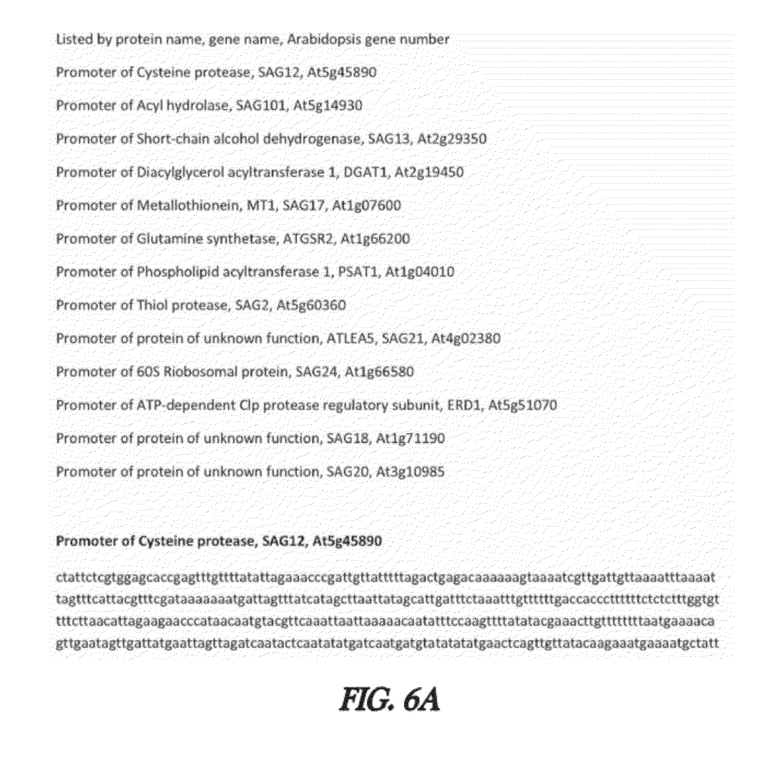

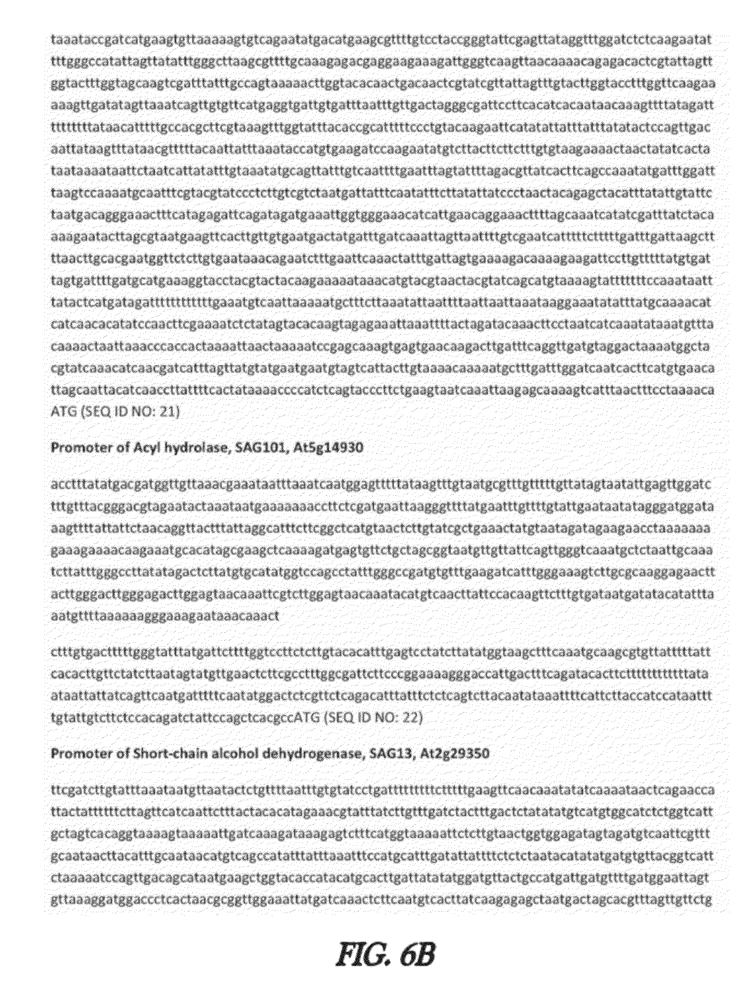

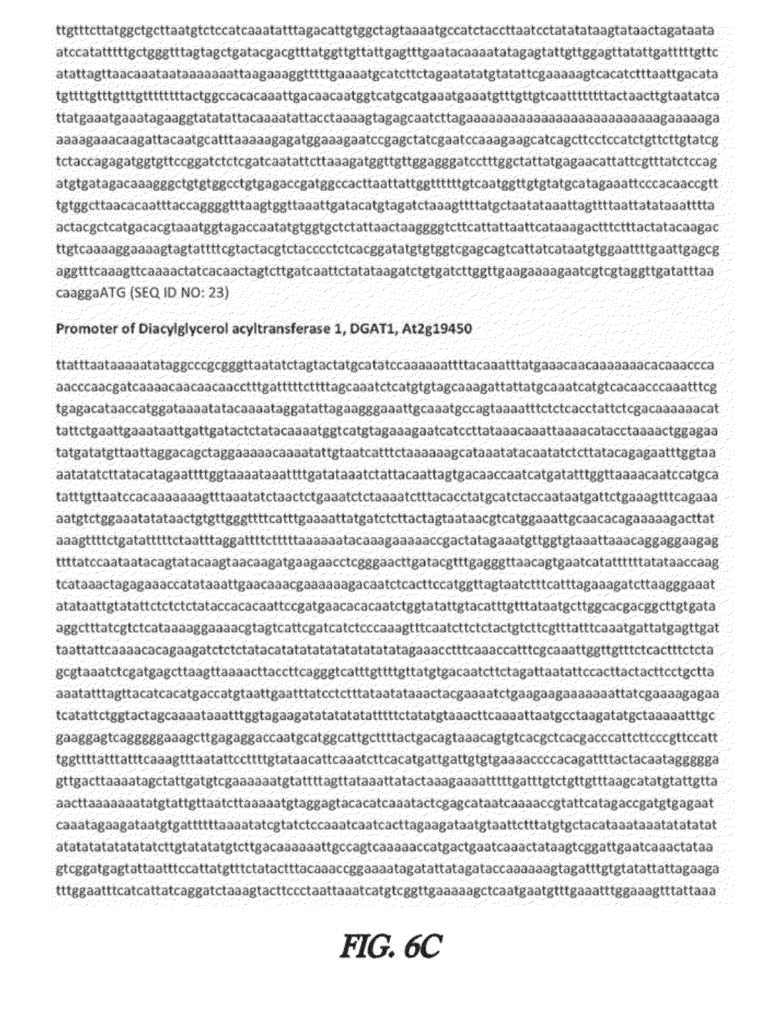

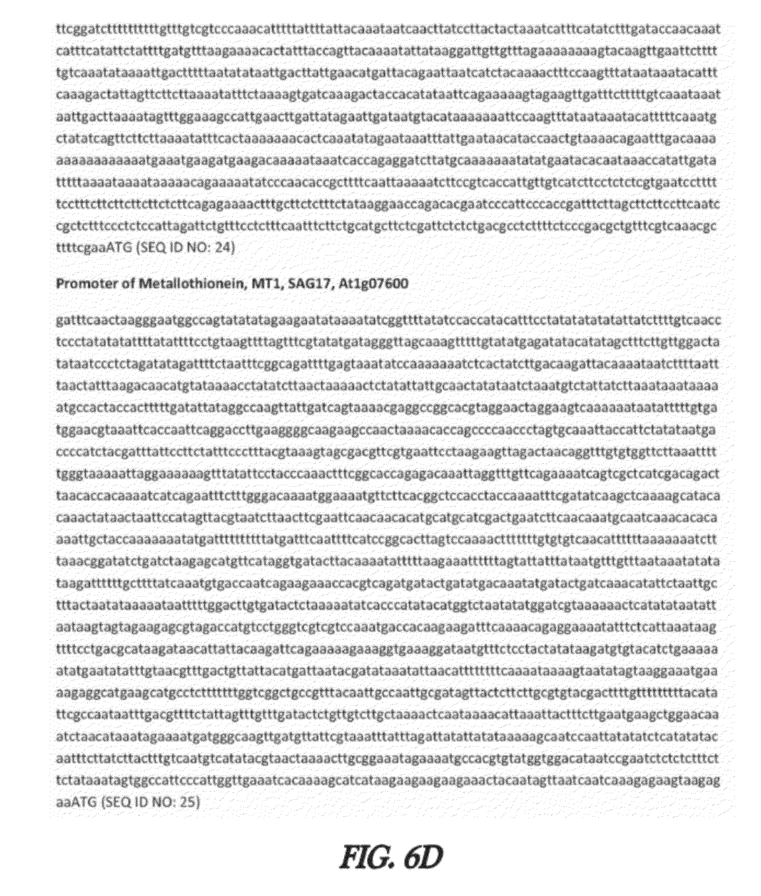

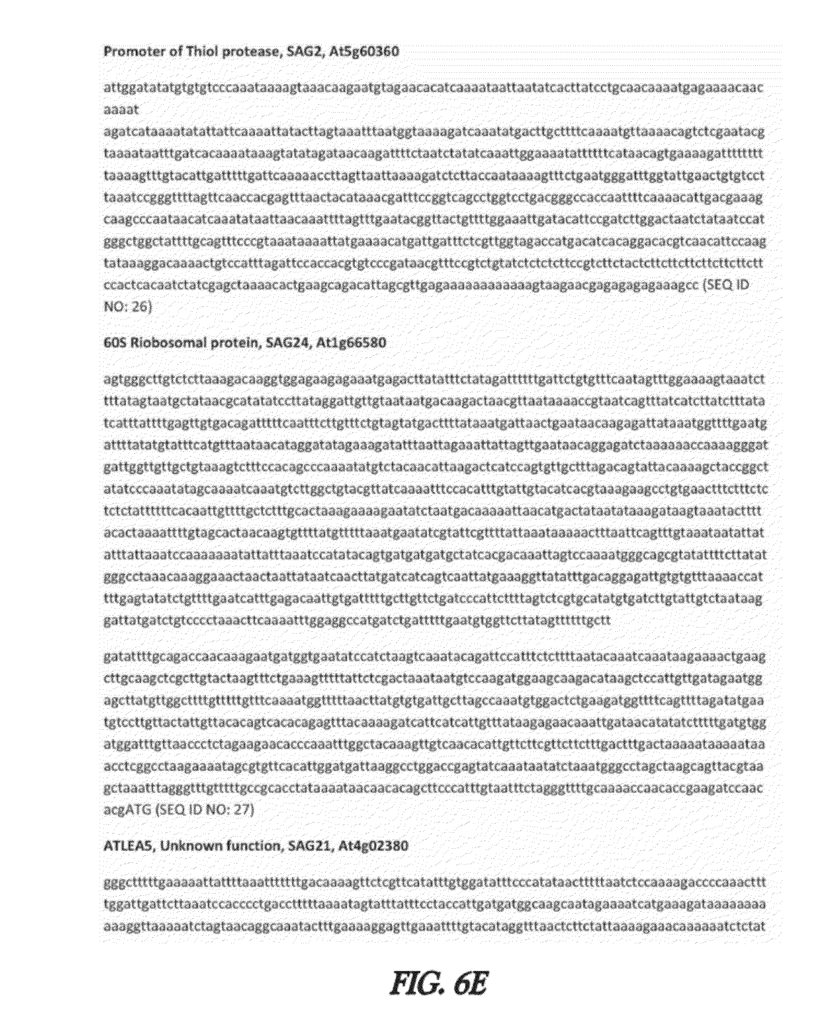

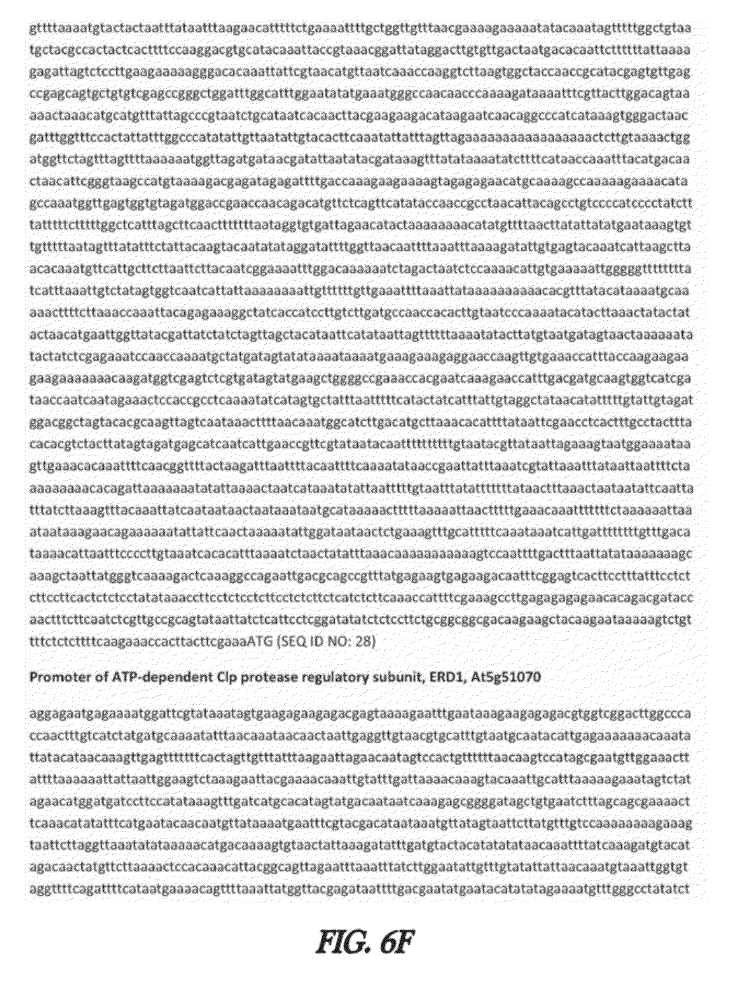

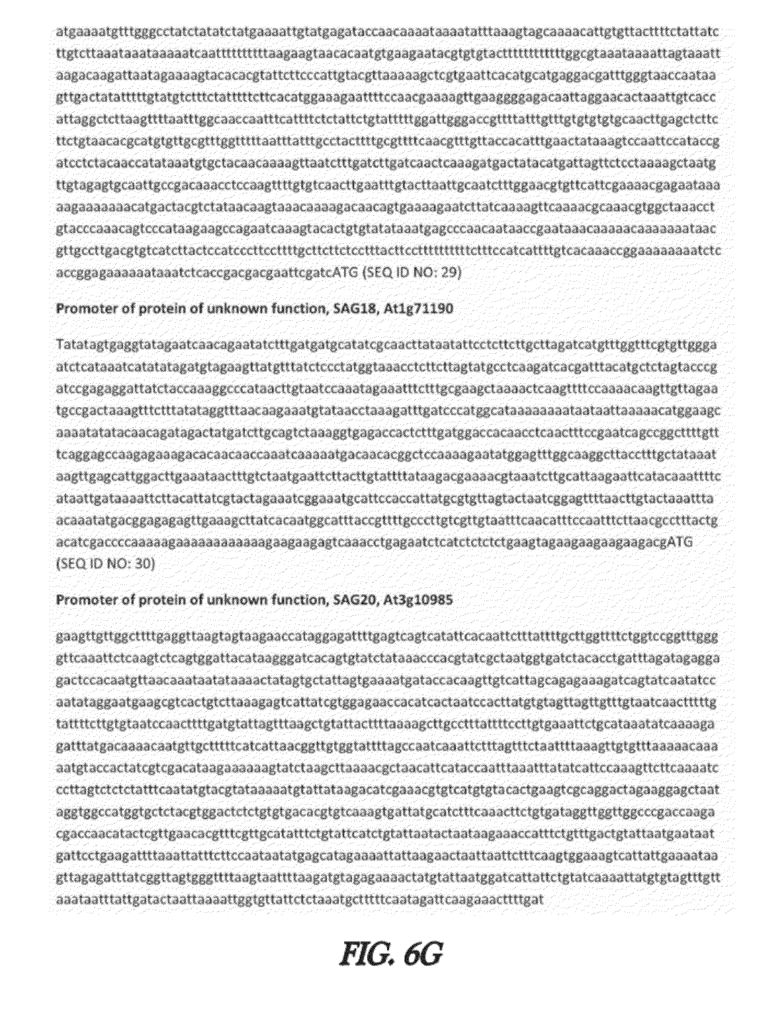

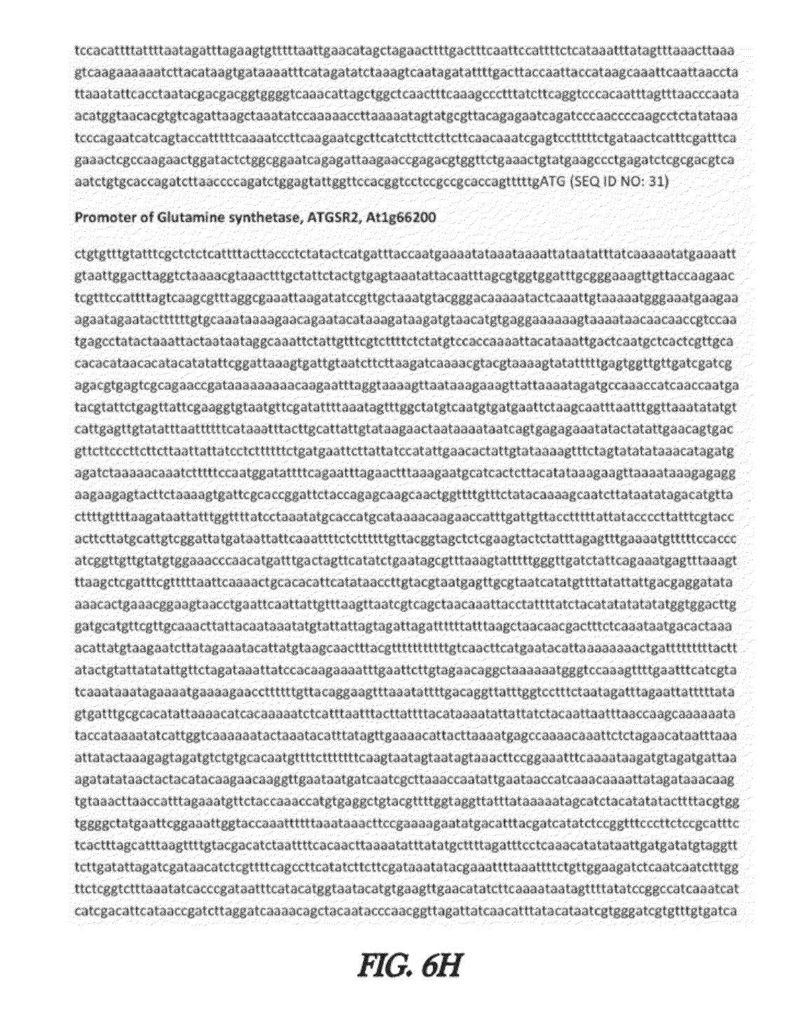

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to those that are obtained from plants, plant viruses, and bacteria which comprise genes expressed in plant cells such Agrobacterium or Rhizobium. Examples are promoters that preferentially initiate transcription in certain tissues, such as leaves, roots, seeds, fibers, xylem vessels, tracheids, or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental/external signal control (e.g., chemically inducible promoter, such as alcohol inducible promoter). Examples of environmental conditions that may effect transcription by inducible promoters include anaerobic conditions or the presence of light. Additional examples of inducible promoters are the Adh1 promoter, which is inducible by hypoxia or cold stress, the Hsp70 promoter, which is inducible by heat stress, the PPDK promoter, which is inducible by light, and the promoter inducible by alcohol. Another type of promoter is a developmentally regulated promoter, for example, a promoter that drives expression during pollen development or senescence (see, for example, FIG. 6). Tissue preferred, cell type specific, developmentally regulated, and inducible promoters constitute “non-constitutive” promoters. A “constitutive” promoter is a promoter, which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate intervention.

As used herein, “vector” or “plasmid” includes reference to a nucleic acid used in transfection of a host cell and into which can be inserted a polynucleotide. Vectors are often replicons. Expression vectors permit transcription of a nucleic acid inserted therein. Typical vectors useful for expression of genes in higher plants are well known in the art and include vectors derived from the tumor-inducing (Ti) plasmid of Agrobacterium tumefaciens described by Rogers, et al., (1987) Meth. Enzymol. 153:253-77. These vectors are plant integrating vectors in that on transformation, the vectors integrate a portion of vector DNA into the genome of the host plant. Exemplary A. tumefaciens vectors are plasmids pKYLX6 and pKYLX7 of Schardl, et al., (1987) Gene 61:1-11, and Berger, et al., (1989) Proc. Natl. Acad. Sci. USA, 86:8402-6.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter. For example, plant expression vectors may include (1) a pre-selected sequence under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

Expression cassettes, vectors and in vitro culture methods for plant cell or tissue transformation and regeneration of plants are known and available to the art. See, e.g., Gruber, et al., “Vectors for Plant Transformation,” in METHODS IN PLANT MOLECULAR BIOLOGY AND BIOTECHNOLOGY, supra, pp. 89-119.

As used herein, the term “plant” includes reference to whole plants, plant organs (e.g., leaves, stems, roots, etc.), seeds and plant cells and progeny of same. Plant cell, as used herein includes, without limitation, seeds, cultures, embryos, meristematic regions, callus tissue, leaves, roots, shoots, gametophytes, sporophytes, pollen, and/or microspores. The class of plants, which can be used in the methods of the invention, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium, and Triticum. In one embodiment, the plant is Zea mays. In another embodiment, the plant is Arabidopsis.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic.

“Silage” is fermented fodder that can be fed to, for example, ruminants (cud-chewing animals like cattle and sheep) or used as a biofuel feedstock. It is fermented and stored in a process called ensilage or silaging, and usually made from grass crops, including corn (maize) or sorghum or other cereals, using the entire green plant (not just the grain). Silage can be made from many field crops, and special terms may be used depending on type (oatlage for oats or haylage for alfalfa).

“Bio-alcohols,” such as “bio-ethanol,” are biologically produced alcohols, most commonly ethanol, and less commonly propanol, butanol and methyl butenol, which are generally produced by the action of microorganisms and enzymes through the fermentation of sugars or starches, or cellulose. Ethanol fuel is the most common biofuel worldwide. Alcohol fuels are produced by fermentation of sugars derived from wheat, corn, sugar beets, sugar cane, molasses and any sugar or starch that alcoholic beverages can be made from (like potato and fruit waste, etc.). The ethanol production methods used are enzyme digestion (to release sugars from stored starches), fermentation of the sugars, distillation and drying. The distillation process requires energy input for heat (often unsustainable natural gas fossil fuel, but cellulosic biomass, such as the waste left after sugar cane is pressed to extract its juice, can also be used more sustainably). Ethanol can be used in engines as a replacement for gasoline.

“Biomass” or “biofuel” is material derived from recently living organisms. This includes plants, animals and their by-products. For example, manure, garden waste and crop residues are all sources of biomass. It is a renewable energy source based on the carbon cycle, unlike other natural resources such as petroleum, coal, and nuclear fuels. It is used to produce power, heat and steam and fuel, through a number of different processes. “Agrofuels” are “biofuels” which are produced from crops. There are two common strategies of producing liquid and gaseous agrofuels. One is to grow crops high in sugar (sugar cane, sugar beet, and sweet sorghum) or starch (corn/maize), and then use yeast fermentation to produce alcohol (e.g., ethanol). The second is to grow plants that contain high amounts of vegetable oil, such as oil palm, soybean, algae, jatropha, or pongamia pinnata. When these oils are heated, their viscosity is reduced, and they can be burned directly in a diesel engine, or they can be chemically processed to produce fuels such as biodiesel. Wood and its byproducts can also be converted into biofuels such as woodgas, methanol or ethanol fuel. It is also possible to make cellulosic ethanol from non-edible plant parts.

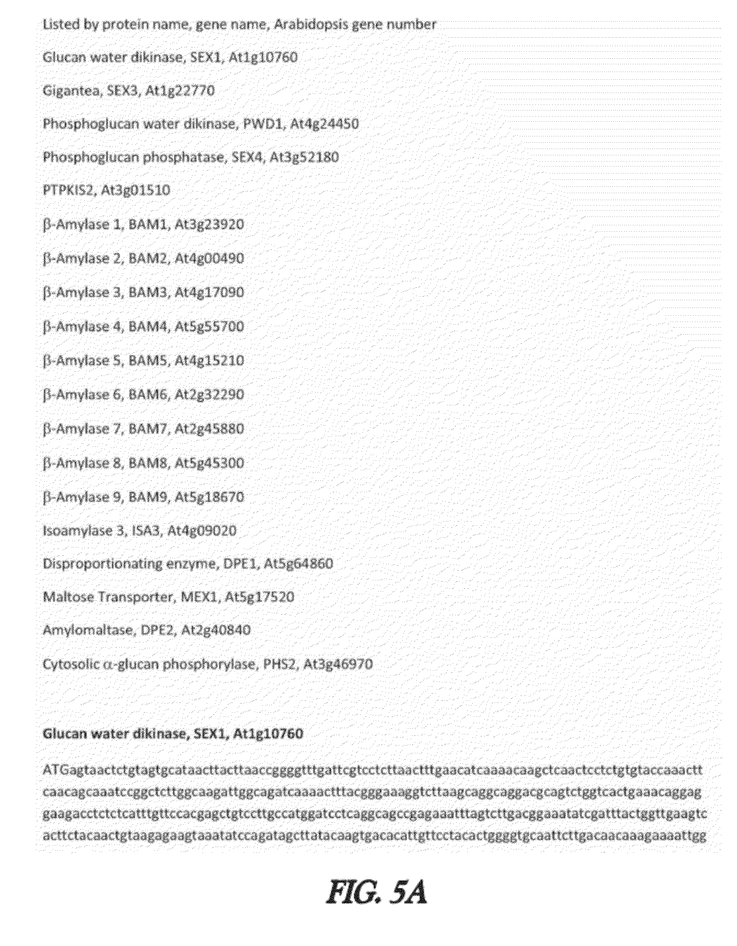

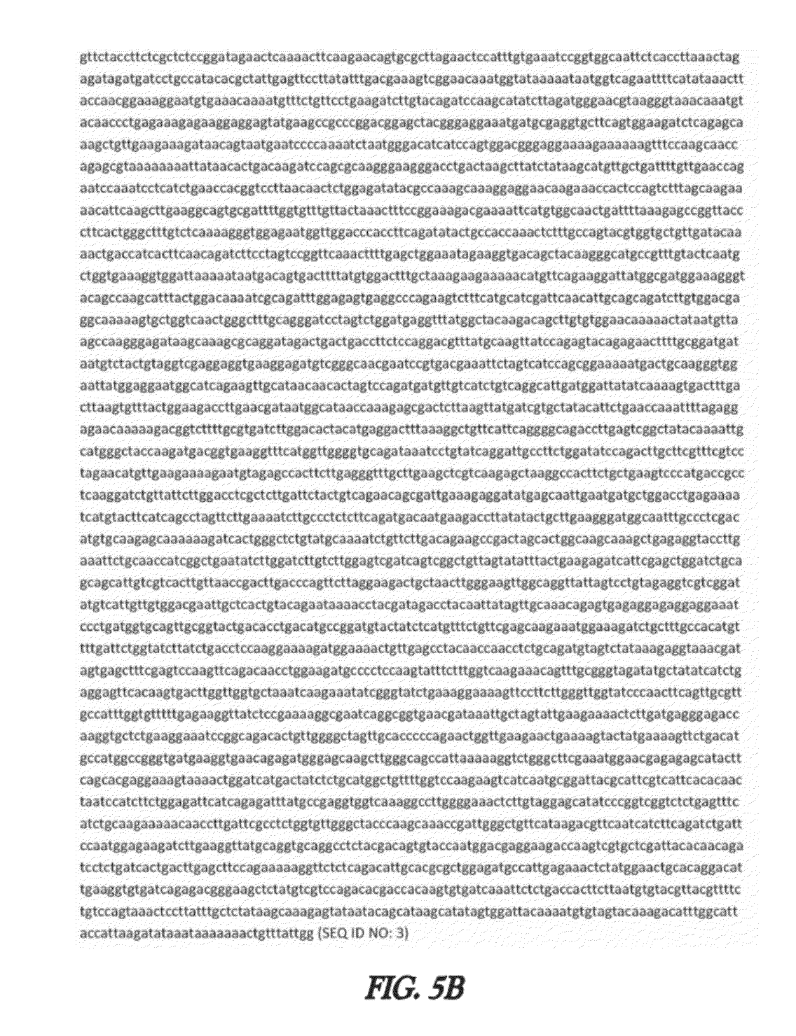

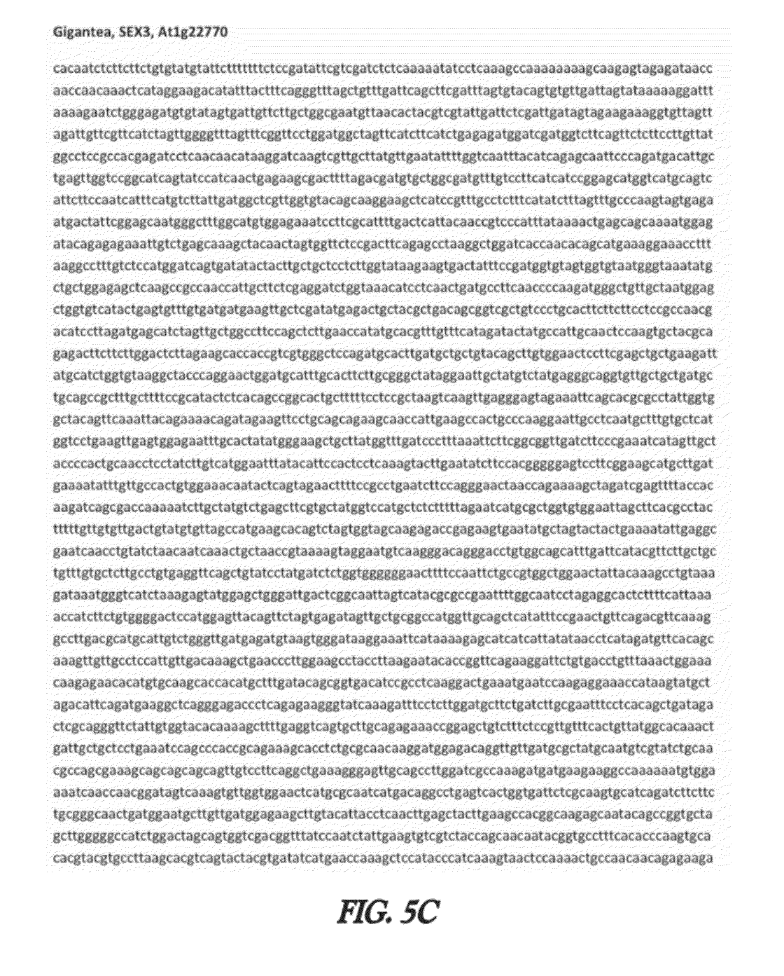

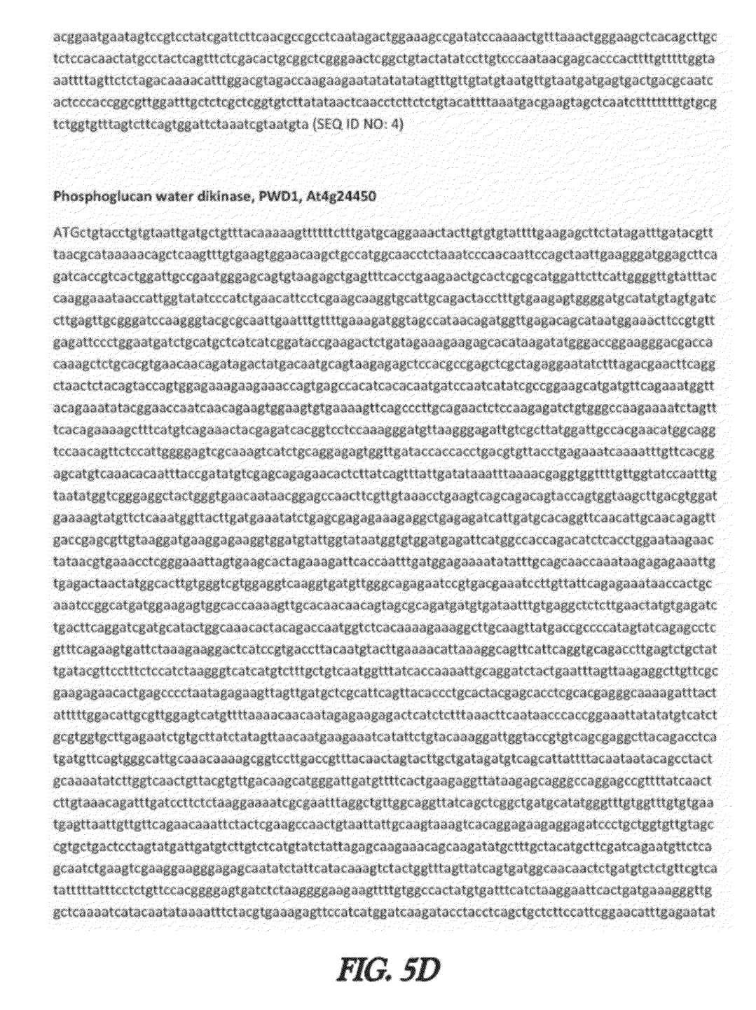

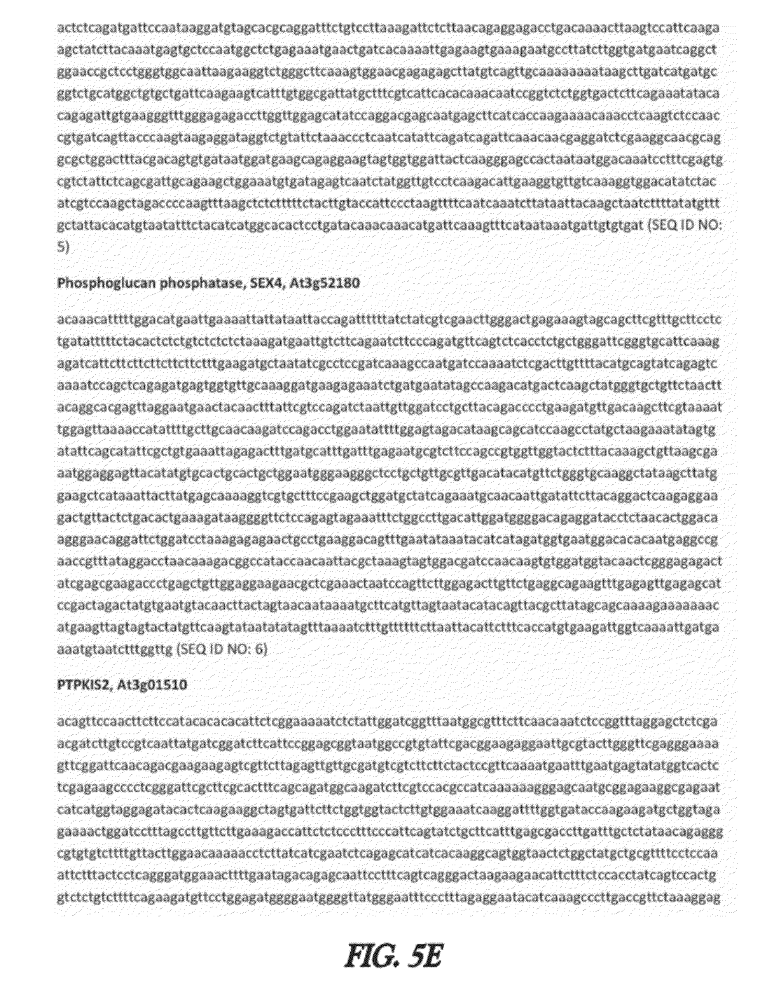

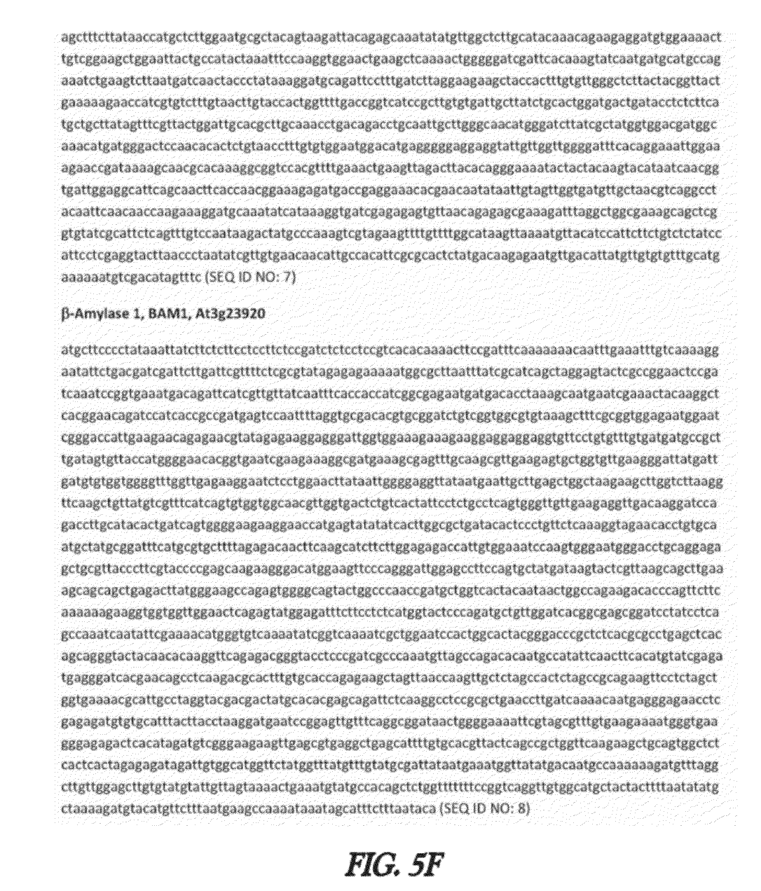

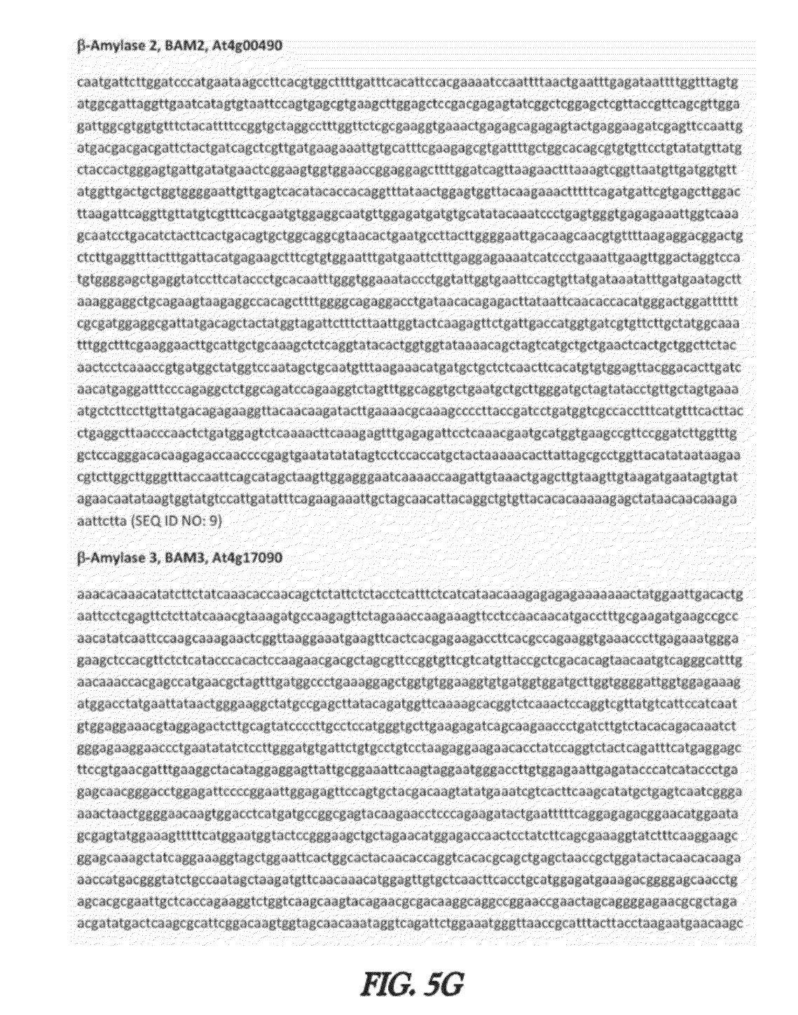

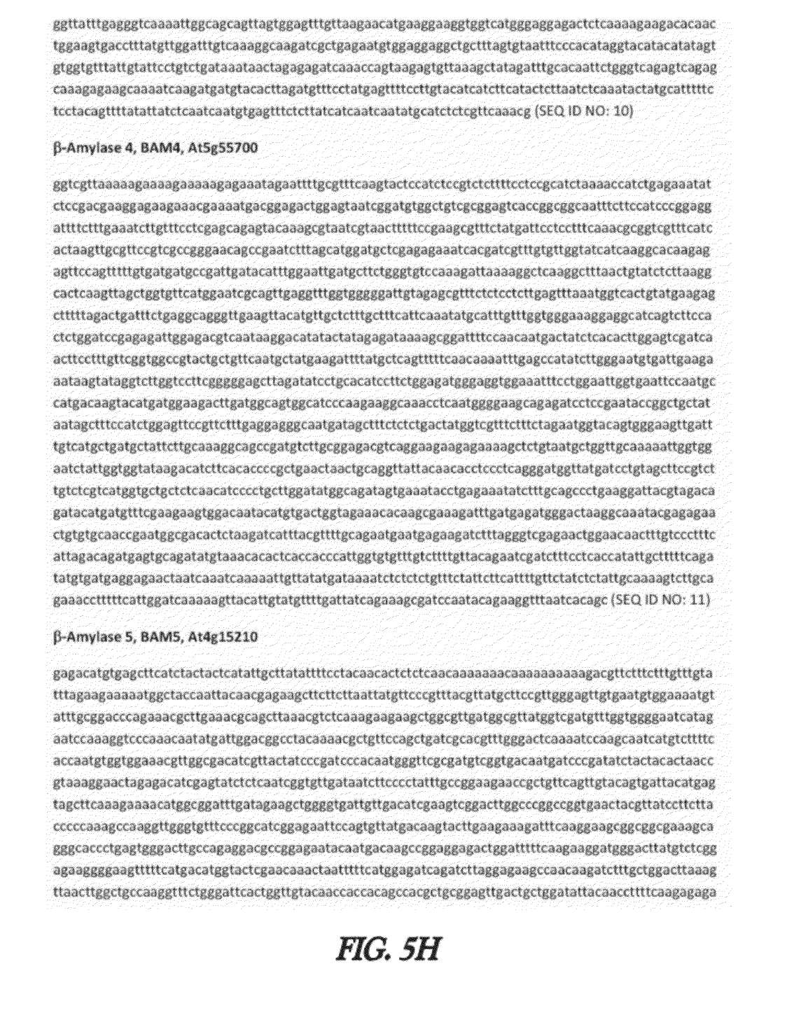

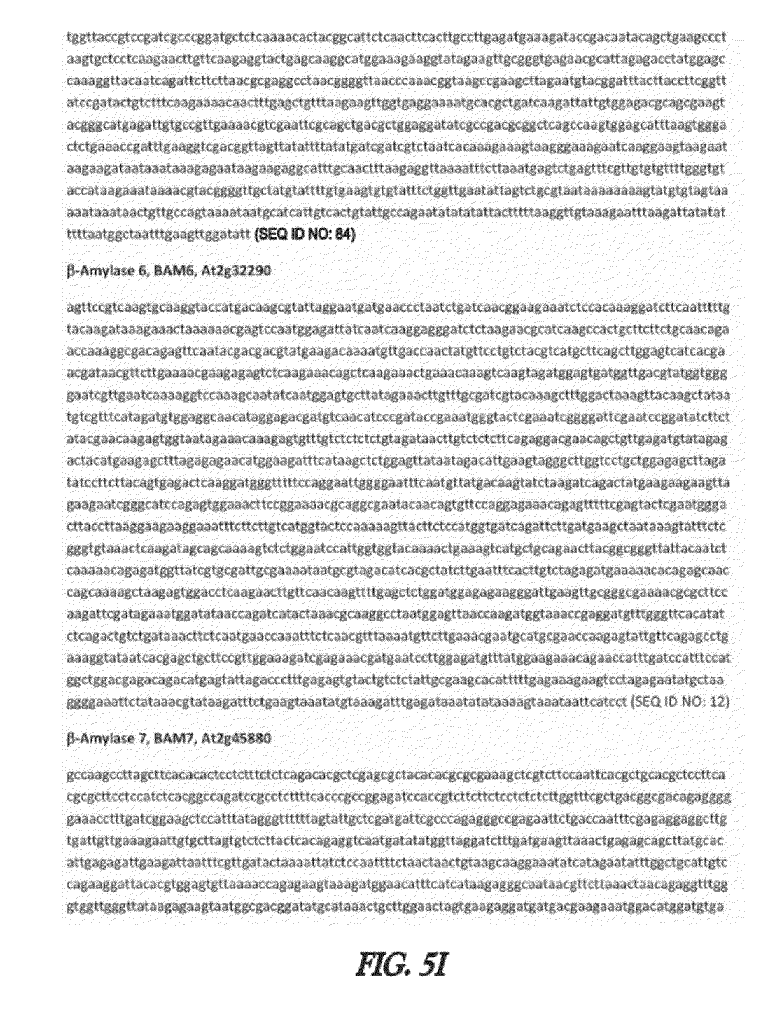

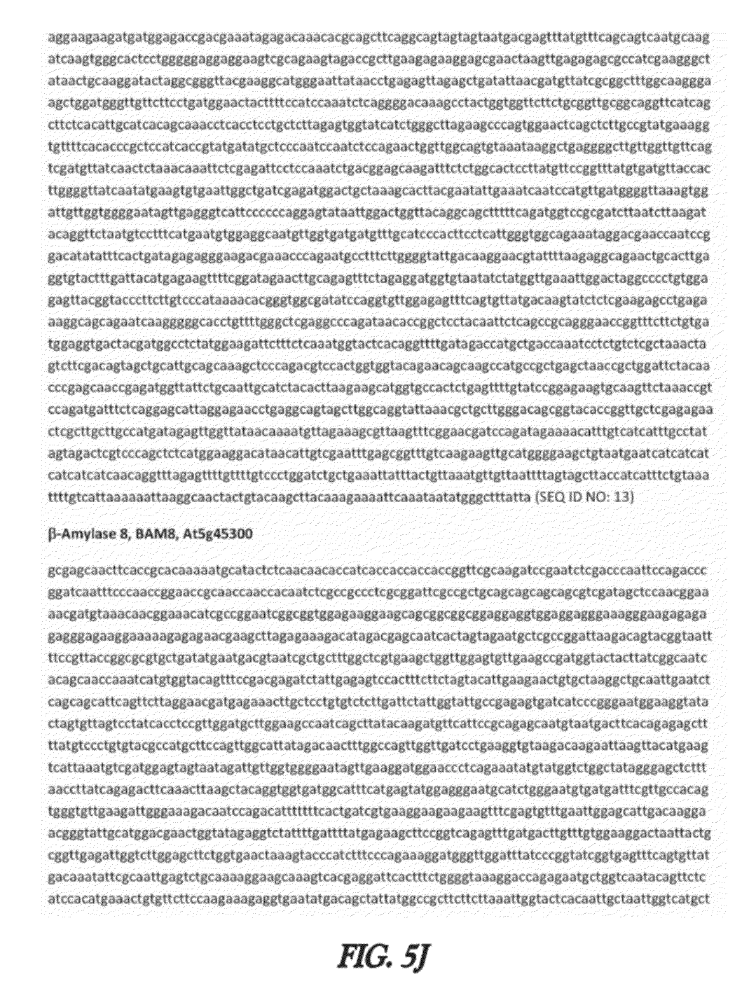

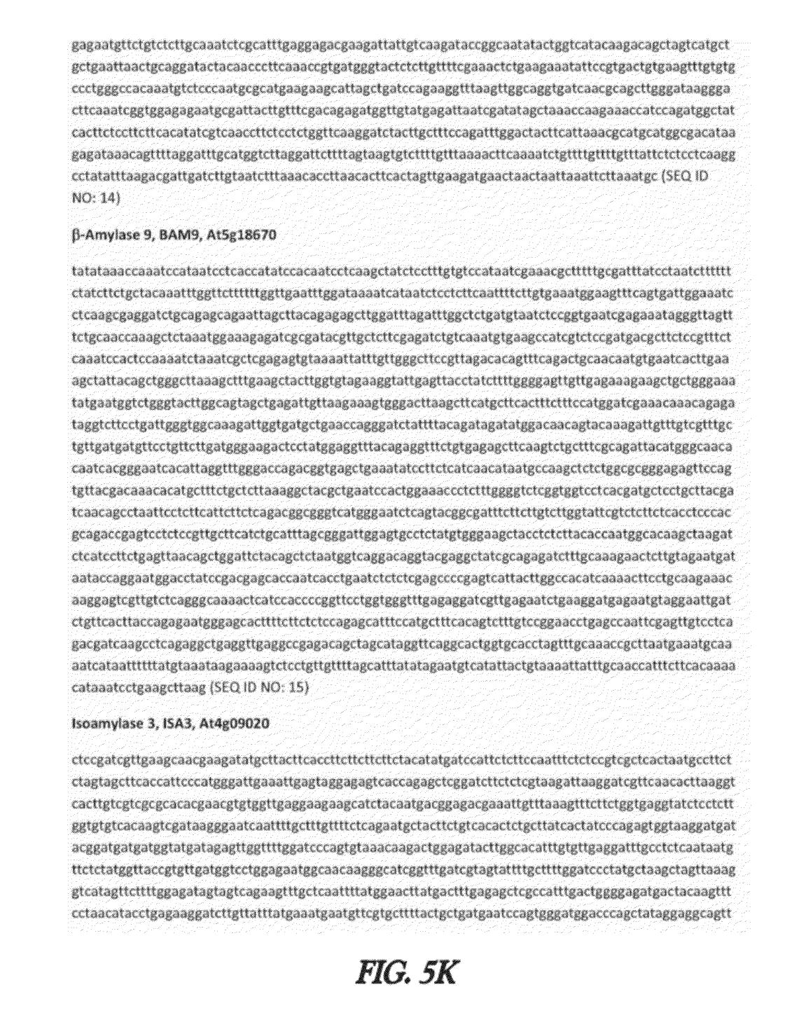

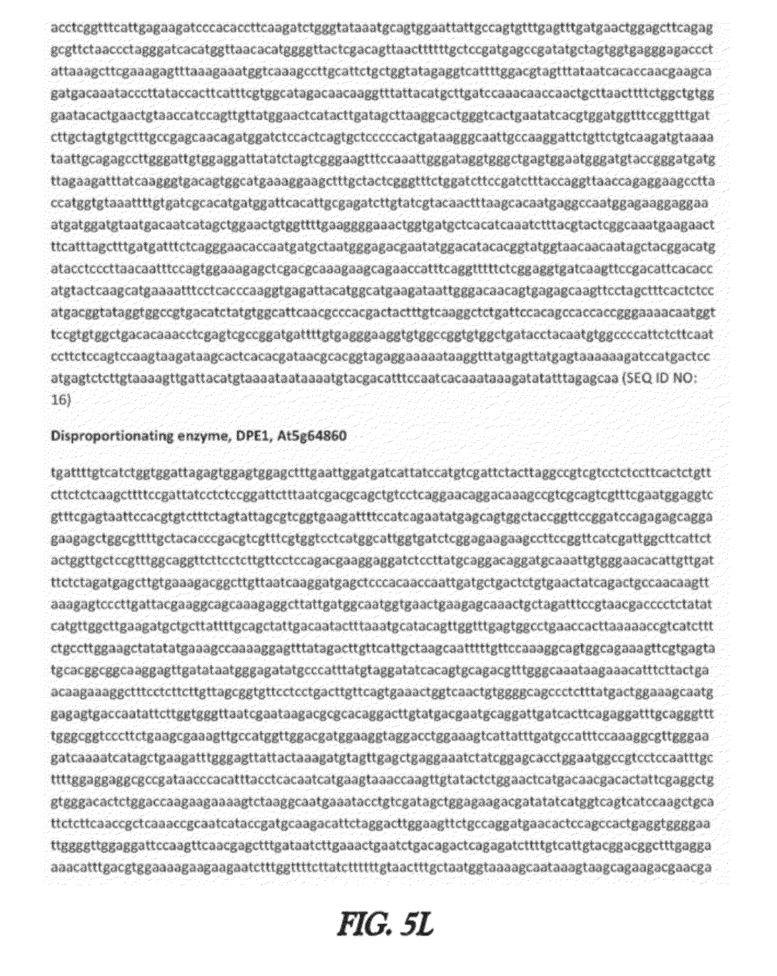

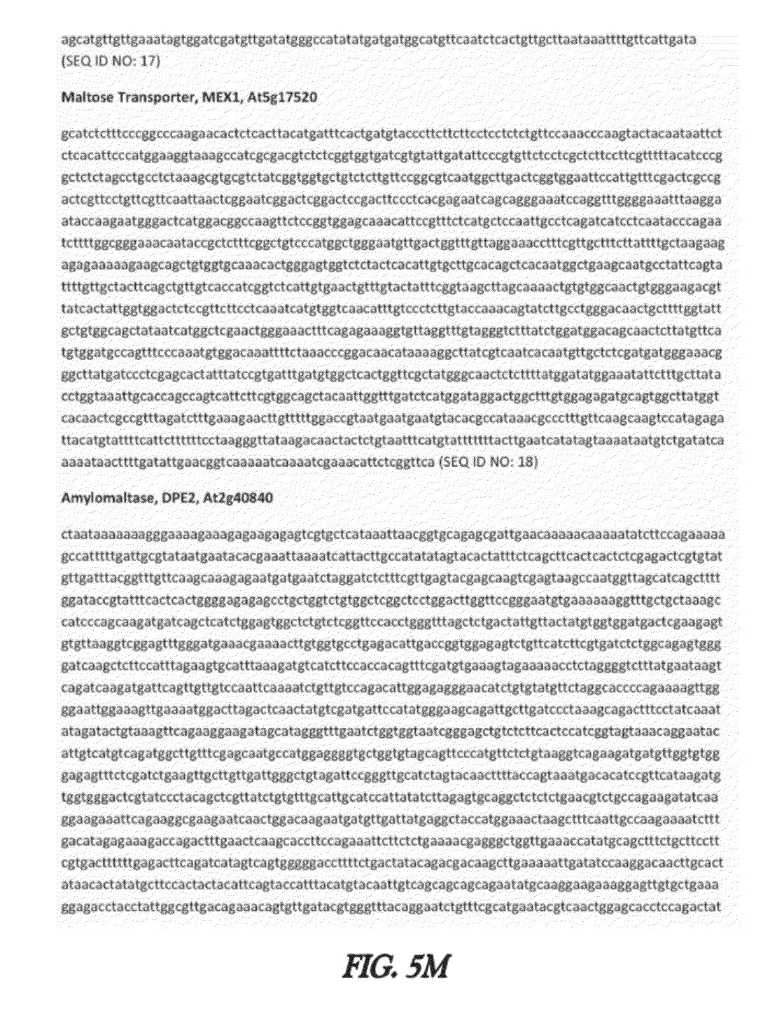

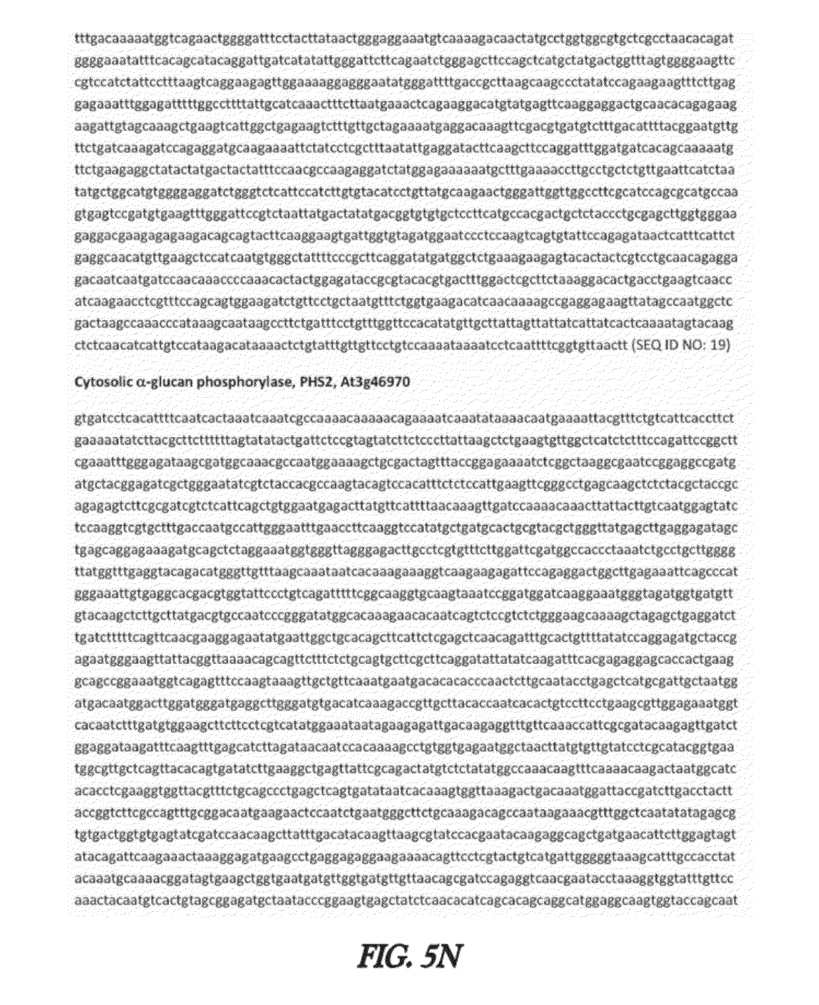

As discussed above, the target sequences can be glucan water dikinase, phosphoglucan phosphatase, β-amylase and/or other proteins involved in starch degradation. Examples of target sequences are provided in FIG. 5. The nucleic acids of the present invention can be made using (a) standard recombinant methods, (b) synthetic techniques, or combinations thereof that are available to the art.

Numerous methods for introducing foreign genes into plants (e.g., plant cells) are available to an art worker, including biological and physical plant transformation protocols. See, e.g., Miki, et al., “Procedure for Introducing Foreign DNA into Plants,” in METHODS IN PLANT MOLECULAR BIOLOGY AND BIOTECHNOLOGY, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium mediated transformation (Horsch, et al., (1985) Science 227:1229-31), microprojectile-mediated transformation, electroporation (Riggs, et al., (1986) Proc. Natl. Acad. Sci. USA 83:5602-5606), micro-injection (Crossway, et al., (1986) Biotechniques 4:320-334; and U.S. Pat. No. 6,300,543), direct gene transfer (Paszkowski, et al., (1984) EMBO J. 3:2717-2722), sonication (Zang, et al., (1991) BioTechnology 9:996), liposome or spheroplast fusions (Deshayes, et al., (1985) EMBO J. 4:2731; and Christou, et al., (1987) Proc. Natl. Acad. Sci. USA 84:3962), protoplast transformation, macroinjection, DNA uptake by germinating pollen and DNA uptake in embryos by swelling (Potrykus, Physiol. Plant (1990), 269-273), and biolistic bombardment (see, for example, Sanford, et al., U.S. Pat. No. 4,945,050; WO 91/10725; and McCabe, et al., (1988) Biotechnology 6:923-926; Tomes, et al., Direct DNA Transfer into Intact Plant Cells Via Microprojectile Bombardment. pp. 197-213 in Plant Cell, Tissue and Organ Culture, Fundamental Methods eds. O. L. Gamborg & G. C. Phillips, Springer-Verlag Berlin Heidelberg New York, 1995).

Once the DNA introduced is integrated into the genome of the plant cell, it is generally considered stable and is also retained in the progeny of the originally transformed cell. It can contain a selection marker which mediates, for example, resistance to a biocide such as phosphinothricin or an antibiotic such as kanamycin, G 418, bleomycin or hygromycin, to the transformed plant cells or which permits selection via the presence or absence of certain sugars or amino acids. The marker chosen should therefore allow the selection of transformed cells over cells which lack the DNA introduced.

Once transformed, these cells can be used to regenerate transgenic plants in any manner available to the art. Seeds may be obtained from the plant cells. Two or more generations can be grown in order to ensure that the phenotype characteristic is stably retained and inherited. Also, seeds can be harvested in order to ensure that the phenotype in question or other characteristics have been retained.

Transformed, transgenic and knockout plants produced according to the present invention include both monocot and dicot plants. Dicot plants include, but are not limited to, tobacco, potato, cabbage, soybeans, or sweet potato. Monocot plants include, but are note limited to, maize, wheat, barley, rice, oats or other small cereals. In one embodiment, the maize is Zea mays. In another embodiment, the plant is Arabidopsis. The invention also relates to propagation material of the plants according to the invention, for example fruits, seeds, tubers, rootstocks, seedlings, cuttings, calli, protoplasts, cell cultures, tissues and the like.

Numerous processes for extracting the starch from plants or starch-storing parts of plants are available to the skilled worker.

The plants produced by the methods herein provide a better and higher yielding feedstock for bio-ethanol production. For example, a dedicated crop or crop residue such as corn stover (leaves and stems) which has high starch content would provide a higher yield of fermentable sugars for the production of bio-ethanol due to an increase in sugar content and because starch is more easily hydrolyzed and used for fermentation than the cellulose of cell walls. The plants produced by the methods herein also provide a better animal feed. For example, specific maize varieties for use as silage or forage with high starch content would reduce feed cost for dairy production by providing an alternative rapidly digestible carbohydrate source, allowing the reduction or elimination of grain supplements.

As used herein, “a decrease” in yield or growth refers to the amount of yield (e.g., grain/crop yield) or growth loss that can be accommodated due to an increase in starch yield. For example, in one embodiment, “a decrease” in yield or growth is about 1% to about 10%, or less than about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% or higher such as less than about 35%, about 40% about 45%, or about 50% loss in yield (e.g., grain/crop yield) or growth, as compared to a wild-type plant.

“Inhibit expression” refers to a decrease in expression of protein or RNA. This inhibition can be from about 0.5% to 100% inhibition of expression of the gene, including about 1% to about 10%, or about 20%, about 25%, about 30%, about 35%, about 40%, about 45%, about 50%, about 55%, about 60%, about 65%, about 70%, about 75%, about 80%, about 85%, about 90%, about 95%, or about 100%.

In one embodiment, the sequences mentioned herein also comprise variations that are at least about 50% or about 60% or about 70%, about 71%, about 72%, about 73%, about 74%, about 75%, about 76%, about 77%, about 78%, or about 79%, or at least about 80%, about 81%, about 82%, about 83%, about 84%, about 85%, about 86%, about 87%, about 88%, or about 89%, or at least about 90%, about 91%, about 92%, about 93%, or about 94%, or at least about 95%, about 96%, about 97%, about 98%, or about 99% sequence identity, compared to the sequences provided herein using one of alignment programs available in the art using standard parameters. In one embodiment, the differences in sequence are due to conservative amino acid changes. In another embodiment, the protein sequence has at least 80%, or at least 85%, at least 90% or at least 95% sequence identity with the sequences provided herein and can be bioactive.

Methods of alignment of sequences for comparison are available in the art. Thus, the determination of percent identity between any two sequences can be accomplished using a mathematical algorithm. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

### Examples

The following examples are provided in order to demonstrate and further illustrate certain embodiments and aspects of the present invention and are not to be construed as limiting the scope thereof.

### Introduction

Plants make many sugars and polysaccharides that are easily fermented to ethanol, but in most cases only cellulose is left in the parts of the plant not used for food at harvest. Cellulose is the largest store of carbohydrate in non-food plant parts, and therefore presents is a target for biofuel conversion. However, any additional carbon that can be stored and retained in the leaf as a more easily digestible polysaccharide, such as starch, has the potential to increase the yield of biofuel. The problem with leaf starch accumulation is a yield penalty. Disclosed herein are methods based on genetic engineering of RNAi that overcomes the yield penalty associated with starch accumulation in leaves. In addition, the possibility of using biomass for co-firing in conventional power plants has gained increasing traction in recent years and interest in this area will only continue to grow as battery technology improves and net carbon dioxide emissions are capped (Ohlrogge et al. 2009). For biofuel production or for biomass burning increasing the energy density of above ground biomass is a highly desirable goal.

Plants that accumulate starch in their leaves are well known. Many mutations lead to a starch excess phenotype and starch can make up 50% of the dry weight of leaves (Messerli et al. 2007). The major disadvantage of these plants is that they have a significant yield penalty, such that total fuel yield is reduced. Some of the highest starch accumulations are found in plants in which transitory (leaf) starch phosphate metabolism has been disrupted. Leaf starch differs from grain starch in having a small amount of phosphate. This phosphate is added by a glucan-water dikinase (GWD). The GWD enzyme is coded for by the gene that is lost in the Arabidopsis mutant called Starch Excess 1 (SEX1) (Ritte et al. 2002). A phosphoglucan phosphatase coded for by the gene SEX4 is also needed for the breakdown of leaf starch. The phosphoglucan phosphatase removes the phosphate groups added to the starch by the GWD (Kotting et al. 2009). When GWD or the phosphoglucan phosphatase is absent, plants accumulate starch in their leaves.

The basic premise is the significant yield penalty observed in starch accumulating mutants can occur because starch accumulates early in the plant's life cycle and this prevents effective investment in productive leaf biomass. A disclosed herein, the inventors have discovered that yield penalty can be reduced or eliminated by delaying starch accumulation until after the plant has reached a certain size, making leaf starch accumulation a desirable trait in a biofuels crop. The approach is to use inducible RNA interference (RNAi) constructs against the GWD and phosphoglucan phosphatase enzymes. RNAi is a mechanism that inhibits gene expression at translation or by hindering transcription. In the RNAi constructs, double stranded RNA is generated by making an inverted repeat with ≈400 bp of DNA identical to target gene in the forward orientation and 400 bp of DNA in the reverse (complement) orientation separated by an intron. These inverted repeats are placed behind an inducible promoter (FIG. 1). When the promoter is induced the inverted repeat DNA is transcribed and the resulting RNA molecule snaps back onto itself forming double stranded RNA. The double stranded RNA is cleaved into short, about 20 bp, double stranded RNA molecules by the enzyme dicer. Single stands of these short RNA fragments are then incorporated in the RNA-induced silencing complex (RISC). The RISC targets the complimentary mRNA or DNA to prevent transcription and/or translation.

A promoter that is activated during senescence and a promoter that is activated by the addition of exogenous ethanol were used. The senescence-inducible promoter is active late in the life cycle of the plant near the time of senescence (Noh & Amasino 1999). During the early stages of growth, starch will increase and decrease each day following the normal pattern and allowing for maximal leaf growth. However, once the plant begins to senesce, this promoter will be induced (Gan & Amasino 1995) causing expression of the inverted repeat, leading to reduced starch degradation. Using an auto-inducible promoter such as the senescence-inducible promoter outlined above is a desirable feature in a starch or any other higher energy density biomass accumulating biofuel crop as no costly or time consuming intervention by the farmer would be necessary.

Both Arabidopsis and maize plants were engineered to accumulate leaf starch. The timing of starch accumulation with an alcohol-inducible promoter was also altered. To test starch accumulation in a crop plant we made an RNAi construct against a homologous gene in maize to the Arabidopsis SEX1 gene. It was determined that the alcohol inducible promoter does not work in maize and senescence inducible promoters are not yet known in maize. Therefore, the RNAi construct was placed behind the constitutive ubiquitin promoter. It is demonstrated herein that plants can be engineered to accumulate starch in the leaves with little impact on total biomass yield.

### Materials and Methods

RNAi Design

Arabidopsis

The RNA interference (RNAi) construct which forms the DNA inverted repeat consists of a 400 bp sequence which is homologous to portions of the 5′ UTR and coding regions of the target gene. This is followed by a 779 bp long pyruvate dehydrogenase kinase intron (Pdk) followed by the inverse repeat of the first 400 bp.

The RNAi against the gene Starch Excess 1 (SEX1) in Arabidopsis (At1g10760) was designed using 85 bp of DNA sequence directly upsteam of the ATG start site followed by the first 230 bp of genomic SEX1 sequence followed by an 85 bp sequence which was taken starting 569 bp upstream of the ATG start site. The last 85 bp of the discontinuous sequence was chosen to avoid homology with, and possible silencing of, the PTPKIS2 gene (At3g01510). The RNAi against the gene Starch Excess 4 (SEX4) in Arabidopsis (At3g52180) was designed using 253 bp of DNA sequence directly upstream of the ATG start site followed by the first 165 bp downstream of the ATG start site, of genomic SEX4 sequence. The inverted repeats were placed behind either a senescence inducible promoter or an alcohol inducible promoter with an octopine synthase terminator (3′ OCS). (FIG. 1a)

The senescence-inducible promoter is the promoter of the senescence associated gene 12 (SAG12) in Arabidopsis At5g45890 (Noh and Amasino, 1999). The sequence of the SAG12 promoter was provided by Dr. Rick Amasino at the University of Wisconsin-Madison and can be found in GenBank under accession number U37336 (from 1 to 2186).

The alcohol-inducible promoter is from the fungus Aspergillus nidulans (Felenbok, 1991). The sequence of the Alc promoter system was found in the EMBL database. The Alc system consists of two parts (FIG. 1b). The first part is a gene AlcR which encodes for an ethanol binding protein and was placed behind a single 35S full length promoter. The second part is the AlcA promoter which responds to the AlcR protein when alcohol is present. The AlcA promoter was placed in front of our RNAi constructs.

AlcR (Codes for the Protein that when Bound to Alcohol Binds to the AlcA Promoter)

(SEQ ID NO: 34)
ctacaaaaagctgtcaactttcccattcaaacctctctctaaatcct
tcgcaaccagcgccgccatatccgacttcctgcccagaaattgcagc
gcccgaatgcagtagttgcaattttgccgcagctgcaggtaccccgc
tagtgcatttccttggccgtccagatctaaacagtccagcaagatat
acgcagctttggcaaacgagtgaataagaacgacggtccacggttct
accaggaatgccacctcggtcagagaatcatggaaatggcgatacat
cggagatgctttgcccgggtccagctcctggcctcggagtgaagagc
gcgcaagggcactaactgctagtgcattatcgagccttagctttgtt
acaaggtcgatgtggttgatatcagagtacgaatcgcggtcgatgct
ctccaaaacgtccgctagcaacatcgcggctagatgccagtgaccgt
ctagaatgacgtaccaagactggatgcgcgaagggaggagctcgtgg
ttagcaacgcagtcctgcataaatggttggtacttcgctgtccagtg
attataaacgtagagcgttctctggatggccgcttcaaggcgggcag
ggctggcgccgcgatagaggagggtttggagctgcgtgacgcggcgg
tagaggaggactttgacgggcgttcagaggagagagcggcggcggcc
tgttcgtaggtgcagggccatcgagccgctggctgagagatttgtgt
gtgggattcgtgatctgggagagagtccgaggtgcggaggaagaggt
cgccccatacgtcgctcttttcttgattgctcgggacctgtctcggg
ggctcccagcagtctaggttgatcggcgtttcagcgccgcgccttgg
tggagatgccgatgatatctggctatcctcatctgacaccacgagtg
gtcgctggtacattgcagcgcttagtgtatcgaacatgatccctagc
cagaacataaggcttaatgtactgcgatgctcctcactggccacaac
cgggtcaagctgtgggctttcagacgacggcgtcggtgtctcgaaaa
taccggcgaatgtcgatgcgacagatcctcccgggagcctgttgaaa
gccttaccgcggcgttgcattcgtgcaaacttatgtcggaatgtata
aagctgacggttcgcggtttccaggaacacgggcgcaccgtcatttt
cgagtagcttgtctagacgtgcacccataccgtgctgctcatcatca
tccagcacactctgcgtgagagaaaagatgatattcgcaaatataac
ccggaatgatggaatccctgtcgtgtgctgcaaggcatggcgtgctt
cattccaggcgttcctccggatggacctctcatcggcggctatgtct
gcaggaacatttagcccagcccccctctgcgcatgctgcgtccattg
cgacgcaaaagctacgatcgccagatgcagggctcgggctgcggctt
tgtcctcttccgcactcagggcgcgcccgcgtaatgaggtagatacg
cgatctagccggcacacccggatgcacatcctgtttgaccagttcgg
gccccattccgcccgctgcttgggcggcaggtagctgatctggtcgg
agtatggacaattgtgctctgtcagccagcaggacagtgcattctcc
atactatcgtggtagattcgcatcagattcttcgtcattgtggaacg
ggcatatcgccgtgctgtgctgtctgaggccatgcacagactgttct
caggcagtagcggcgactgccgatcagaccatagcgatccatcttga
gcgacctgttgtacactattagtatcggtaaccgagtccggatccgt
agtctgctcttccagctcgcgaatgaggtcatccgtgttcgggctat
ggggcggaagaaggactgcttgaaaactgagcggactgagaggtttc
tcgagctgttggcccatgctgaaatcaccggggatggctagatccca
cggaaaaggtgcgtctgactgcacgttggccgcatcttctgcatttg
cgggaatagcagagtggctgaaatcgaaaaggtcgccggggtgggag
agtagcccctgagtccagctcgggagcgcgtcgtgagagtttatgac
tggaggcgcatcgtgattgtcactttccggtgtagggattgttgcag
ctgaagttgatggttcactggtggttgttgcggtcctggctttcttt
gttctcgctctaggtgcagccccttttgccttggagcgttgggatga
gagccaattgaaggtacaatccttgttccaacgcttgcaatttgaac
acgaaacccagccgttttcattggcctcgtttctattttccggggca
tcacagcgtcgcttgcccttgcgacagggatcgcagctatgattctg
gcgtcggcgcgtatctgccatgctgtgtgtgcaggagaatatcgaca
gtgatatctgtatagatggt.

AlcA Promoter (5′ to the RNAi Construct)

(SEQ ID NO: 35)
Atgcatatgcgggatagttccgacctaggattggatgcatgcggaac
cgcacgagggcggggcggaaattgacacaccactcctctccacgcac
cgttcaagaggtacgcgtatagagccgtatagagcagagacggagca
ctttctggtactgtccgcacgggatgtccgcacggagagccacaaac
gagcggggccccgtacgtgctctcctaccccaggatcgcatccccgc
atagctgaacatctatataactgca.

Maize

In maize a gene on chromosome six was found that was highly homologous to the Arabidopsis SEX1 gene. The RNAi construct was designed using 371 bp of DNA upstream of the predicted TAG stop codon and 28 bp downstream of the TAG stop codon. The RNAi construct was placed behind the constitutively expressed ubiquitin promoter and used the same Pdk intron as in the Arabidopsis constructs. The RNAi inverted repeat was followed by an octopine synthase terminator.

RNAi Construction/Transformation

Arabidopsis

DNA for the RNAi inverted repeat against the Arabidopsis SEX1 and SEX4, the SAG12 promoter, the 35S promoter and the Alc promoter system and the 3′ OCS terminator were synthesized by Bio Basic Inc. (Markham Ontario, Canada) and placed in the pUC57 plasmid vector. The plasmid vector was amplified in competent E. coli strain DH5α (Invitrogen 18265-017). DH5α was transformed using a heat shock of 42° C. for 20 seconds. Plasmid was isolated using QIAprep miniprep kit (Quiagen, Valencia Calif.) according to the manufacturer's directions. Once a sufficient quantity of vector plasmid was obtained, promoter-RNAi-terminator constructs were assembled in the pUC57 vector. Two μg of DNA of the plasmid containing either the SEX1 or SEX4 targeted RNAi inverted repeat sequence and two μg of the vector containing either the SAG12 or Alc promoter each with the 3′OCS terminator were digested with two units each BbvCI and NcoI in buffer number four (New England Biolabs, Ipswich Mass., USA) for four hours at 37° C. The vector with promoter and RNAi inverted repeat were gel purified in 0.7% agarose and extracted using a MinElute gel extraction kit (Quiagen, Valencia Calif., USA) according to the manufacturer's directions. The RNAi inverted repeat was ligated into the vector between the promoter and terminator for 3 days at 4° C. using 1 unit T4 ligase (Invitrogen, Carlsbad Calif., USA). The ligation was done in a total volume of 10 μl using three times the amount of insert to vector. The fully assembled promoter-RNAi-terminator constructs were then placed in the pART27 destination vector (Gleave, 1992) for Arabidopsis transformation. Both vector and insert were digested with six units NotI in buffer number 3 (New England Biolabs, Ipswich Mass., USA) for three hours at 37° C. The pART27 vector was dephosphorylated using 10% v/v of 1 U/μl cow intestine alkaline phosphatase (CAP; Roch, Nutley N.J., USA) for 30 minutes at 37° C. The vector and inserts were gel purified in 0.7% agarose and extracted using a MinElute gel extraction kit (Quiagen, Valencia Calif., USA) according to the manufacturer's directions. The promoter-RNAi-terminator constructs were ligated into the destination vector for two days at 4° C. using one unit T4 ligase (Invitrogen, Carlsbad Calif., USA). The ligation was done in a total volume of 10 μl using three times the amount of insert to vector. Confirmation of the vector assembly detailed above was done by PCR using custom designed primers for the pART27 vector and different parts of the promoter RNAi construct (Table 1). 30 ng of vector with RNAi constructs was transformed into a 50 μl of a near saturated culture of Agrobacterium strain GV3101 bp electroporation.

TABLE 1
Primers used to confirm Arabidopsis
transgene insertion

| | | | Product |
|---|---|---|---|
| Transgenic | | | Size |
| Line | Primer | Sequence (5′-3′) | (kb) |
| Alc Empty | ALCR1 | CGTGGACCGTCGTTCTTATT | 1.4 |
| Vector | | (SEQ ID NO: 36) | |
| | P27R1 | ATACTTTCTCGGCAGGAGCA | |
| | | (SEQ ID NO: 37) | |
| | P57R1 | TGTGGAATTGTGAGCGGATA | 2.9 |
| | | (SEQ ID NO: 38) | |
| | ALCR2 | CGCGTCGTGAGAGTTTATGA | |
| | | (SEQ ID NO: 39) | |
| Alc GWD-RNAi | ALCR1 | CGTGGACCGTCGTTCTTATT | 1.4 |
| | | (SEQ ID NO: 40) | |
| | P27R1 | ATACTTTCTCGGCAGGAGCA | |
| | | (SEQ ID NO: 41) | |
| | ATS1R1 | TTCCTACACTGGGTCCCAAC | 2.2 |
| | | (SEQ ID NO: 42) | |
| | 35SR1 | GAAGCAAGCCTTGAATCGTC | |
| | | (SEQ ID NO: 43) | |
| Alc PGP-RNAi | ALCR1 | CGTGGACCGTCGTTCTTATT | 1.4 |
| | | (SEQ ID NO: 44) | |
| | P27R1 | ATACTTTCTCGGCAGGAGCA | |
| | | (SEQ ID NO: 45) | |
| | ATS4R1 | AATCTTCCCAGTCCCAACTGT | 2.2 |
| | | (SEQ ID NO: 46) | |
| | 35SR1 | GAAGCAAGCCTTGAATCGTC | |
| | | (SEQ ID NO: 47) | |
| SAG Empty | SAG12R1 | GATGTGAAGGAATCGCCCTA | 1.7 |
| Vector | | (SEQ ID NO: 48) | |
| | P27R1 | ATACTTTCTCGGCAGGAGCA | |
| | | (SEQ ID NO: 49) | |
| SAG GWD-RNAi | SAG12R1 | GATGTGAAGGAATCGCCCTA | 1.7 |
| | | (SEQ ID NO: 50) | |
| | P27R1 | ATACTTTCTCGGCAGGAGCA | |
| | | (SEQ ID NO: 51) | |
| | ATS1R1 | TTCCTACACTGGGTCCCAAC | 1.5 |
| | | (SEQ ID NO: 52) | |
| | SAG12R2 | CCGAGCAAAGTGAGTGAACA | |
| | | (SEQ ID NO: 53) | |
| SAG PGP-RNAi | SAG12R1 | GATGTGAAGGAATCGCCCTA | 1.7 |
| | | (SEQ ID NO: 54) | |
| | P27R1 | ATACTTTCTCGGCAGGAGCA | |
| | | (SEQ ID NO: 55) | |
| | ATS4R1 | AATCTTCCCAGTCCCAACTGT | 1.5 |
| | | (SEQ ID NO: 56) | |
| | SAG12R2 | CCGAGCAAAGTGAGTGAACA | |
| | | (SEQ ID NO: 57) | |

Agrobacterium was grown to near saturation in liquid culture containing 100 μg ml⁻¹ spectinomycin and 150 μg ml⁻¹ rifampicin. Arabidopsis thaliana Col-0 were transformed using the floral dip method (Clough and Bent, 1998) and six independent transformation events per construct were produced.

Primary transformants were selected by kanamycin resistance using a rapid selection method (Harrison et al., 2006). Briefly, seed from primary transformants were selected on plates containing 0.8% Phytoblend (Caisson Laboratories, North Logan Utah), 4.3 g/L MS salts (MSP C0130, Caisson Laboratories, North Logan Utah), 1% sucrose, 2.5 mM MES, and 50 μg/ml kanamycin using a rapid selection method to generate T₁ plants (Harrison et al. 2006). T₁ plants were grown on soil and allowed to self-pollinate. Approximately 100 seeds from T₁ plants were plated on kanamycin selection plates and those with a 3:1 ratio of resistant plants to susceptible plants were selected and grown to maturity on soil (T₂ generation). Approximately 100 seeds from each plant selected in the T₂ generation were planted and those plants with 100% resistance were selected and used for all further experiments (T₃ generation). Transgene insertion was confirmed by PCR using the primers in Table 1. Azygous plants were found by plating seeds from primary transformants on plates as above without kanamycin. Lack of transgene was confirmed by PCR.

Maize

The RNAi construct for maize was made as described for Arabidopsis. The pMCG1005 vector was used as the destination vector for maize transformation. This vector was provided by Dr. Heidi Kaeppler at the University of Wisconsin-Madison. Once constructs were completed the pMCG1005 vector with the RNAi cassette was isolated using QIAprep miniprep kit (Quiagen, Valencia Calif.) according to the manufacturer's directions. Confirmation of the vector assembly detailed above was done by PCR using custom designed primers for the pMCG1005 vector and different parts of our promoter RNAi construct (Table 2). Plasmid was then lyophilized and sent to Dr. Heidi Kaeppler's maize transformation facility. Transformation into line B73 was done using Agrobacterium and primary transformants were back-crossed to wild type B73 plants.

Progeny from this cross containing the transgene (T₁) were selected for when plants were four weeks old by painting the tip of third oldest leaf from the bottom with 0.1% w/v glufosinate. Glufosinate was prepared from commercially available Finale™ containing 11.33% glufosinate (Bayer Crop Science Monheim, Germany). After one week, plants were scored and resistant plants were confirmed to contain the gene construct using PCR and primers in Table 2.

TABLE 2
Primers used to confirm maize
transgene insertion

| | | | Product |
|---|---|---|---|
| Transgenic | | | Size |
| Line | Primer | Sequence (5′-3′) | (kb) |
| Ubi Empty | P1005F1 | GCGTTCAAAAGTCGCCTAAG | 1.4 |
| Vector | | (SEQ ID NO: 58) | |
| | UbiR1 | TGGACCACACGGTAATAGCA | |
| | | (SEQ ID NO: 59) | |
| Ubi SEX1 Like- | P1005F1 | GCGTTCAAAAGTCGCCTAAG | 1.4 |
| RNAi | | (SEQ ID NO: 60) | |
| | UbiR1 | TGGACCACACGGTAATAGCA | |
| | | (SEQ ID NO: 61) | |
| | P1005F1 | GCGTTCAAAAGTCGCCTAAG | 1.9 |
| | | (SEQ ID NO: 62) | |
| | ZMS1F1 | ATGAAGGAGACAAGCGTTGG | |
| | | (SEQ ID NO: 63) | |

### Plant Material and Growth Conditions

Arabidopsis

Wild type (Col-0), azygous, Sex1 KO line (SALK_{—}077211), Sex4 KO line (SALK_{—}102564), T3 homozygous lines containing the alcohol-inducible RNAi against the glucan water dikinase (GWD) or the phosphoglucan phosphatase, and T₃ homozygous empty vector lines containing the alcohol-inducible promoter but without the inverted repeat RNAi, as well as lines containing the senescence inducible RNAi against GWD and the corresponding knock out lines construct were used. Seeds were cold treated at 4° C. in distilled water for three days to ensure uniform germination. Seeds were then germinated and grown in 6 cm diameter plastic pots using Sun Gro Redi-earth plug and seedling mix (Sun Gro, Bellevue Wash., USA). Plants were watered with deionized water for the first 4 weeks and then quarter-strength Hoagland's solution thereafter. Plants were grown in a Percival AR4 growth chamber (Percival, Perry Iowa, USA) with a 12 hour photoperiod. Day temperature was 22° C. and night temperature was 18° C. The quantum flux, measured at leaf level, was 120 μmol m⁻²s⁻¹. Humidity was maintained at a minimum of 60% RH.

To induce alcohol-inducible lines, all plants were sprayed with 3% ethanol using a spray bottle obtained locally. Plants were sprayed once each day 4 hours before the lights went off in the growth chamber. Plant material for starch analysis was harvested when transitory starch was at a minimum in the morning just before the lights came on the growth chamber. Leaf material for transcript analysis was harvested in the evening just after the lights went off when SEX1 and SEX4 transcript levels have been found to be at their maximum (Smith et al. 2004). The senescence inducible plants were grown in conditions stated above for 40 days. After 40 days photographs were taken and leaves were stained with undiluted Lugal solution, Fluka 62650 (Sigma Aldrich, St. Louis Mo., USA) for starch in the morning.

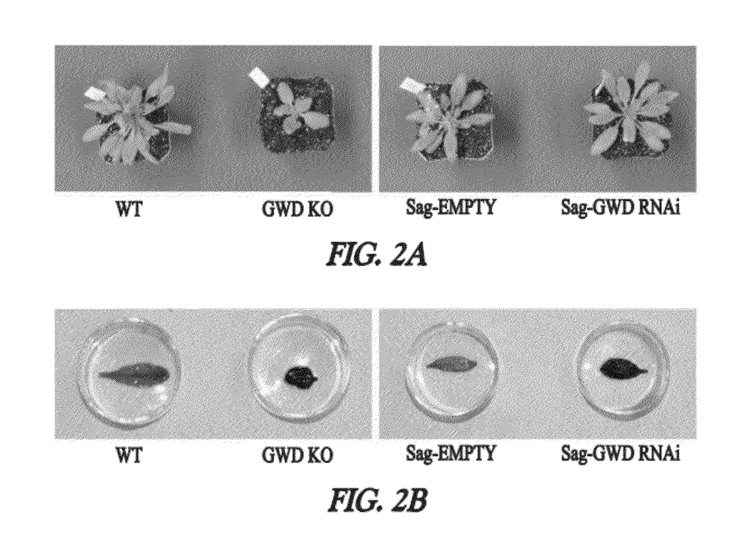

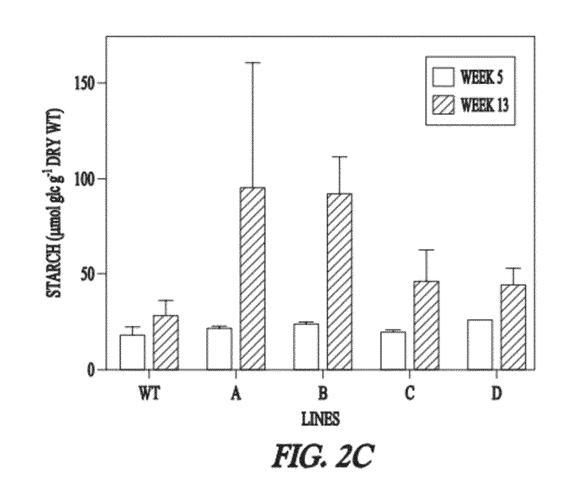

The senescence inducible plants were grown in conditions stated above for 40 days. After 40 days, photographs were taken and leaves were stained with undiluted Lugal solution, Fluka 6250 (Sigma Aldrich, St. Louis Mo., USA) for starch in the morning. (FIG. 2)

Maize

Seeds of T₁ maize lines containing RNAi construct against the SEX1 like gene and empty vector lines containing the ubiquitin promoter, but without the inverted repeat RNAi construct, were germinated and grown in 8 cm diameter plastic pots using Baccto professional planting mix (Michigan Peat Company, Houston Tex., USA). When plants were approximately six weeks old they were transferred to 16 cm diameter plastic pots. Plants were grown in a Bigfoot GC-20 growth chamber (Biochambers Inc, Winnipeg, Canada) lit by fluorescent tubes until they were 9 weeks old. Plants were then potted into larger 25 cm diameter plastic pots and moved to a greenhouse on Michigan State University's campus in East Lansing Mich. during the months of July, August, and September. Plants were watered with tap water containing Miracle-Gro™ tomato plant food 18-18-21 (Scotts Miracle-Gro Products Inc. Marysville Ohio, USA) at the manufacturer's recommended concentration of 2.5 ml per 4 liters of water. Plant material for starch analysis was harvested in the morning for both growth chamber and greenhouse grown plants. For plants in the growth chamber this was just before the lights came on. For plants in the greenhouse this was about four hours after sunrise. Plant material for transcript analysis was harvested in the evening just after the lights went out in the growth chamber or about an hour after sunset for greenhouse grown plants.

### Starch Analysis

Harvested leaf material was placed in pre-weighed two ml microfuge tubes or 50 ml plastic tubes depending on the amount of tissue being harvested. Tubes were then quickly weighed to obtain a fresh weight. Tubes were opened and placed in a drying oven at 70° C. overnight. Tubes were weighed again after drying to obtain a dry weight. If material was harvested in a 50 ml tube one glass marble was placed in the tube and shaken to break up material. A known amount of plant material was then sub aliquoted into a 2 ml tube to obtain a smaller representative sample. One 4 mm silicon carbide particle, #6 grit, (11079140sc BioSpec Inc, Bartlesville Okla., USA) was placed in the 2 ml tube. Leaf material was ground at room temperature in a Retsch MM301 ball mill (Retsch, Newtown Pa., USA) at a frequency of 30 for 30 seconds using a 24 position Qiagen tissue lyser adapter 69982 (Quiagen, Valencia Calif., USA).

To denature enzymes and remove soluble carbohydrates one ml of 80% ethanol was added to ground leaf material and incubated at 80° C. for 20 minutes. Following incubation samples were centrifuged at 20,000 g for five minutes. The supernatant was discarded and the ethanol incubation was repeated. After the second ethanol incubation the sample was washed with ethanol up to three additional times to remove color. Following the ethanol wash the tubes with pellets were placed in a speed vac at low heat for 30 minutes to remove residual alcohol. The pellet was then resuspended in 250-1000 μl of 200 mM KOH and incubated in a dry bath with tube locks at 95° C. for 30 minutes to gelatinize the starch. The tubes were then allowed to cool at room temperature for five minutes and 1 M acetic acid was added to each tube to bring the pH to 5. Starch in the sample was broken down to glucose by adding 5 μl of an enzyme cocktail containing 5 units aamylase (E-ANAAM Megazyme, Bray, Wicklow, Ireland) and 6.6 units amylogucosidase (E-AMGDF Megazyme, bray, Wicklow, Ireland) in 200 mM sodium acetate pH 4.8. Samples were incubated at room temperature with starch degrading enzymes for two days. Following starch digestion samples were centrifuged at 20,000 g for at 4° C. for 20 minutes and the supernatant was transferred to a fresh microfuge tube.

The resulting glucose was assessed on a 96 well plate in a Spectra Max M2 plate reader (MDS Analytical Technologies, Sunnyvale Calif., USA) at 340 nm using an NADP(H) linked assay. Each well that was used was filled with 200 μl of 150 mM Hepes buffer pH 7.2 containing 15 mM MgCl₂, 3 mM EDTA, 500 nmol NADP, 500 nmol ATP and 0.4 units glucose-6-phosphate dehydrogenase (G8529 Sigma St. Louis Mo., USA). Five μl of sample was added to each well and the reaction was started by adding 0.5 units of hexokinase (H4502 Sigma St. Louis Mo., USA). Because the Spectra Max M2 plate reader can also determine the path length of aqueous samples absorbance units were normalized to a 1 cm path length and absolute glucose amounts were determined using an extinction coefficient of 6220 L mol⁻¹ cm⁻¹ for NADPH at 340 nm (Lowry & Passonneau 1972).

### Transcript Analysis

RNA was extracted using Qiagen RNeasy Plant Mini Kit (74904 Qiagen, Valencia Calif., USA) according to manufacturer's directions. Once RNA was isolated cDNA was synthesized using 300 ng of total RNA from Arabidopsis or 500 ng of RNA from maize. Super Script II reverse transcriptase (18064, Invitrogen, Carlsbad Calif., USA) was used according to manufactures directions. cDNA was stored at −80° C. until used for qPCR.

For Arabidopsis two μl of cDNA was diluted into 198 μl of RNase free water and two μl of the resulting dilution were used for qPCR analysis. For maize two μl of resulting cDNA was used directly. An Eppendorf Mastercycler ep Realplex qPCR thermocycler with a 96 position silver block with SYBR green PCR master mix (4309155 Applied Biosystems, Carlsbad Calif. USA) was used according to the manufactures directions. The thermal profile was: 95° C. for 10 min; 40 cycles of 95° C. for 15 sec, and 60° C. for 1 min. This was followed by a melting curve. Transcript amounts were normalized using the Actin2 housekeeping gene transcript, At3G18780 for Arabidopsis or the Alpha Actin housekeeping gene from maize. All primers used in qPCR reactions were used at a concentration of 0.5 μM in the PCR tube. Sequences for primers used are listed in Tables 3 and 5. A slightly larger fragment of each target sequence had previously been amplified from a reverse-transcribed RNA extract, and the resulting DNA was quantitated. Dilutions of these, containing known numbers of copies of the target sequences, were used to prepare standard curves that were used to determine the copy numbers of the plant samples. Primer sequences used to generate templates used in quantification are listed in Tables 4 and 6.

TABLE 3
Primers used for Arabisopsis qPCR

| | Forward/ | | Product |
|---|---|---|---|
| Gene Target | Reverse | Sequence (5′-3′) | Size (bp) |
| ACT2 | Forward | CAAAGGCCAACAGAGAGAAGA | 137 |
| At3g18780 | | (SEQ ID NO: 64) | |
| | Reverse | ATCACCAGAATCCAGCACAA | |
| | | (SEQ ID NO: 65) | |
| SEX1 | Forward | GCGGTGAACGATAAATTGCT | 191 |
| At1g10760 | | (SEQ ID NO: 66) | |
| | Reverse | GCTTGCTCCCATCTCTGTTC | |
| | | (SEQ ID NO: 67) | |
| Sex4 | Forward | GCTAATATCGCCTCCGATCA | 110 |
| At3g52180 | | (SEQ ID NO: 68) | |
| | Reverse | CTTCATCCTTTGCAACACCA | |
| | | (SEQ ID NO: 69) | |

TABLE 4
Primers used for Arabidopsis template
generation for qPCR standards

| | Forward/ | | Product |
|---|---|---|---|
| Gene Target | Reverse | Sequence (5′-3′) | Size (bp) |
| ACT2 | Forward | GGTGATGAAGCACAATCCAA | 397 |
| At3g18780 | | (SEQ ID NO: 70) | |
| | Reverse | CAGTAAGGTCACGTCCAGCA | |
| | | (SEQ ID NO: 71) | |
| SEX1 | Forward | TGCCATTTGGTGTTTTTGAG | 477 |
| At1g10760 | | (SEQ ID NO: 72) | |
| | Reverse | GACCGGGATATGCTCCTACA | |
| | | (SEQ ID NO: 73) | |
| Sex4 | Forward | ATTCGGGTGCATTCAAAGAG | 397 |
| At3g52180 | | (SEQ ID NO: 74) | |
| | Reverse | GGCTTGGATGCTGCTTATGT | |
| | | (SEQ ID NO: 75) | |

TABLE 5
Primers used for maize qPCR

| | Forward/ | | Product |
|---|---|---|---|
| Gene Target | Reverse | Sequence (5′-3′) | Size (bp) |
| Alpha Actin | Forward | CCGAAAATGCTTCTGAGCTT | 147 |
| | | (SEQ ID NO: 76) | |
| | Reverse | GCATGCGCTTAGTTGGGTAT | |
| | | (SEQ ID NO: 77) | |
| Homolog | Forward | TGGTCGTGCTATGAGCTTTG | 116 |
| SEX1 | | (SEQ ID NO: 78) | |
| | Reverse | CAAAGCTCATAGCACGACCA | |
| | | (SEQ ID NO: 79) | |

TABLE 6
Primers used for maize template
generation for qPCR standards

| | Forward/ | | Product |
|---|---|---|---|
| Gene Target | Reverse | Sequence (5′-3′) | Size (bp) |
| Alpha Actin | Forward | CTGTACGGGAACATCGTCCT | 415 |
| | | (SEQ ID NO: 80) | |
| | Reverse | GCATCTGAATCACGAAGCAGG | |
| | | (SEQ ID NO: 81) | |
| Homolog | Forward | ATGAAGGAGACAAGCGTTGG | 417 |
| SEX1 | | (SEQ ID NO: 82) | |
| | Reverse | CTGCTCCAGCATAACCTTCC | |
| | | (SEQ ID NO: 83) | |

### Microscopy

Leaf material from maize RNAi lines was taken from fully mature 9 week old plants growing in a greenhouse at Michigan State University. Fully expanded leaves from the top, fully lit, part of the plant were chosen. Leaf sections 1-2 mm by 10 mm were placed in a seven ml vial with 2 ml of 2.5% glutaraldehyde/formaldehyde in 100 mM cacodylate buffer. Microwave assisted fixation was done with an EMS-9000 precision pulsed laboratory microwave oven (Electron Microscopy Sciences, Hatfield Pa., USA).

Samples were fixed for 2 minutes at 15% power and a temperature of 30° C. Post fixation was done in two ml of 1% OsO4 in 100 mM cacodylate buffer. Samples were post fixed for ten minutes at 15% power and a temperature of 30° C. Following post fixation samples were dehydrated in the microwave at 15% power at 30° C. for 10 minutes at each step using a progressively more concentrated acetone series of 50%, 70%, 80%, 90%, and 100%. Dehydrated samples were fixed in EPON resin in the microwave at 15% power at 45° C. for 20 minutes at each step using an EPON:acetone serious of 1:1, 3:1, 100%. The embedded samples were placed in molds and polymerized at 60° C. overnight. Samples were sectioned using a microtome and diamond knife to a thickness of 500 nm. Sections were transferred to a glass microscope slide and stained for five minutes using a quarter strength Lugols solution (Fluka 62650, Sigma-Aldrich, St. Louis Mo., USA). Sections were viewed and photographed using a standard bright field light microscope.

### Results

It was demonstrated in herein that plants that are lacking the glucan water dikinase enzyme (GWD KO) have high starch levels throughout their life which results in a significant growth penalty. However, plants with the senescence induced RNAi gene against GWD does not exhibit a decrease in growth and after 2 months contains high leaf starch levels comparable to the GWD KO line. See FIGS. 2A-B.

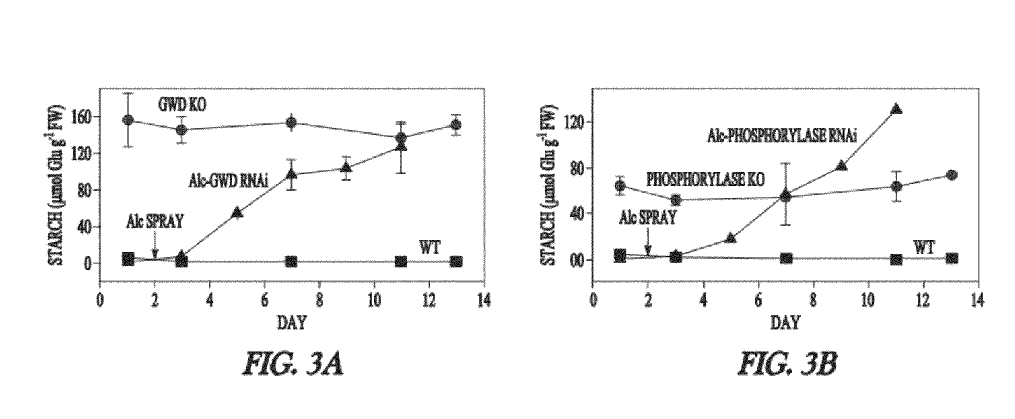

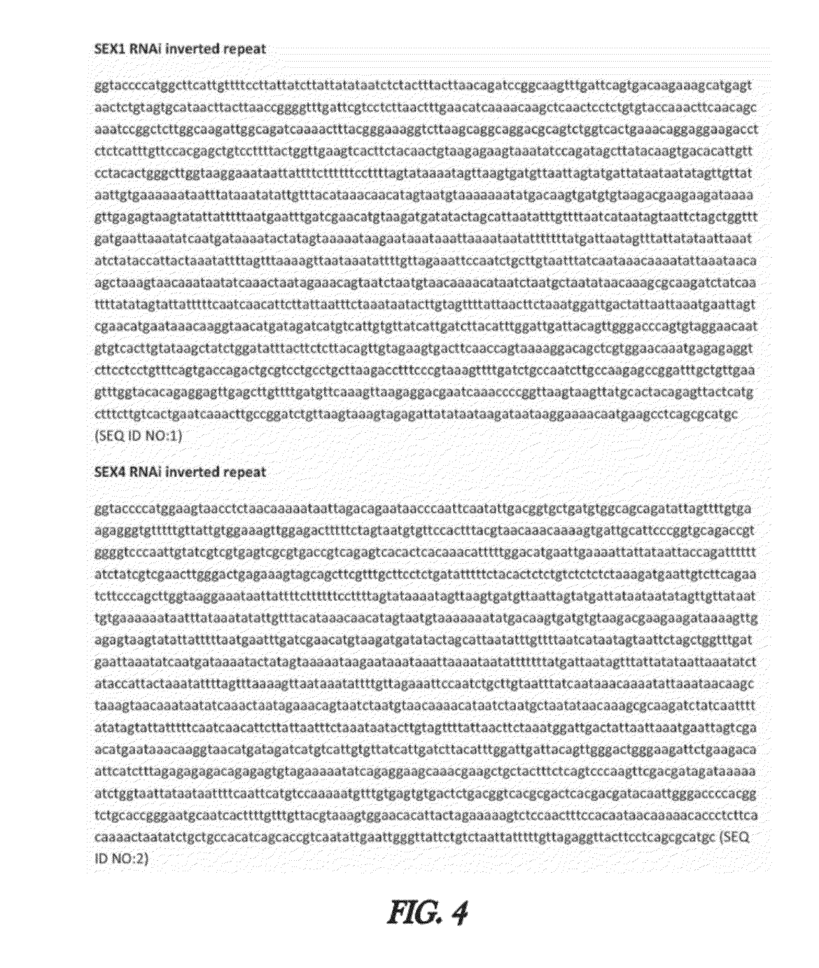

Wild type, primary transformant plants containing alcohol inducible RNAi against the glucan water dikinase (GWD) or the phosphoglucan phosphatase enzyme, both involved in leaf starch degradation, and the corresponding knock out lines (plants that lack enzyme throughout their lives) were grown for 27 days. Alcohol inducible plants were then sprayed with 2% ethanol. Leaves were taken for starch determinations in the morning. The alcohol (Alc) inducible plants quickly accumulated as much starch as the knock out line. In the case of the Alc inducible RNAi against the phosphoglucan phosphatase, the starch levels exceeded those of the knock out line. See FIG. 3.

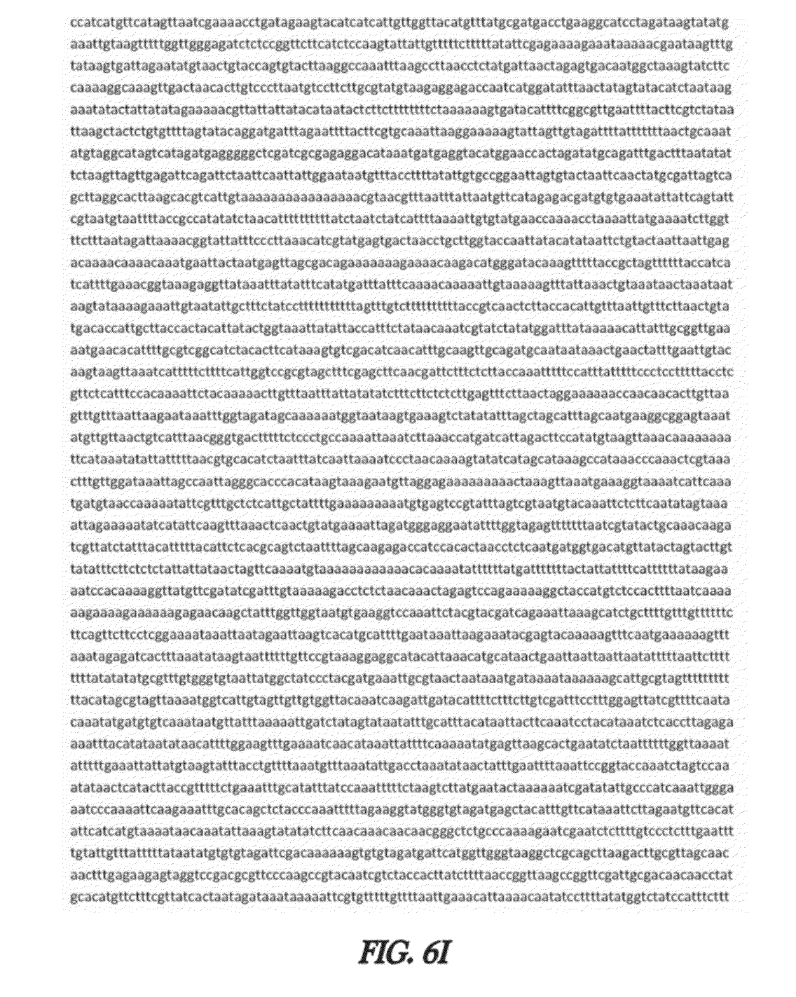

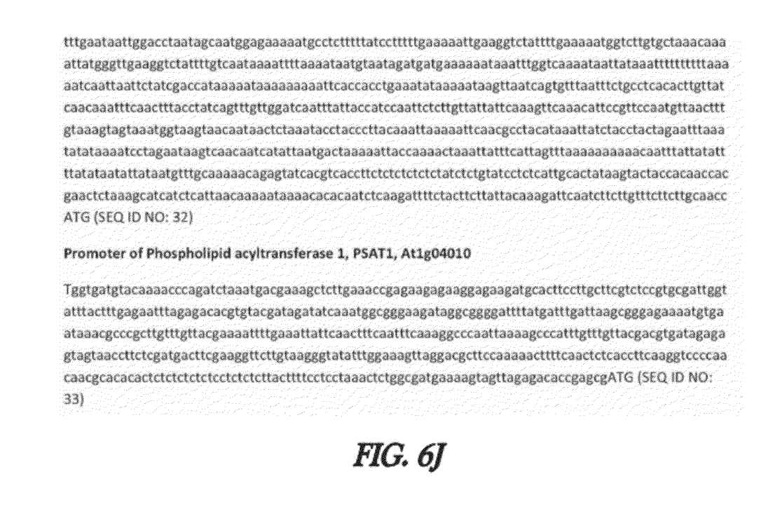

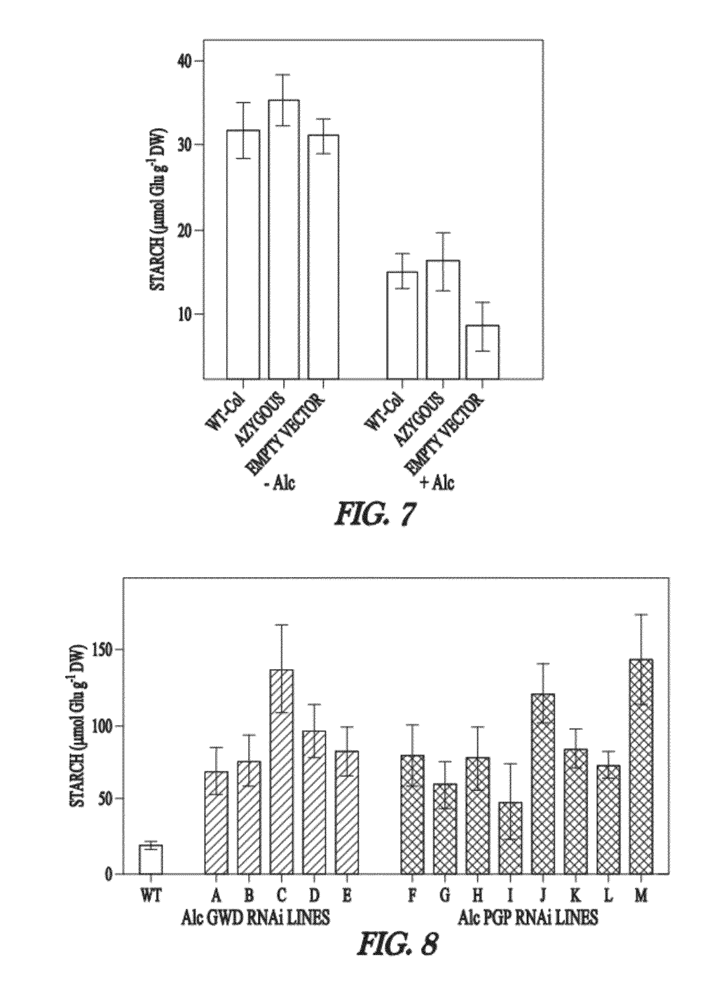

Additional data with the alcohol inducible promoter was also obtained. Two groups of wild type, azygous and empty vector lines containing only the alcohol inducible promoter were grown for four weeks without ethanol. The first group was immediately assayed for starch. The second group was grown an additional week while being sprayed daily with 3% ethanol. All lines showed similar levels of starch with and without ethanol (FIG. 7).

Wild type and transgenic lines containing the alcohol inducible RNAi against the gene for the glucan water dikinase or the phosphoglucan phosphatase were grown for eight weeks. Plants were then sprayed for one week with 3% v/v ethanol. All lines accumulated more starch than WT (FIG. 8). The highest starch accumulating lines accumulated six times more starch than the WT. Equal levels of starch were seen in both the RNAi lines against GWD and the RNAi lines against PGP. This is in contrast to starch levels in the corresponding KO mutants where GWD KO lines accumulate twice as much starch as PGP KO lines (Messerli, Nia, Trevisan, Kolbe, Schauer, Geigenberger, Chen, Davison, Fernie, & Zeeman 2007).

Transcript levels were examined in the higher starch accumulating lines by qRT-PCR. Leaf material for transcript analysis was taken the night after leaf material was taken for starch determinations shown in Figure*. Transcripts for the gene encoding the GWD or PGP were reduced by approximately 50% in all alcohol inducible transgenic lines tested (FIG. 9).

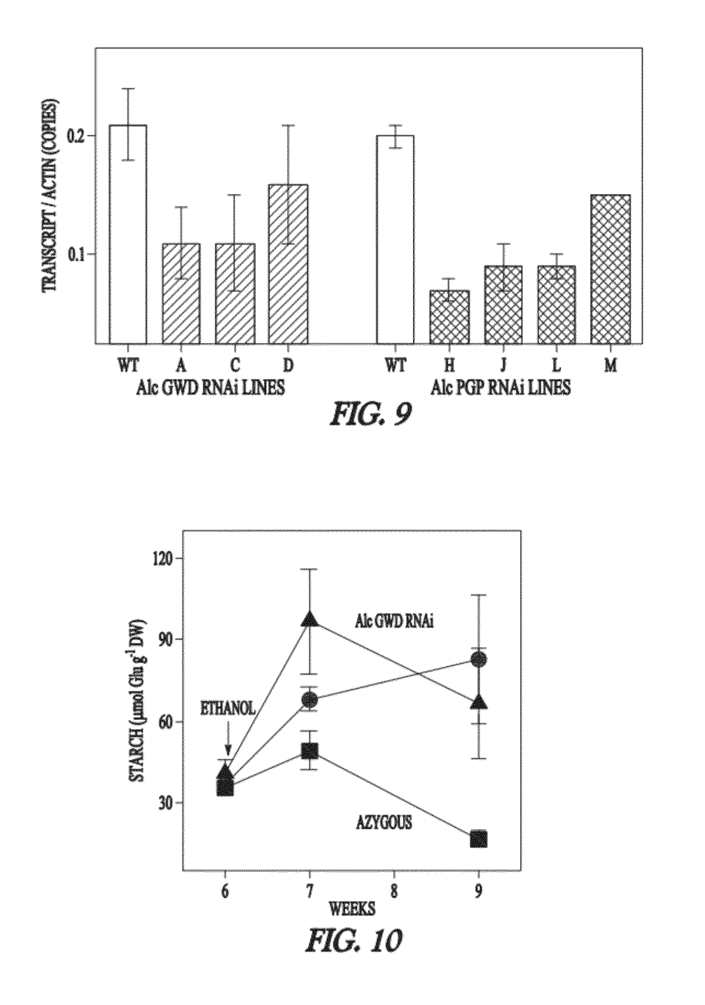

A time course of starch accumulation was conducted on two of the higher starch accumulating lines. Plants were allowed to grow for six weeks and then samples were taken weekly in the morning. Starch levels were low in both the azygous control line and the two transgenic RNAi lines tested before plants were sprayed with ethanol. Once spraying with ethanol commenced starch levels rose rapidly in the two transgenic lines. Starch levels in the azygous control plants remained low (FIG. 10).

Because plants that accumulate large amounts of starch often exhibit a strong yield penalty in terms of overall biomass whether this was occurring in the transgenic RNAi lines was investigated. An azygous control line, two transgenic RNAi lines against the GWD gene and tDNA KO plants of the GWD gene were grown for three weeks. Plants were then sprayed with 3% ethanol daily and whole plants were harvested weekly during the exponential phase of growth and dried to determine total dry biomass. Growth rates of azygous and transgenic lines were similar; however the Sex1 KO plants grew much slower (FIG. 11).

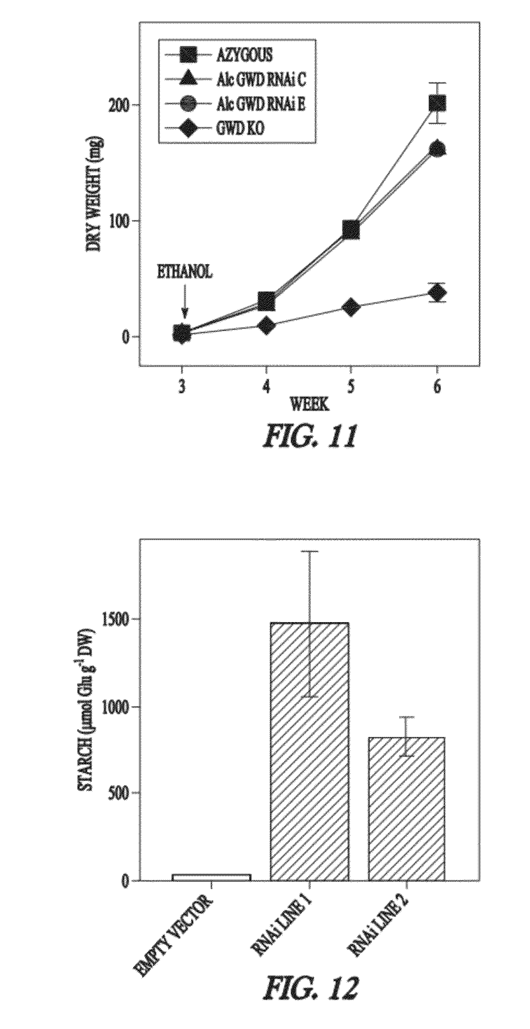

The maize EST database (ncbi.org) was searched using the last 500 bp of the Arabidopsis SEX1 cDNA sequence. A region on an EST, GenBank accession CD973834, was found that that was 74% identical using 98% of the query sequence. An RNAi construct was made using 400 bp of sequence that span from position 143 to 542 on the EST. T his 400 bp of sequence aligns to three regions in the maizeGDB.org database B73 RefGen_v1 located on chromosome six between positions 107665607 and 107666414. The RNAi construct was placed behind the ubiquitin promoter. Two RNAi lines against the SEX1 like gene in maize were grown in a growth chamber and a 12 mm dia leaf punch was taken from the fourth oldest leaf (from the bottom) when the plants were six weeks old. Starch levels were elevated 22-42 fold over the empty vector control line (FIG. 12). This is a starch accumulation of 15%-26% by weight.

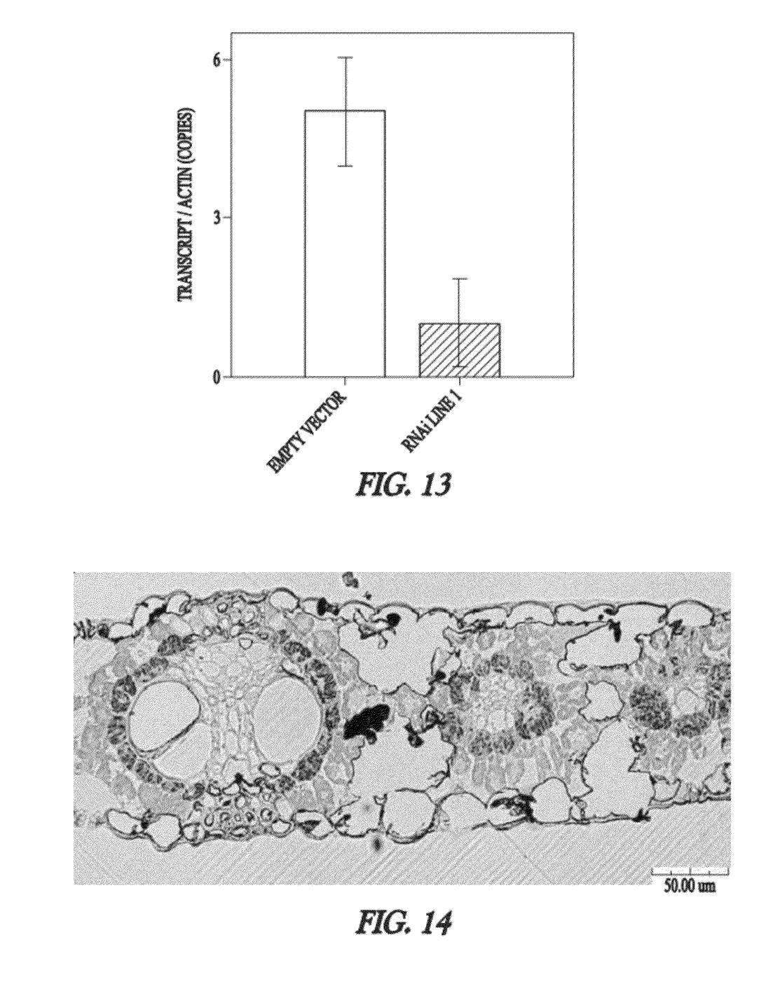

Transcript levels were examined in the highest starch accumulating lines by qRT-PCR using the primers listed in Table 5. Leaf material for transcript analysis was taken the night after leaf material was taken for starch determinations shown in FIG. 12. Transcript for the SEX1 like gene was reduced by 80% in the RNAi line (FIG. 13).

Microscopy was done in order to determine where the starch accumulates in the maize RNAi lines. Leaf material was taken from the top fully expanded leaves from nine week old plants growing in a greenhouse. Leaf material was taken approximately six hours after sunrise. After plants were fixed, embedded, and sectioned, sections were stained using a quarter strength Lugol solution and photographed using standard light microscopy. Starch accumulation was found exclusively in the bundle sheath cells (FIG. 14).

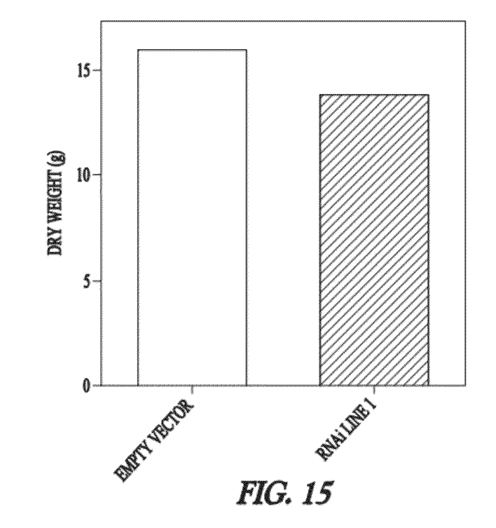

No qualitative differences in growth rates or overall biomass in empty vector control lines and RNAi lines were observed. Total above ground biomass was measured in nine week old plants that were grown in growth chambers. We found no differences in fresh (data not shown) or dry weight between the control line and RNAi line (FIG. 15).

### Discussion

The experiments demonstrate that leaf starch can accumulate without incurring a yield penalty. While not wanting to be limited to any particular theory, there are at least two explanations as to why no yield penalty was observed in the transgenic starch accumulating plants. The first, because a later onset of starch accumulation may have allowed exponential growth to take place before starch accumulation and concomitant night starvation occurs (Usadel et al. 2008). The second is that the RNAi did not completely block starch degradation, allowing some carbon to be degraded, sensed, and used at night. The mild SEX4 KO phenotype supports this idea. SEX4 KO plants accumulate 50% of the starch that SEX1 KO accumulate and a diel synthesis and degradation of starch is still observed. SEX4 KO plants are about the same size as WT. The small phenotype of other starch accumulating mutants such as the maltose transporter MEX1 KO or the cytosolic amylomaltase DPE2 KO which accumulate less starch then SEX4 KO would seem to run counter to this theory. However, both the maltose transporter and the cytosolic amylomaltase are further downstream in the starch degradation pathway than the glucan water dikinase or phosphoglucan phosphatase and a knockout of enzymes further downstream would allow a buildup of maltose and glucose both of which are reducing sugars and both may be sensed by signaling proteins (Rolland et al. 2006). The dwarf phenotype of these mutants may be more a result of maltose and/or glucose toxicity and less a result of absolute carbon starvation from starch accumulation. The transgenic maize plants also support this hypothesis. In the maize RNAi lines, the RNAi construct is behind the ubiquitin promoter and is therefore expressed during entire life cycle. Leaf starch levels were similar to that of the SEX1 KO in Arabidopsis, yet no yield penalty was observed. The situation in the maize RNAi lines could be similar to the SEX4 KO in Arabidopsis in that enough carbon can get through to sustain metabolism or at least satisfy sensing mechanisms. Twenty percent of the empty vector control line transcript level for the SEX1 like gene is still present in the RNAi line, so it is not a complete block.

The use of the alcohol inducible promoter system allowed for the demonstration that one can accumulate large amounts of leaf starch, however use of such a system may not be practical for the agronomic production of leaf starch. While the amount of ethanol needed to induce plants is low, the constant (e.g., daily) need to reinduce plants could be prohibitive in terms of labor and cost. A more practical solution would be the use of an inducible promoter triggered by an endogenous signal that is turned on at an appropriate time. The promoter of the Senescence Associated Gene 12 (SAG12), a cysteine protease, is activated only by senescence and drives expression late in the life cycle of the plant (Noh & Amasino 1999). It had been shown that when the SAG12 promoter was used in tomato to drive cytokinin biosynthesis leaf senescence was delayed (Swartzberg et al. 2006). A promoter such as SAG29 with stronger expression earlier in the development of the leaf may also be appropriate (Quirino et al. 1999).

Previous work in Dr. DellaPenna's laboratory found that the alcohol inducible promoter works poorly in maize (personal communication). A senescence inducible promoter has not yet been described in maize. Although the SAG12 promoter has been used in maize, the work with Arabidopsis indicated that it may induce expression too late in the life cycle of the plant. Therefore, the RNAi construct was placed behind the constitutive ubiquitin promoter. It was determined that starch accumulated to high levels in the transgenic RNAi lines, but starch accumulation was limited to the bundle sheath cells. This is different than what has been observed in other starch accumulating mutants of maize. All previously reported maize leaf starch accumulating mutants had defects in carbohydrate export. In the case of tdy1 phloem, loading is disrupted and in the case of sed1, plasmodesmata are modified disrupting sucrose transport (Russin et al. 1996; Ma et al. 2009). Both tdy1 and sed1 have a dwarf phenotype and leaf regions in which carbohydrate transport is altered are chlorotic or accumulate high levels of anthrocyanins. The high level of starch and it's localization to the bundle sheath cells in the instant RNAi line suggests that the SEX1 like sequence of DNA that was targeted encodes for a glucan water dikinase. The three regions in the maizeGDB.org database B73 RefGen_v1 that the RNAi sequence aligns to are not annotated as being a gene but lie between two regions annotated as genes. The dwarfing and severity of phenotypes observed in the tdy1 and sed1 mutants may be due to the fact that carbohydrate metabolism is blocked much further downstream allowing reducing sugars to build up causing sugar toxicity or sensing issues. The high starch and lack of phenotype observed in both Arabidopsis and maize RNAi lines suggests that when engineering plants for elevated leaf starch targeting the very beginning of the transitory starch degradation pathway is preferable and can avoid secondary effects from free sugar accumulation on yield and plant health.

It was determined that starch accumulated as a result of the suppression of GWD expression by RNAi is able to be broken down once alcohol induction stops (data not shown). This lends evidence that GWD works at the granule surface and is able to act upon previously under-phosphorylated starch allowing it to be broken down (Ritte et al. 2004). Control of GWD has important implications beyond biofuels. The phosphate content of starch is important for many industrial applications and those applications are engineered to use grain starch with no phosphate. By engineering GWD one can turn leaf starch into a commodity more akin to grain starch; this will allow a further diversification of this commodity in a volatile biofuels marketplace.

### BIBLIOGRAPHY

Clough S J, Bent A F (1998) Floral dip: a simplified method for Agrobacterium-mediated transformation of Arabidopsis thaliana. Plant J 16: 735-743.
Felenbok B (1991) The ethanol utilization regulon of Aspergillus nidulans: the alcA-alcR system as a tool for the expression of recombinant proteins. J Biotechnol 17: 11-18.
Gan S, Amasino R M (1995) Inhibition of leaf senescence by autoregulated production of cytokinin Science 270: 1986-1988.
Gleave A P (1992) A versatile binary vector system with a T-DNA organisational structure conducive to efficient intergration of cloned DNA into the plant genome. Plant Molecular Biology 20: 1203-1207.
Harrison S J, Mott E K, Parsley K, Aspinall S, Gray J C, Cottage A C (2006) A rapid and robust method of identifying transformed Arabidopsis thaliana seedlings following floral dip transformation. Plant Methods 2: 19.
Kötting O, Santelia D, Edner C, Eicke S, Marthaler T, Gentry M S, Comparot-Moss S, Chen J, Smith A M, Steup M, Ritte G, Zeeman S (2009) Starch Excess 4 is a laforin-like phosphoglucan phosphatase required for starch degradation in Arabidopsis thaliana. The Plant Cell 21: 334-346.
Lowry O H, Passonneau J V (1972) A flexible system of enzymatic analysis. Academic Press, Orlando, pp 1-291.
Ma Y, Slewinski T L, Baker R F, Braun D M (2009) Tie-dyed1 encodes a novel, phloem-expressed transmembrane protein that functions in carbohydrate partitionin Plant Physiol. 149: 181-194.
Messerli G, Nia V P, Trevisan M, Kolbe A, Schauer N, Geigenberger P, Chen J, Davison A C, Fernie A R, Zeeman S (2007) Rapid classification of phenotypic mutants of Arabidopsis via metabolite fingerprinting. Plant Physiol. 143: 1484-1492.
Noh Y-S, Amasino R M (1999) Identification of a promoter region responsible for the senescence-specific expression of SAG12. Plant Molecular Biology 41: 181-194.
Ohlrogge J, Allen D, Berguson B, DellaPenna D, Shachar-Hill Y, Stymne S (2009) Driving on biomass. Science 324: 1019-1020.
Quirino B F, Normanly J, Amasino R M (1999) Diverse range of gene activity during Arabidopsis thaliana leaf senescence includes pathogen-independent induction of defense-related genes. Plant Molecular Biology 40: 267-278.
Ritte G, Lloyd J R, Eckermann N, Rottmann A, Kossmann J, Steup M (2002) The starch-related R1 protein is an α-glucan, water dikinase. Proc. Natl. Acad. Sci. USA 99: 7166-7171.
Ritte G, Scharf A, Eckermann N, Haebel S, Steup M (2004) Phosphorylation of transitory starch is increased during degradation. Plant Physiol. 135: 2068-2077.
Rolland F, Sheen J, Sheen J (2006) Sugar sensing and signaling in plants: Conserved and novel mechanisms. Annu. Rev. Plant Biol. 57: 675-709.
Russin W A, Evert R F, Vanderveer P J, Sharkey T D, Briggs S P (1996) Modification of a specific class of plasmodesmata and loss of sucrose export ability in the sucrose export defectivel maize mutant. Plant Cell 8: 645-658.
Smith S M, Fulton D C, Chia T, Thorneycroft D, Chapple A, Dunstan H, Hylton C, Zeeman S C, Smith A M (2004) Diurnal changes in the transcriptome encoding enzymes of starch metabolism provide evidence for both transcriptional and posttranscriptional regulation of starch metabolism in arabidopsis leaves. Plant Physiol. 136: 2687-2699.
Swartzberg D, Dai N, Gan S, Amasino R, Granot D (2006) Effects of cytokinin production under two SAG promoters on senescence and development of tomato plants. Plant Biol. 8: 579-586.
Usadel B, Biasing O E, Gibon Y, Retzlaff K, Hohne M, Gunther M, Stitt M (2008) Global transcript levels respond to small changes of the carbon status during progressive exhaustion of carbohydrates in Arabidopsis rosettes. Plant Physiol. 146: 1834-1861.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method to accumulate starch in a plant without decreasing plant growth or yield comprising inhibiting expression of a protein or a gene coding for a protein that aids in starch degradation, wherein expression of the protein or the gene coding for the protein that aids in starch degradation is inhibited by expression of a heterologous polynucleotide so as to accumulate starch in a plant without a significant decrease in yield or plant growth.

2. The method of claim 1, wherein the heterologous polynucleotide codes for an RNA sequence which upon expression in the plant leads to a reduction in expression of the protein or a gene coding for the protein that aids in starch degradation.

3. The method of claim 1, wherein the heterologous polynucleotide comprises an inducible promoter, a promoter that drives expression at senescence, or a constitutive promoter.

4. The method of claim 3, wherein the promoter is an alcohol-inducible promoter or a senescence-induced promoter.

5. The method of claim 1, wherein the protein is glucan water dikinase, phosphoglucan phosphatase or β-amylase.

6. The method of claim 1, wherein expression is inhibited by RNA interference (RNAi).

7. The method of claim 1, wherein the heterologous polynucleotide comprises a nucleotide sequence that is antisense to at least a portion of a nucleotide sequence that codes for the protein that aids in starch degradation.

8. The method of claim 1, wherein the plant is a monocot.

9. The method of claim 1, wherein the plant is a dicot.

10. A method for preparing a plant that accumulates starch without decreasing plant growth or yield comprising introducing into the plant a heterologous polynucleotide that encodes an RNA sequence which upon expression in a plant leads to a reduction in expression of a protein or a gene coding for a protein that aids in starch degradation, wherein the polynucleotide is integrated into the genome of a plant cell and an intact plant is generated from the plant cell and reduction in expression of the protein or the gene coding for the protein that aids in starch degradation occurs without a significant decrease in yield or plant growth.

11. The method of claim 10, wherein the heterologous polynucleotide comprises an inducible promoter, a promoter that drives expression at senescence or a constitutive promoter.

12. The method of claim 11, wherein the promoter is an alcohol-inducible promoter or a senescence-induced promoter.

13. The method of claim 10, wherein the protein is glucan water dikinase, phosphoglucan phosphatase or β-amylase.

14. The method of claim 10, wherein expression is inhibited by RNA interference (RNAi).

15. The method of claim 10, wherein the heterologous polynucleotide comprises a nucleotide sequence that is antisense to at least a portion of a nucleotide sequence that codes for a protein that aids in starch degradation.

16. The method of claim 10, wherein the plant is a monocot.

17. The method of claim 10, wherein the plant is a dicot.

18. A transgenic plant or plant material that comprises a heterologous polynucleotide that encodes an RNA sequence which upon expression in a plant leads to a reduction in expression of a protein or a gene coding for a protein that aids in starch degradation, wherein reduction in expression of the protein or the gene coding for the protein that aids in starch degradation occurs without a significant decrease in yield or plant growth.

19. A method to prepare animal feed silage comprising producing animal feed silage from the transgenic plant or plant material of claim 18.

20. A method to prepare bio-fuel comprising producing biofuel from the transgenic plant or plant material of claim 18.

21. A method to accumulate starch in a plant without decreasing plant growth or yield comprising inhibiting endogenous expression of a protein or a gene coding for a protein that aids in starch degradation throughout the life of the plant, wherein the plant is complimented with a transgene coding for the protein which was inhibited which is operably linked to a promoter which is only active early in the life cycle of the plant so as to accumulate starch in the plant without a significant decrease in yield or plant growth.


SEQUENCE LISTING (84 sequences)

SEQ ID NO: 1 - DNA, 1604 bp, Artificial Sequence
  A synthetic oligonucleotide
ggtaccccatggcttcattgttttccttattatcttattatataatctctactttacttaacagatccggcaagtttgattcagtgacaagaaagcatgagtaactctgtagtgcataacttacttaaccggggtttgattcgtcctcttaactttgaacatcaaaacaagctcaactcctctgtgtaccaaacttcaacagcaaatccggctcttggcaagattggcagatcaaaactttacgggaaaggtcttaagcaggcaggacgcagtctggtcactgaaacaggaggaagacctctctcatttgttccacgagctgtccttttactggttgaagtcacttctacaactgtaagagaagtaaatatccagatagcttatacaagtgacacattgttcctacactgggcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcaattttatatagtattatttttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggacccagtgtaggaacaatgtgtcacttgtataagctatctggatatttacttctcttacagttgtagaagtgacttcaaccagtaaaaggacagctcgtggaacaaatgagagaggtcttcctcctgtttcagtgaccagactgcgtcctgcctgcttaagacctttcccgtaaagttttgatctgccaatcttgccaagagccggatttgctgttgaagtttggtacacagaggagttgagcttgttttgatgttcaaagttaagaggacgaatcaaaccccggttaagtaagttatgcactacagagttactcatgctttcttgtcactgaatcaaacttgccggatctgttaagtaaagtagagattatataataagataataaggaaaacaatgaagcctcagcgcatgc

SEQ ID NO: 2 - DNA, 1604 bp, Artificial Sequence
  A synthetic oligonucleotide
ggtaccccatggaagtaacctctaacaaaaataattagacagaataacccaattcaatattgacggtgctgatgtggcagcagatattagttttgtgaagagggtgtttttgttattgtggaaagttggagactttttctagtaatgtgttccactttacgtaacaaacaaaagtgattgcattcccggtgcagaccgtggggtcccaattgtatcgtcgtgagtcgcgtgaccgtcagagtcacactcacaaacatttttggacatgaattgaaaattattataattaccagattttttatctatcgtcgaacttgggactgagaaagtagcagcttcgtttgcttcctctgatatttttctacactctctgtctctctctaaagatgaattgtcttcagaatcttcccagcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcaattttatatagtattatttttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggactgggaagattctgaagacaattcatctttagagagagacagagagtgtagaaaaatatcagaggaagcaaacgaagctgctactttctcagtcccaagttcgacgatagataaaaaatctggtaattataataattttcaattcatgtccaaaaatgtttgtgagtgtgactctgacggtcacgcgactcacgacgatacaattgggaccccacggtctgcaccgggaatgcaatcacttttgtttgttacgtaaagtggaacacattactagaaaaagtctccaactttccacaataacaaaaacaccctcttcacaaaactaatatctgctgccacatcagcaccgtcaatattgaattgggttattctgtctaattatttttgttagaggttacttcctcagcgcatgc

SEQ ID NO: 3 - DNA, 4376 bp, Arabidopsis thaliana
atgagtaactctgtagtgcataacttacttaaccggggtttgattcgtcctcttaactttgaacatcaaaacaagctcaactcctctgtgtaccaaacttcaacagcaaatccggctcttggcaagattggcagatcaaaactttacgggaaaggtcttaagcaggcaggacgcagtctggtcactgaaacaggaggaagacctctctcatttgttccacgagctgtccttgccatggatcctcaggcagccgagaaatttagtcttgacggaaatatcgatttactggttgaagtcacttctacaactgtaagagaagtaaatatccagatagcttatacaagtgacacattgttcctacactggggtgcaattcttgacaacaaagaaaattgggttctaccttctcgctctccggatagaactcaaaacttcaagaacagtgcgcttagaactccatttgtgaaatccggtggcaattctcaccttaaactagagatagatgatcctgccatacacgctattgagttccttatatttgacgaaagtcggaacaaatggtataaaaataatggtcagaattttcatataaacttaccaacggaaaggaatgtgaaacaaaatgtttctgttcctgaagatcttgtacagatccaagcatatcttagatgggaacgtaagggtaaacaaatgtacaaccctgagaaagagaaggaggagtatgaagccgcccggacggagctacgggaggaaatgatgcgaggtgcttcagtggaagatctcagagcaaagctgttgaagaaagataacagtaatgaatccccaaaatctaatgggacatcatccagtggacgggaggaaaagaaaaaagtttccaagcaaccagagcgtaaaaaaaattataacactgacaagatccagcgcaagggaagggacctgactaagcttatctataagcatgttgctgattttgttgaaccagaatccaaatcctcatctgaaccacggtccttaacaactctggagatatacgccaaagcaaaggaggaacaagaaaccactccagtctttagcaagaaaacattcaagcttgaaggcagtgcgattttggtgtttgttactaaactttccggaaagacgaaaattcatgtggcaactgattttaaagagccggttacccttcactgggctttgtctcaaaagggtggagaatggttggacccaccttcagatatactgccaccaaactctttgccagtacgtggtgctgttgatacaaaactgaccatcacttcaacagatcttcctagtccggttcaaacttttgagctggaaatagaaggtgacagctacaagggcatgccgtttgtactcaatgctggtgaaaggtggattaaaaataatgacagtgacttttatgtggactttgctaaagaagaaaaacatgttcagaaggattatggcgatggaaagggtacagccaagcatttactggacaaaatcgcagatttggagagtgaggcccagaagtctttcatgcatcgattcaacattgcagcagatcttgtggacgaggcaaaaagtgctggtcaactgggctttgcagggatcctagtctggatgaggtttatggctacaagacagcttgtgtggaacaaaaactataatgttaagccaagggagataagcaaagcgcaggatagactgactgaccttctccaggacgtttatgcaagttatccagagtacagagaacttttgcggatgataatgtctactgtaggtcgaggaggtgaaggagatgtcgggcaacgaatccgtgacgaaattctagtcatccagcggaaaaatgactgcaagggtggaattatggaggaatggcatcagaagttgcataacaacactagtccagatgatgttgtcatctgtcaggcattgatggattatatcaaaagtgactttgacttaagtgtttactggaagaccttgaacgataatggcataaccaaagagcgactcttaagttatgatcgtgctatacattctgaaccaaattttagaggagaacaaaaagacggtcttttgcgtgatcttggacactacatgaggactttaaaggctgttcattcaggggcagaccttgagtcggctatacaaaattgcatgggctaccaagatgacggtgaaggtttcatggttggggtgcagataaatcctgtatcaggattgccttctggatatccagacttgcttcgtttcgtcctagaacatgttgaagaaaagaatgtagagccacttcttgagggtttgcttgaagctcgtcaagagctaaggccacttctgctgaagtcccatgaccgcctcaaggatctgttattcttggacctcgctcttgattctactgtcagaacagcgattgaaagaggatatgagcaattgaatgatgctggacctgagaaaatcatgtacttcatcagcctagttcttgaaaatcttgccctctcttcagatgacaatgaagaccttatatactgcttgaagggatggcaatttgccctcgacatgtgcaagagcaaaaaagatcactgggctctgtatgcaaaatctgttcttgacagaagccgactagcactggcaagcaaagctgagaggtaccttgaaattctgcaaccatcggctgaatatcttggatcttgtcttggagtcgatcagtcggctgttagtatatttactgaagagatcattcgagctggatctgcagcagcattgtcgtcacttgttaaccgacttgacccagttcttaggaagactgctaacttgggaagttggcaggttattagtcctgtagaggtcgtcggatatgtcattgttgtggacgaattgctcactgtacagaataaaacctacgatagacctacaattatagttgcaaacagagtgagaggagaggaggaaatccctgatggtgcagttgcggtactgacacctgacatgccggatgtactatctcatgtttctgttcgagcaagaaatggaaagatctgctttgccacatgttttgattctggtatcttatctgacctccaaggaaaagatggaaaactgttgagcctacaaccaacctctgcagatgtagtctataaagaggtaaacgatagtgagctttcgagtccaagttcagacaacctggaagatgcccctccaagtatttctttggtcaagaaacagtttgcgggtagatatgctatatcatctgaggagttcacaagtgacttggttggtgctaaatcaagaaatatcgggtatctgaaaggaaaagttccttcttgggttggtatcccaacttcagttgcgttgccatttggtgtttttgagaaggttatctccgaaaaggcgaatcaggcggtgaacgataaattgctagtattgaagaaaactcttgatgagggagaccaaggtgctctgaaggaaatccggcagacactgttggggctagttgcacccccagaactggttgaagaactgaaaagtactatgaaaagttctgacatgccatggccgggtgatgaaggtgaacagagatgggagcaagcttgggcagccattaaaaaggtctgggcttcgaaatggaacgagagagcatacttcagcacgaggaaagtaaaactggatcatgactatctctgcatggctgttttggtccaagaagtcatcaatgcggattacgcattcgtcattcacacaactaatccatcttctggagattcatcagagatttatgccgaggtggtcaaaggccttggggaaactcttgtaggagcatatcccggtcggtctctgagtttcatctgcaagaaaaacaaccttgattcgcctctggtgttgggctacccaagcaaaccgattgggctgttcataagacgttcaatcatcttcagatctgattccaatggagaagatcttgaaggttatgcaggtgcaggcctctacgacagtgtaccaatggacgaggaagaccaagtcgtgctcgattacacaacagatcctctgatcactgacttgagcttccagaaaaaggttctctcagacattgcacgcgctggagatgccattgagaaactctatggaactgcacaggacattgaaggtgtgatcagagacgggaagctctatgtcgtccagacacgaccacaagtgtgatcaaattctctgaccacttcttaatgtgtacgttacgttttctgtccagtaaactccttatttgctctataagcaaagagtataatacagcataagcatatagtggattacaaaatgtgtagtacaaagacatttggcattaccattaagatataaataaaaaaactgtttattgg

SEQ ID NO: 4 - DNA, 4136 bp, Arabidopsis thaliana
cacaatctcttcttctgtgtatgtattctttttttctccgatattcgtcgatctctcaaaaatatcctcaaagccaaaaaaaagcaagagtagagataaccaaccaacaaactcataggaagacatatttactttcagggtttagctgtttgattcagcttcgatttagtgtacagtgtgttgattagtataaaaaggatttaaaagaatctgggagatgtgtatagtgattgttcttgctggcgaatgttaacactacgtcgtattgattctcgattgatagtagaagaaaggtgttagttagattgttcgttcatctagttggggtttagtttcggttcctggatggctagttcatcttcatctgagagatggatcgatggtcttcagttctcttccttgttatggcctccgccacgagatcctcaacaacataaggatcaagtcgttgcttatgttgaatattttggtcaatttacatcagagcaattcccagatgacattgctgagttggtccggcatcagtatccatcaactgagaagcgacttttagacgatgtgctggcgatgtttgtccttcatcatccggagcatggtcatgcagtcattcttccaatcatttcatgtcttattgatggctcgttggtgtacagcaaggaagctcatccgtttgcctctttcatatctttagtttgcccaagtagtgagaatgactattcggagcaatgggctttggcatgtggagaaatccttcgcattttgactcattacaaccgtcccatttataaaactgagcagcaaaatggagatacagagagaaattgtctgagcaaagctacaactagtggttctccgacttcagagcctaaggctggatcaccaacacagcatgaaaggaaacctttaaggcctttgtctccatggatcagtgatatactacttgctgctcctcttggtataagaagtgactatttccgatggtgtagtggtgtaatgggtaaatatgctgctggagagctcaagccgccaaccattgcttctcgaggatctggtaaacatcctcaactgatgccttcaaccccaagatgggctgttgctaatggagctggtgtcatactgagtgtttgtgatgatgaagttgctcgatatgagactgctacgctgacagcggtcgctgtccctgcacttcttcttcctccgccaacgacatccttagatgagcatctagttgctggccttccagctcttgaaccatatgcacgtttgtttcatagatactatgccattgcaactccaagtgctacgcagagacttcttcttggactcttagaagcaccaccgtcgtgggctccagatgcacttgatgctgctgtacagcttgtggaactccttcgagctgctgaagattatgcatctggtgtaaggctacccaggaactggatgcatttgcacttcttgcgggctataggaattgctatgtctatgagggcaggtgttgctgctgatgctgcagccgctttgcttttccgcatactctcacagccggcactgctttttcctccgctaagtcaagttgagggagtagaaattcagcacgcgcctattggtggctacagttcaaattacagaaaacagatagaagttcctgcagcagaagcaaccattgaagccactgcccaaggaattgcctcaatgctttgtgctcatggtcctgaagttgagtggagaatttgcactatatgggaagctgcttatggtttgatccctttaaattcttcggcggttgatcttcccgaaatcatagttgctaccccactgcaacctcctatcttgtcatggaatttatacattccactcctcaaagtacttgaatatcttccacgggggagtccttcggaagcatgcttgatgaaaatatttgttgccactgtggaaacaatactcagtagaacttttccgcctgaatcttccagggaactaaccagaaaagctagatcgagttttaccacaagatcagcgaccaaaaatcttgctatgtctgagcttcgtgctatggtccatgctctctttttagaatcatgcgctggtgtggaattagcttcacgcctactttttgttgtgttgactgtatgtgttagccatgaagcacagtctagtggtagcaagagaccgagaagtgaatatgctagtactactgaaaatattgaggcgaatcaacctgtatctaacaatcaaactgctaaccgtaaaagtaggaatgtcaagggacagggacctgtggcagcatttgattcatacgttcttgctgctgtttgtgctcttgcctgtgaggttcagctgtatcctatgatctctggtggggggaacttttccaattctgccgtggctggaactattacaaagcctgtaaagataaatgggtcatctaaagagtatggagctgggattgactcggcaattagtcatacgcgccgaattttggcaatcctagaggcactcttttcattaaaaccatcttctgtggggactccatggagttacagttctagtgagatagttgctgcggccatggttgcagctcatatttccgaactgttcagacgttcaaaggccttgacgcatgcattgtctgggttgatgagatgtaagtgggataaggaaattcataaaagagcatcatcattatataacctcatagatgttcacagcaaagttgttgcctccattgttgacaaagctgaacccttggaagcctaccttaagaatacaccggttcagaaggattctgtgacctgtttaaactggaaacaagagaacacatgtgcaagcaccacatgctttgatacagcggtgacatccgcctcaaggactgaaatgaatccaagaggaaaccataagtatgctagacattcagatgaaggctcagggagaccctcagagaagggtatcaaagatttcctcttggatgcttctgatcttgcgaatttcctcacagctgatagactcgcagggttctattgtggtacacaaaagcttttgaggtcagtgcttgcagagaaaccggagctgtctttctccgttgtttcactgttatggcacaaactgattgctgctcctgaaatccagcccaccgcagaaagcacctctgcgcaacaaggatggagacaggttgttgatgcgctatgcaatgtcgtatctgcaacgccagcgaaagcagcagcagcagttgtccttcaggctgaaagggagttgcagccttggatcgccaaagatgatgaagaaggccaaaaaatgtggaaaatcaaccaacggatagtcaaagtgttggtggaactcatgcgcaatcatgacaggcctgagtcactggtgattctcgcaagtgcatcagatcttcttctgcgggcaactgatggaatgcttgttgatggagaagcttgtacattacctcaacttgagctacttgaagccacggcaagagcaatacagccggtgctagcttgggggccatctggactagcagtggtcgacggtttatccaatctattgaagtgtcgtctaccagcaacaatacggtgcctttcacacccaagtgcacacgtacgtgccttaagcacgtcagtactacgtgatatcatgaaccaaagctccatacccatcaaagtaactccaaaactgccaacaacagagaagaacggaatgaatagtccgtcctatcgattcttcaacgccgcctcaatagactggaaagccgatatccaaaactgtttaaactgggaagctcacagcttgctctccacaactatgcctactcagtttctcgacactgcggctcgggaactcggctgtactatatccttgtcccaataacgagcacccacttttgtttttggtaaattttagttctctagacaaaacatttggacgtagaccaagaagaatatatatatagtttgttgtatgtaatgttgtaatgatgagtgactgacgcaatcactcccaccggcgttggatttgctctcgctcggtgtcttatataactcaacctcttctctgtacattttaaatgacgaagtagctcaatctttttttttgtgcgtctggtgtttagtcttcagtggattctaaatcgtaatgta

SEQ ID NO: 5 - DNA, 4150 bp, Arabidopsis thaliana
atgctgtacctgtgtaattgatgctgtttacaaaaagttttttctttgatgcaggaaactacttgtgtgtattttgaagagcttctatagatttgatacgtttaacgcataaaaacagctcaagtttgtgaagtggaacaagctgccatggcaacctctaaatcccaacaattccagctaattgaagggatggagcttcagatcaccgtcactggattgccgaatgggagcagtgtaagagctgagtttcacctgaagaactgcactcgcgcatggattcttcattggggttgtatttaccaaggaaataaccattggtatatcccatctgaacattcctcgaagcaaggtgcattgcagactacctttgtgaagagtggggatgcatatgtagtgatccttgagttgcgggatccaagggtacgcgcaattgaatttgttttgaaagatggtagccataacagatggttgagacagcataatggaaacttccgtgttgagattccctggaatgatctgcatgctcatcatcggataccgaagactctgatagaaagaagagcacataagatatgggaccggaagggacgaccacaaagctctgcacgtgaacaacagatagactatgacaatgcagtaagagagctccacgccgagctcgctagaggaatatctttagacgaacttcaggctaactctacagtaccagtggagaaagaagaaaccagtgagccacatcacacaatgatccaatcatatcgccggaagcatgatgttcagaaatggttacagaaatatacggaaccaatcaacagaagtggaagtgtgaaaagttcagcccttgcagaactctccaagagatctgtgggccaagaaaatctagtttcacagaaaagctttcatgtcagaaactacgagatcacggtcctccaaagggatgttaagggagattgtcgcttatggattgccacgaacatggcaggtccaacagttctccattggggagtcgcaaagtcatctgcaggagagtggttgataccaccacctgacgtgttacctgagaaatcaaaatttgttcacggagcatgtcaaacacaatttaccgatatgtcgagcagagaacactcttatcagtttattgatataaatttaaaacgaggtggttttgttggtatccaatttgtaatatggtcgggaggctactgggtgaacaataacggagccaacttcgttgtaaacctgaagtcagcagacagtaccagtggtaagcttgacgtggatgaaaagtatgttctcaaatggttacttgatgaaatatctgagcgagagaaagaggctgagagatcattgatgcacaggttcaacattgcaacagagttgaccgagcgttgtaaggatgaaggagaaggtggatgtattggtataatggtgtggatgagattcatggccaccagacatctcacctggaataagaactataacgtgaaacctcgggaaattagtgaagcactagaaagattcaccaatttgatggagaaaatatatttgcagcaaccaaataagagagaaattgtgagactaactatggcacttgtgggtcgtggaggtcaaggtgatgttgggcagagaatccgtgacgaaatccttgttattcagagaaataaccactgcaaatccggcatgatggaagagtggcaccaaaagttgcacaacaacagtagcgcagatgatgtgataatttgtgaggctctcttgaactatgtgagatctgacttcaggatcgatgcatactggcaaacactacagaccaatggtctcacaaaagaaaggcttgcaagttatgaccgccccatagtatcagagcctcgtttcagaagtgattctaaagaaggactcatccgtgaccttacaatgtacttgaaaacattaaaggcagttcattcaggtgcagaccttgagtctgctattgatacgttcctttctccatctaagggtcatcatgtctttgctgtcaatggtttatcaccaaaattgcaggatctactgaatttagttaagaggcttgttcgcgaagagaacactgagcccctaatagagaagttagttgatgctcgcattcagttacaccctgcactacgagcacctcgcacgagggcaaaagatttactatttttggacattgcgttggagtcatgttttaaaacaacaatagagaagagactcatctctttaaacttcaataacccaccggaaattatatatgtcatctgcgtggtgcttgagaatctgtgcttatctatagttaacaatgaagaaatcatattctgtacaaaggattggtaccgtgtcagcgaggcttacagacctcatgatgttcagtgggcattgcaaacaaaagcggtccttgaccgtttacaactagtacttgctgatagatgtcagcattattttacaataatacagcctactgcaaaatatcttggtcaactgttacgtgttgacaagcatgggattgatgttttcactgaagaggttataagagcagggccaggagccgttttatcaactcttgtaaacagatttgatccttctctaaggaaaatcgcgaatttaggctgttggcaggttatcagctcggctgatgcatatgggtttgtggtttgtgtgaatgagttaattgttgttcagaacaaattctactcgaagccaactgtaattattgcaagtaaagtcacaggagaagaggagatccctgctggtgttgtagccgtgctgactcctagtatgattgatgtcttgtctcatgtatctattagagcaagaaacagcaagatatgctttgctacatgcttcgatcagaatgttctcagcaatctgaagtcgaaggaagggagagcaatatctattcatacaaagtctactggtttagttatcagtgatggcaacaactctgatgtctctgttcgtcatatttttatttcctctgttccacggggagtgatctctaaggggaagaagttttgtggccactatgtgatttcatctaaggaattcactgatgaaagggttggctcaaaatcatacaatataaaatttctacgtgaaagagttccatcatggatcaagatacctacctcagctgctcttccattcggaacatttgagaatatactctcagatgattccaataaggatgtagcacgcaggatttctgtccttaaagattctcttaacagaggagacctgacaaaacttaagtccattcaagaagctatcttacaaatgagtgctccaatggctctgagaaatgaactgatcacaaaattgagaagtgaaagaatgccttatcttggtgatgaatcaggctggaaccgctcctgggtggcaattaagaaggtctgggcttcaaagtggaacgagagagcttatgtcagttgcaaaaaaaataagcttgatcatgatgcggtctgcatggctgtgctgattcaagaagtcatttgtggcgattatgctttcgtcattcacacaaacaatccggtctctggtgactcttcagaaatatacacagagattgtgaagggtttgggagagaccttggttggagcatatccaggacgagcaatgagcttcatcaccaagaaaacaaacctcaagtctccaaccgtgatcagttacccaagtaagaggataggtctgtattctaaaccctcaatcatattcagatcagattcaaacaacgaggatctcgaaggcaacgcaggcgctggactttacgacagtgtgataatggatgaagcagaggaagtagtggtggattactcaagggagccactaataatggacaaatcctttcgagtgcgtctattctcagcgattgcagaagctggaaatgtgatagagtcaatctatggttgtcctcaagacattgaaggtgttgtcaaaggtggacatatctacatcgtccaagctagaccccaagtttaagctctctttttctacttgtaccattccctaagttttcaatcaaatcttataattacaagctaatcttttatatgtttgctattacacatgtaatatttctacatcatggcacactcctgatacaaacaaacatgattcaaagtttcataataaatgattgtgtgat

SEQ ID NO: 6 - DNA, 1509 bp, Arabidopsis thaliana
acaaacatttttggacatgaattgaaaattattataattaccagattttttatctatcgtcgaacttgggactgagaaagtagcagcttcgtttgcttcctctgatatttttctacactctctgtctctctctaaagatgaattgtcttcagaatcttcccagatgttcagtctcacctctgctgggattcgggtgcattcaaagagatcattcttcttcttcttcttctttgaagatgctaatatcgcctccgatcaaagccaatgatccaaaatctcgacttgttttacatgcagtatcagagtcaaaatccagctcagagatgagtggtgttgcaaaggatgaagagaaatctgatgaatatagccaagacatgactcaagctatgggtgctgttctaacttacaggcacgagttaggaatgaactacaactttattcgtccagatctaattgttggatcctgcttacagacccctgaagatgttgacaagcttcgtaaaattggagttaaaaccatattttgcttgcaacaagatccagacctggaatattttggagtagacataagcagcatccaagcctatgctaagaaatatagtgatattcagcatattcgctgtgaaattagagactttgatgcatttgatttgagaatgcgtcttccagccgtggttggtactctttacaaagctgttaagcgaaatggaggagttacatatgtgcactgcactgctggaatgggaagggctcctgctgttgcgttgacatacatgttctgggtgcaaggctataagcttatggaagctcataaattacttatgagcaaaaggtcgtgctttccgaagctggatgctatcagaaatgcaacaattgatattcttacaggactcaagaggaagactgttactctgacactgaaagataaggggttctccagagtagaaatttctggccttgacattggatggggacagaggatacctctaacactggacaagggaacaggattctggatcctaaagagagaactgcctgaaggacagtttgaatataaatacatcatagatggtgaatggacacacaatgaggccgaaccgtttataggacctaacaaagacggccataccaacaattacgctaaagtagtggacgatccaacaagtgtggatggtacaactcgggagagactatcgagcgaagaccctgagctgttggaggaagaacgctcgaaactaatccagttcttggagacttgttctgaggcagaagtttgagagttgagagcatccgactagactatgtgaatgtacaacttactagtaacaataaaatgcttcatgttagtaatacatacagttacgcttatagcagcaaaagaaaaaaacatgaagttagtagtactatgttcaagtataatatatagtttaaaatctttgttttttcttaattacattctttcaccatgtgaagattggtcaaaattgatgaaaatgtaatctttggttg

SEQ ID NO: 7 - DNA, 2012 bp, Arabidopsis thaliana
acagttccaacttcttccatacacacacattctcggaaaaatctctattggatcggtttaatggcgtttcttcaacaaatctccggtttaggagctctcgaacgatcttgtccgtcaattatgatcggatcttcattccggagcggtaatggccgtgtattcgacggaagaggaattgcgtacttgggttcgagggaaaagttcggattcaacagacgaagaagagtcgttcttagagttgttgcgatgtcgtcttcttctactccgttcaaaatgaatttgaatgagtatatggtcactctcgagaagcccctcgggattcgcttcgcactttcagcagatggcaagatcttcgtccacgccatcaaaaaagggagcaatgcggagaaggcgagaatcatcatggtaggagatacactcaagaaggctagtgattcttctggtggtactcttgtggaaatcaaggattttggtgataccaagaagatgctggtagagaaaactggatcctttagccttgttcttgaaagaccattctctccctttcccattcagtatctgcttcatttgagcgaccttgatttgctctataacagagggcgtgtgtcttttgttacttggaacaaaaacctcttatcatcgaatctcagagcatcatcacaaggcagtggtaactctggctatgctgcgttttcctccaaattctttactcctcagggatggaaacttttgaatagacagagcaattcctttcagtcagggactaagaagaacattctttctccacctatcagtccactggtctctgtcttttcagaagatgttcctggagatggggaatggggttatgggaatttccctttagaggaatacatcaaagcccttgaccgttctaaaggagagctttcttataaccatgctcttggaatgcgctacagtaagattacagagcaaatatatgttggctcttgcatacaaacagaagaggatgtggaaaacttgtcggaagctggaattactgccatactaaatttccaaggtggaactgaagctcaaaactgggggatcgattcacaaagtatcaatgatgcatgccagaaatctgaagtcttaatgatcaactaccctataaaggatgcagattcctttgatcttaggaagaagctaccactttgtgttgggctcttactacggttactgaaaaagaaccatcgtgtctttgtaacttgtaccactggttttgaccggtcatccgcttgtgtgattgcttatctgcactggatgactgatacctctcttcatgctgcttatagtttcgttactggattgcacgcttgcaaacctgacagacctgcaattgcttgggcaacatgggatcttatcgctatggtggacgatggcaaacatgatgggactccaacacactctgtaacctttgtgtggaatggacatgagggggaggaggtattgttggttggggatttcacaggaaattggaaagaaccgataaaagcaacgcacaaaggcggtccacgttttgaaactgaagttagacttacacagggaaaatactactacaagtacataatcaacggtgattggaggcattcagcaacttcaccaacggaaagagatgaccgaggaaacacgaacaatataattgtagttggtgatgttgctaacgtcaggcctacaattcaacaaccaagaaaggatgcaaatatcataaaggtgatcgagagagtgttaacagagagcgaaagatttaggctggcgaaagcagctcggtgtatcgcattctcagtttgtccaataagactatgcccaaagtcgtagaagttttgttttggcataagttaaaatgttacatccattcttctgtctctatccattcctcgaggtacttaaccctaatatcgttgtgaacaacattgccacattcgcgcactctatgacaagagaatgttgacattatgttgtgtgtttgcatgaaaaaatgtcgacatagtttc

SEQ ID NO: 8 - DNA, 2131 bp, Arabidopsis thaliana
atgcttcccctataaattatcttctcttcctccttctccgatctctcctccgtcacacaaaacttccgatttcaaaaaaacaatttgaaatttgtcaaaaggaatattctgacgatcgattcttgattcgttttctcgcgtatagagagaaaaatggcgcttaatttatcgcatcagctaggagtactcgccggaactccgatcaaatccggtgaaatgacagattcatcgttgttatcaatttcaccaccatcggcgagaatgatgacacctaaagcaatgaatcgaaactacaaggctcacggaacagatccatcaccgccgatgagtccaattttaggtgcgacacgtgcggatctgtcggtggcgtgtaaagctttcgcggtggagaatggaatcgggaccattgaagaacagagaacgtatagagaaggagggattggtggaaagaaagaaggaggaggaggtgttcctgtgtttgtgatgatgccgcttgatagtgttaccatggggaacacggtgaatcgaagaaaggcgatgaaagcgagtttgcaagcgttgaagagtgctggtgttgaagggattatgattgatgtgtggtggggtttggttgagaaggaatctcctggaacttataattggggaggttataatgaattgcttgagctggctaagaagcttggtcttaaggttcaagctgttatgtcgtttcatcagtgtggtggcaacgttggtgactctgtcactattcctctgcctcagtgggttgttgaagaggttgacaaggatccagaccttgcatacactgatcagtggggaagaaggaaccatgagtatatatcacttggcgctgatacactccctgttctcaaaggtagaacacctgtgcaatgctatgcggatttcatgcgtgcttttagagacaacttcaagcatcttcttggagagaccattgtggaaatccaagtgggaatgggacctgcaggagagctgcgttacccttcgtaccccgagcaagaagggacatggaagttcccagggattggagccttccagtgctatgataagtactcgttaagcagcttgaaagcagcagctgagacttatgggaagccagagtggggcagtactggcccaaccgatgctggtcactacaataactggccagaagacacccagttcttcaaaaaagaaggtggtggttggaactcagagtatggagatttcttcctctcatggtactcccagatgctgttggatcacggcgagcggatcctatcctcagccaaatcaatattcgaaaacatgggtgtcaaaatatcggtcaaaatcgctggaatccactggcactacgggacccgctctcacgcgcctgagctcacagcagggtactacaacacaaggttcagagacgggtacctcccgatcgcccaaatgttagccagacacaatgccatattcaacttcacatgtatcgagatgagggatcacgaacagcctcaagacgcactttgtgcaccagagaagctagttaaccaagttgctctagccactctagccgcagaagttcctctagctggtgaaaacgcattgcctaggtacgacgactatgcacacgagcagattctcaaggcctccgcgctgaaccttgatcaaaacaatgagggagaacctcgagagatgtgtgcatttacttacctaaggatgaatccggagttgtttcaggcggataactggggaaaattcgtagcgtttgtgaagaaaatgggtgaagggagagactcacatagatgtcgggaagaagttgagcgtgaggctgagcattttgtgcacgttactcagccgctggttcaagaagctgcagtggctctcactcactagagagatagattgtggcatggttctatggtttatgtttgtatgcgattataatgaaatggttatatgacaatgccaaaaaagatgtttaggcttgttggagcttgtgtatgtattgttagtaaaactgaaatgtatgccacagctctggtttttttccggtcaggttgtggcatgctactacttttaatatatgctaaaagatgtacatgttctttaatgaagccaaaataaatagcatttctttaataca

SEQ ID NO: 9 - DNA, 2000 bp, Arabidopsis thaliana
caatgattcttggatcccatgaataagccttcacgtggcttttgatttcacattccacgaaaatccaattttaactgaatttgagataattttggtttagtgatggcgattaggttgaatcatagtgtaattccagtgagcgtgaagcttggagctccgacgagagtatcggctcggagctcgttaccgttcagcgttggagattggcgtggtgtttctacattttccggtgctaggcctttggttctcgcgaaggtgaaactgagagcagagagtactgaggaagatcgagttccaattgatgacgacgacgattctactgatcagctcgttgatgaagaaattgtgcatttcgaagagcgtgattttgctggcacagcgtgtgttcctgtatatgttatgctaccactgggagtgattgatatgaactcggaagtggtggaaccggaggagcttttggatcagttaagaactttaaagtcggttaatgttgatggtgttatggttgactgctggtggggaattgttgagtcacatacaccacaggtttataactggagtggttacaagaaactttttcagatgattcgtgagcttggacttaagattcaggttgttatgtcgtttcacgaatgtggaggcaatgttggagatgatgtgcatatacaaatccctgagtgggtgagagaaattggtcaaagcaatcctgacatctacttcactgacagtgctggcaggcgtaacactgaatgccttacttggggaattgacaagcaacgtgttttaagaggacggactgctcttgaggtttactttgattacatgagaagctttcgtgtggaatttgatgaattctttgaggagaaaatcatccctgaaattgaagttggactaggtccatgtggggagctgaggtatccttcataccctgcacaatttgggtggaaataccctggtattggtgaattccagtgttatgataaatatttgatgaatagcttaaaggaggctgcagaagtaagaggccacagcttttggggcagaggacctgataacacagagacttataattcaacaccacatgggactggattttttcgcgatggaggcgattatgacagctactatggtagattctttcttaattggtactcaagagttctgattgaccatggtgatcgtgttcttgctatggcaaatttggctttcgaaggaacttgcattgctgcaaagctctcaggtatacactggtggtataaaacagctagtcatgctgctgaactcactgctggcttctacaactcctcaaaccgtgatggctatggtccaatagctgcaatgtttaagaaacatgatgctgctctcaacttcacatgtgtggagttacggacacttgatcaacatgaggatttcccagaggctctggcagatccagaaggtctagtttggcaggtgctgaatgctgcttgggatgctagtatacctgttgctagtgaaaatgctcttccttgttatgacagagaaggttacaacaagatacttgaaaacgcaaagccccttaccgatcctgatggtcgccacctttcatgtttcacttacctgaggcttaacccaactctgatggagtctcaaaacttcaaagagtttgagagattcctcaaacgaatgcatggtgaagccgttccggatcttggtttggctccagggacacaagagaccaaccccgagtgaatatatatagtcctccaccatgctactaaaaacacttattagcgcctggttacatataataagaacgtcttggcttgggtttaccaattcagcatagctaagttggagggaatcaaaaccaagattgtaaactgagcttgtaagttgtaagatgaatagtgtatagaacaatataagtggtatgtccattgatatttcagaagaaattgctagcaacattacaggctgtgttacacacaaaaagagctataacaacaaagaaattctta

SEQ ID NO: 10 - DNA, 2035 bp, Arabidopsis thaliana
aaacacaaacatatcttctatcaaacaccaacagctctattctctacctcatttctcatcataacaaagagagagaaaaaaactatggaattgacactgaattcctcgagttctcttatcaaacgtaaagatgccaagagttctagaaaccaagaaagttcctccaacaacatgacctttgcgaagatgaagccgccaacatatcaattccaagcaaagaactcggttaaggaaatgaagttcactcacgagaagaccttcacgccagaaggtgaaacccttgagaaatgggagaagctccacgttctctcatacccacactccaagaacgacgctagcgttccggtgttcgtcatgttaccgctcgacacagtaacaatgtcagggcatttgaacaaaccacgagccatgaacgctagtttgatggccctgaaaggagctggtgtggaaggtgtgatggtggatgcttggtggggattggtggagaaagatggacctatgaattataactgggaaggctatgccgagcttatacagatggttcaaaagcacggtctcaaactccaggtcgttatgtcattccatcaatgtggaggaaacgtaggagactcttgcagtatccccttgcctccatgggtgcttgaagagatcagcaagaaccctgatcttgtctacacagacaaatctgggagaaggaaccctgaatatatctccttgggatgtgattctgtgcctgtcctaagaggaagaacacctatccaggtctactcagatttcatgaggagcttccgtgaacgatttgaaggctacataggaggagttattgcggaaattcaagtaggaatgggaccttgtggagaattgagatacccatcataccctgagagcaacgggacctggagattccccggaattggagagttccagtgctacgacaagtatatgaaatcgtcacttcaagcatatgctgagtcaatcgggaaaactaactggggaacaagtggacctcatgatgccggcgagtacaagaacctcccagaagatactgaatttttcaggagagacggaacatggaatagcgagtatggaaagtttttcatggaatggtactccgggaagctgctagaacatggagaccaactcctatcttcagcgaaaggtatctttcaaggaagcggagcaaagctatcaggaaaggtagctggaattcactggcactacaacaccaggtcacacgcagctgagctaaccgctggatactacaacacaagaaaccatgacgggtatctgccaatagctaagatgttcaacaaacatggagttgtgctcaacttcacctgcatggagatgaaagacggggagcaacctgagcacgcgaattgctcaccagaaggtctggtcaagcaagtacagaacgcgacaaggcaggccggaaccgaactagcaggggagaacgcgctagaacgatatgactcaagcgcattcggacaagtggtagcaacaaataggtcagattctggaaatgggttaaccgcatttacttacctaagaatgaacaagcggttatttgagggtcaaaattggcagcagttagtggagtttgttaagaacatgaaggaaggtggtcatgggaggagactctcaaaagaagacacaactggaagtgacctttatgttggatttgtcaaaggcaagatcgctgagaatgtggaggaggctgctttagtgtaatttcccacataggtacatacatatagtgtggtgtttattgtattcctgtctgataaataactagagagatcaaaccagtaagagtgttaaagctatagatttgcacaattctgggtcagagtcagagcaaagagaagcaaaatcaagatgatgtacacttagatgtttcctatgagttttccttgtacatcatcttcatactcttaatctcaaatactatgcatttttctcctacagttttatattatctcaatcaatgtgagtttctcttatcatcaatcaatatgcatctctcgttcaaacg

SEQ ID NO: 11 - DNA, 1982 bp, Arabidopsis thaliana
ggtcgttaaaaagaaaagaaaaagagaaatagaattttgcgtttcaagtactccatctccgtctcttttcctccgcatctaaaaccatctgagaaatatctccgacgaaggagaagaaacgaaaatgacggagactggagtaatcggatgtggctgtcgcggagtcaccggcggcaatttcttccatcccggaggattttctttgaaatcttgtttcctcgagcagagtacaaagcgtaatcgtaactttttccgaagcgtttctatgattcctcctttcaaacgcggtcgtttcatcactaagttgcgttccgtcgccgggaacagccgaatctttagcatggatgctcgagagaaatcacgatcgtttgtgttggtatcatcaaggcacaagagagttccagtttttgtgatgatgccgattgatacatttggaattgatgcttctgggtgtccaaagattaaaaggctcaaggctttaactgtatctcttaaggcactcaagttagctggtgttcatggaatcgcagttgaggtttggtgggggattgtagagcgtttctctcctcttgagtttaaatggtcactgtatgaagagctttttagactgatttctgaggcagggttgaagttacatgttgctctttgctttcattcaaatatgcatttgtttggtgggaaaggaggcatcagtcttccactctggatccgagagattggagacgtcaataaggacatatactatagagataaaagcggattttccaacaatgactatctcacacttggagtcgatcaacttcctttgttcggtggccgtactgctgttcaatgctatgaagattttatgctcagtttttcaacaaaatttgagccatatcttgggaatgtgattgaagaaataagtataggtcttggtccttcgggggagcttagatatcctgcacatccttctggagatgggaggtggaaatttcctggaattggtgaattccaatgccatgacaagtacatgatggaagacttgatggcagtggcatcccaagaaggcaaacctcaatggggaagcagagatcctccgaataccggctgctataatagctttccatctggagttccgttctttgaggagggcaatgatagctttctctctgactatggtcgtttctttctagaatggtacagtgggaagttgatttgtcatgctgatgctattcttgcaaaggcagccgatgtcttgcggagacgtcaggaagaagagaaaagctctgtaatgctggttgcaaaaattggtggaatctattggtggtataagacatcttcacaccccgctgaactaactgcaggttattacaacacctccctcagggatggttatgatcctgtagcttccgtcttgtctcgtcatggtgctgctctcaacatcccctgcttggatatggcagatagtgaaatacctgagaaatatctttgcagccctgaaggattacgtagacagatacatgatgtttcgaagaagtggacaatacatgtgactggtagaaacacaagcgaaagatttgatgagatgggactaaggcaaatacgagagaactgtgtgcaaccgaatggcgacactctaagatcatttacgttttgcagaatgaatgagaagatctttagggtcgagaactggaacaactttgtccctttcattagacagatgagtgcagatatgtaaacacactcaccacccattggtgtgtttgtcttttgttacagaatcgatctttcctcaccatattgctttttcagatatgtgatgaggagaactaatcaaatcaaaaattgttatatgataaaatctctctctgtttctattcttcattttgttctatctctattgcaaaagtcttgcagaaacctttttcattggatcaaaaagttacattgtatgttttgattatcagaaagcgatccaatacagaaggtttaatcacagc

SEQ ID NO: 12 - DNA, 1853 bp, Arabidopsis thaliana
agttccgtcaagtgcaaggtaccatgacaagcgtattaggaatgatgaaccctaatctgatcaacggaagaaatctccacaaaggatcttcaatttttgtacaagataaagaaactaaaaaacgagtccaatggagattatcaatcaaggagggatctctaagaacgcatcaagccactgcttcttctgcaacagaaccaaaggcgacagagttcaatacgacgacgtatgaagacaaaatgttgaccaactatgttcctgtctacgtcatgcttcagcttggagtcatcacgaacgataacgttcttgaaaacgaagagagtctcaagaaacagctcaagaaactgaaacaaagtcaagtagatggagtgatggttgacgtatggtggggaatcgttgaatcaaaaggtccaaagcaatatcaatggagtgcttatagaaacttgtttgcgatcgtacaaagctttggactaaagttacaagctataatgtcgtttcatagatgtggaggcaacataggagacgatgtcaacatcccgataccgaaatgggtactcgaaatcggggattcgaatccggatatcttctatacgaacaagagtggtaatagaaacaaagagtgtttgtctctctctgtagataacttgtctctcttcagaggacgaacagctgttgagatgtatagagactacatgaagagctttagagagaacatggaagatttcataagctctggagttataatagacattgaagtagggcttggtcctgctggagagcttagatatccttcttacagtgagactcaaggatgggtttttccaggaattggggaatttcaatgttatgacaagtatctaagatcagactatgaagaagaagttagaagaatcgggcatccagagtggaaacttccggaaaacgcaggcgaatacaacagtgttccaggagaaacagagtttttcgagtactcgaatgggacttaccttaaggaagaaggaaatttcttcttgtcatggtactccaaaaagttacttctccatggtgatcagattcttgatgaagctaataaagtatttctcgggtgtaaactcaagatagcagcaaaagtctctggaatccattggtggtacaaaactgaaagtcatgctgcagaacttacggcgggttattacaatctcaaaaacagagatggttatcgtgcgattgcgaaaataatgcgtagacatcacgctatcttgaatttcacttgtctagagatgaaaaacacagagcaaccagcaaaagctaagagtggacctcaagaacttgttcaacaagttttgagctctggatggagagaagggattgaagttgcgggcgaaaacgcgcttccaagattcgatagaaatggatataaccagatcatactaaacgcaaggcctaatggagttaaccaagatggtaaaccgaggatgtttgggttcacatatctcagactgtctgataaacttctcaatgaaccaaatttctcaacgtttaaaatgttcttgaaacgaatgcatgcgaaccaagagtattgttcagagcctgaaaggtataatcacgagctgcttccgttggaaagatcgagaaacgatgaatccttggagatgtttatggaagaaacagaaccatttgatccatttccatggctggacgagacagacatgagtattagaccctttgagagtgtactgtctctattgcgaagcacatttttgagaaagaagtcctagagaatatgctaaggggaaattctataaacgtataagatttctgaagtaaatatgtaaagatttgagataaatatataaaagtaaataattcatcct

SEQ ID NO: 13 - DNA, 2855 bp, Arabidopsis thaliana
gccaagccttagcttcacacactcctctttctctcagacacgctcgagcgctacacacgcgcgaaagctcgtcttccaattcacgctgcacgctccttcacgcgcttcctccatctcacggccagatccgcctcttttcacccgccggagatccaccgtcttcttctcctctctcttggtttcgctgacggcgacagagggggaaacctttgatcggaagctccatttatagggttttttagtattgctcgatgattcgcccagagggccgagaattctgaccaatttcgagaggaggcttgtgattgttgaaagaattgtgcttagtgtctcttactcacagaggtcaatgatatatggttaggatctttgatgaagttaaactgagagcagcttatgcacattgagagattgaagattaatttcgttgatactaaaattatctccaattttctaactaactgtaagcaaggaaatatcatagaatatttggctgcattgtccagaaggattacacgtggagtgttaaaaccagagaagtaaagatggaacatttcatcataagagggcaataacgttcttaaactaacagaggtttgggtggttgggttataagagaagtaatggcgacggatatgcataaactgcttggaactagtgaagaggatgatgacgaagaaatggacatggatgtgaaggaagaagatgatggagaccgacgaaatagagacaaacacgcagcttcaggcagtagtagtaatgacgagtttatgtttcagcagtcaatgcaagatcaagtgggcactcctgggggaggaggaagtcgcagaagtagaccgcttgaagagaaggagcgaactaagttgagagagcgccatcgaagggctataactgcaaggatactaggcgggttacgaaggcatgggaattataacctgagagttagagctgatattaacgatgttatcgcggctttggcaagggaagctggatgggttgttcttcctgatggaactacttttccatccaaatctcaggggacaaagcctactggtggttcttctgcggttgcggcaggttcatcagcttctcacattgcatcacagcaaacctcacctcctgctcttagagtggtatcatctgggcttagaagcccagtggaactcagctcttgccgtatgaaaggtgttttcacacccgctccatcaccgtatgatatgctcccaatccaatctccagaactggttggcagtgtaaataaggctgaggggcttgttggttgttcagtcgatgttatcaactctaaacaaattctcgagattcctccaaatctgacggagcaagatttctctggcactccttatgttccggtttatgtgatgttaccacttggggttatcaatatgaagtgtgaattggctgatcgagatggactgctaaagcacttacgaatattgaaatcaatccatgttgatggggttaaagtggattgttggtggggaatagttgagggtcattccccccaggagtataattggactggttacaggcagctttttcagatggtccgcgatcttaatcttaagatacaggttctaatgtcctttcatgaatgtggaggcaatgttggtgatgatgtttgcatcccacttcctcattgggtggcagaaataggacgaaccaatccggacatatatttcactgatagagagggaagacgaaacccagaatgcctttcttggggtattgacaaggaacgtattttaagaggcagaactgcacttgaggtgtactttgattacatgagaagttttcggatagaacttgcagagtttctagaggatggtgtaatatctatggttgaaattggactaggcccctgtggagagttacggtacccttcttgtcccataaaacacgggtggcgatatccaggtgttggagagtttcagtgttatgacaagtatctctcgaagagcctgagaaaggcagcagaatcaagggggcacctgttttgggctcgaggcccagataacaccggctcctacaattctcagccgcagggaaccggtttcttctgtgatggaggtgactacgatggcctctatggaagattctttctcaaatggtactcacaggttttgatagaccatgctgaccaaatcctctgtctcgctaaactagtcttcgacagtagctgcattgcagcaaagctcccagacgtccactggtggtacagaacagcaagccatgccgctgagctaaccgctggattctacaacccgagcaaccgagatggttattctgcaattgcatctacacttaagaagcatggtgccactctgagttttgtatccggagaagtgcaagttctaaaccgtccagatgatttctcaggagcattaggagaacctgaggcagtagcttggcaggtattaaacgctgcttgggacagcggtacaccggttgctcgagagaactcgcttgcttgccatgatagagttggttataacaaaatgttagaaagcgttaagtttcggaacgatccagatagaaaacatttgtcatcatttgcctatagtagactcgtcccagctctcatggaaggacataacattgtcgaatttgagcggtttgtcaagaagttgcatggggaagctgtaatgaatcatcatcatcatcatcatcaacaggtttagagttttgttttgtccctggatctgctgaaattatttactgttaaatgttgttaattttagtagcttaccatcatttctgtaaattttgtcattaaaaaattaaggcaactactgtacaagcttacaaagaaaattcaaataatatgggctttatta

SEQ ID NO: 14 - DNA, 2271 bp, Arabidopsis thaliana
gcgagcaacttcaccgcacaaaaatgcatactctcaacaacaccatcaccaccaccaccggttcgcaagatccgaatctcgacccaattccagacccggatcaatttcccaaccggaaccgcaaccaaccacaatctcgccgccctcgcggattcgccgctgcagcagcagcagcgtcgatagctccaacggaaaacgatgtaaacaacggaaacatcgccggaatcggcggtggagaaggaagcagcggcggcggaggaggtggaggagggaaagggaagagagagagggagaaggaaaaagagagaacgaagcttagagaaagacatagacgagcaatcactagtagaatgctcgccggattaagacagtacggtaattttccgttaccggcgcgtgctgatatgaatgacgtaatcgctgctttggctcgtgaagctggttggagtgttgaagccgatggtactacttatcggcaatcacagcaaccaaatcatgtggtacagtttccgacgagatctattgagagtccactttcttctagtacattgaagaactgtgctaaggctgcaattgaatctcagcagcattcagttcttaggaacgatgagaaacttgctcctgtgtctcttgattctattggtattgccgagagtgatcatcccgggaatggaaggtatactagtgttagtcctatcacctccgttggatgcttggaagccaatcagcttatacaagatgttcattccgcagagcaatgtaatgacttcacagagagcttttatgtccctgtgtacgccatgcttccagttggcattatagacaactttggccagttggttgatcctgaaggtgtaagacaagaattaagttacatgaagtcattaaatgtcgatggagtagtaatagattgttggtggggaatagttgaaggatggaaccctcagaaatatgtatggtctggctatagggagctctttaaccttatcagagacttcaaacttaagctacaggtggtgatggcatttcatgagtatggagggaatgcatctgggaatgtgatgatttcgttgccacagtgggtgttgaagattgggaaagacaatccagacatttttttcactgatcgtgaaggaagaagaagtttcgagtgtttgaattggagcattgacaaggaacgggtattgcatggacgaactggtatagaggtctattttgattttatgagaagcttccggtcagagtttgatgacttgtttgtggaaggactaattactgcggttgagattggtcttggagcttctggtgaactaaagtacccatctttcccagaaaggatgggttggatttatcccggtatcggtgagtttcagtgttatgacaaatattcgcaattgagtctgcaaaaggaagcaaagtcacgaggattcactttctggggtaaaggaccagagaatgctggtcaatacagttctcatccacatgaaactgtgttcttccaagaaagaggtgaatatgacagctattatggccgcttcttcttaaattggtactcacaattgctaattggtcatgctgagaatgttctgtctcttgcaaatctcgcatttgaggagacgaagattattgtcaagataccggcaatatactggtcatacaagacagctagtcatgctgctgaattaactgcaggatactacaacccttcaaaccgtgatgggtactctcttgttttcgaaactctgaagaaatattccgtgactgtgaagtttgtgtgccctgggccacaaatgtctcccaatgcgcatgaagaagcattagctgatccagaaggtttaagttggcaggtgatcaacgcagcttgggataagggacttcaaatcggtggagagaatgcgattacttgtttcgacagagatggttgtatgagattaatcgatatagctaaaccaagaaaccatccagatggctatcacttctccttcttcacatatcgtcaaccttctcctctggttcaaggatctacttgctttccagatttggactacttcattaaacgcatgcatggcgacataagagataaacagttttaggatttgcatggtcttaggattcttttagtaagtgtcttttgtttaaaacttcaaaatctgttttgttttgtttattctctcctcaaggcctatatttaagacgattgatcttgtaatctttaaacaccttaacacttcactagttgaagatgaactaactaattaaattcttaaatgc

SEQ ID NO: 15 - DNA, 1926 bp, Arabidopsis thaliana
tatataaaccaaatccataatcctcaccatatccacaatcctcaagctatctcctttgtgtccataatcgaaacgctttttgcgatttatcctaatcttttttctatcttctgctacaaatttggttcttttttggttgaatttggataaaatcataatctcctcttcaattttcttgtgaaatggaagtttcagtgattggaaatcctcaagcgaggatctgcagagcagaattagcttacagagagcttggatttagatttggctctgatgtaatctccggtgaatcgagaaatagggttagtttctgcaaccaaagctctaaatggaaagagatcgcgatacgttgctcttcgagatctgtcaaatgtgaagccatcgtctccgatgacgcttctccgtttctcaaatccactccaaaatctaaatcgctcgagagtgtaaaattatttgttgggcttccgttagacacagtttcagactgcaacaatgtgaatcacttgaaagctattacagctgggcttaaagctttgaagctacttggtgtagaaggtattgagttacctatcttttggggagttgttgagaaagaagctgctgggaaatatgaatggtctgggtacttggcagtagctgagattgttaagaaagtgggacttaagcttcatgcttcactttctttccatggatcgaaacaaacagagataggtcttcctgattgggtggcaaagattggtgatgctgaaccagggatctattttacagatagatatggacaacagtacaaagattgtttgtcgtttgctgttgatgatgttcctgttcttgatgggaagactcctatggaggtttacagaggtttctgtgagagcttcaagtctgctttcgcagattacatgggcaacacaatcacgggaatcacattaggtttgggaccagacggtgagctgaaatatccttctcatcaacataatgccaagctctctggcgcgggagagttccagtgttacgacaaacacatgctttctgctcttaaaggctacgctgaatccactggaaaccctctttggggtctcggtggtcctcacgatgctcctgcttacgatcaacagcctaattcctcttcattcttctcagacggcgggtcatgggaatctcagtacggcgatttcttcttgtcttggtattcgtctcttctcacctcccacgcagaccgagtcctctccgttgcttcatctgcatttagcgggattggagtgcctctatgtgggaagctacctctcttacaccaatggcacaagctaagatctcatccttctgagttaacagctggattctacagctctaatggtcaggacaggtacgaggctatcgcagagatctttgcaaagaactcttgtagaatgataataccaggaatggacctatccgacgagcaccaatcacctgaatctctctcgagccccgagtcattacttggccacatcaaaacttcctgcaagaaacaaggagtcgttgtctcagggcaaaactcatccaccccggttcctggtgggtttgagaggatcgttgagaatctgaaggatgagaatgtaggaattgatctgttcacttaccagagaatgggagcacttttcttctctccagagcatttccatgctttcacagtctttgtccggaacctgagccaattcgagttgtcctcagacgatcaagcctcagaggctgaggttgaggccgagacagctagcataggttcaggcactggtgcacctagtttgcaaaccgcttaatgaaatgcaaaatcataattttttatgtaaataagaaaagtctcctgttgttttagcatttatatagaatgtcatattactgtaaaattatttgcaaccatttcttcacaaaacataaatcctgaagcttaag

SEQ ID NO: 16 - DNA, 2483 bp, Arabidopsis thaliana
ctccgatcgttgaagcaacgaagatatgcttacttcaccttcttcttcttctacatatgatccattctcttccaatttctctccgtcgctcactaatgccttctctagtagcttcaccattcccatgggattgaaattgagtaggagagtcaccagagctcggatcttctctcgtaagattaaggatcgttcaacacttaaggtcacttgtcgtcgcgcacacgaacgtgtggttgaggaagaagcatctacaatgacggagacgaaattgtttaaagtttcttctggtgaggtatctcctcttggtgtgtcacaagtcgataagggaatcaattttgctttgttttctcagaatgctacttctgtcacactctgcttatcactatcccagagtggtaaggatgatacggatgatgatggtatgatagagttggttttggatcccagtgtaaacaagactggagatacttggcacatttgtgttgaggatttgcctctcaataatgttctctatggttaccgtgttgatggtcctggagaatggcaacaagggcatcggtttgatcgtagtattttgcttttggatccctatgctaagctagttaaaggtcatagttcttttggagatagtagtcagaagtttgctcaattttatggaacttatgactttgagagctcgccatttgactggggagatgactacaagtttcctaacatacctgagaaggatcttgttatttatgaaatgaatgttcgtgcttttactgctgatgaatccagtgggatggacccagctataggaggcagttacctcggtttcattgagaagatcccacaccttcaagatctgggtataaatgcagtggaattattgccagtgtttgagtttgatgaactggagcttcagaggcgttctaaccctagggatcacatggttaacacatggggttactcgacagttaacttttttgctccgatgagccgatatgctagtggtgagggagaccctattaaagcttcgaaagagtttaaagaaatggtcaaagccttgcattctgctggtatagaggtcattttggacgtagtttataatcacaccaacgaagcagatgacaaatacccttataccacttcatttcgtggcatagacaacaaggtttattacatgcttgatccaaacaaccaactgcttaacttttctggctgtgggaatacactgaactgtaaccatccagttgttatggaactcatacttgatagcttaaggcactgggtcactgaatatcacgtggatggtttccggtttgatcttgctagtgtgctttgccgagcaacagatggatctccactcagtgctcccccactgataagggcaattgccaaggattctgttctgtcaagatgtaaaataattgcagagccttgggattgtggaggattatatctagtcgggaagtttccaaattgggataggtgggctgagtggaatgggatgtaccgggatgatgttagaagatttatcaagggtgacagtggcatgaaaggaagctttgctactcgggtttctggatcttccgatctttaccaggttaaccagaggaagccttaccatggtgtaaattttgtgatcgcacatgatggattcacattgcgagatcttgtatcgtacaactttaagcacaatgaggccaatggagaaggaggaaatgatggatgtaatgacaatcatagctggaactgtggttttgaaggggaaactggtgatgctcacatcaaatctttacgtactcggcaaatgaagaactttcatttagctttgatgatttctcagggaacaccaatgatgctaatgggagacgaatatggacatacacggtatggtaacaacaatagctacggacatgatacctcccttaacaatttccagtggaaagagctcgacgcaaagaagcagaaccatttcaggtttttctcggaggtgatcaagttccgacattcacaccatgtactcaagcatgaaaatttcctcacccaaggtgagattacatggcatgaagataattgggacaacagtgagagcaagttcctagctttcactctccatgacggtataggtggccgtgacatctatgtggcattcaacgcccacgactactttgtcaaggctctgattccacagccaccaccgggaaaacaatggttccgtgtggctgacacaaacctcgagtcgccggatgattttgtgagggaaggtgtggccggtgtggctgatacctacaatgtggccccattctcttcaatccttctccagtccaagtaagataagcactcacacgataacgcacggtagaggaaaaataaggtttatgagttatgagtaaaaaagatccatgactccatgagtctcttgtaaaagttgattacatgtaaaataataaaatgtacgacatttccaatcacaaataaagatatatttagagcaa

SEQ ID NO: 17 - DNA, 1991 bp, Arabidopsis thaliana
tgattttgtcatctggtggattagagtggagtggagctttgaattggatgatcattatccatgtcgattctacttaggccgtcgtcctctccttcactctgttcttctctcaagcttttccgattatcctctccggattctttaatcgacgcagctgtcctcaggaacaggacaaagccgtcgcagtcgtttcgaatggaggtcgtttcgagtaattccacgtgtctttctagtattagcgtcggtgaagattttccatcagaatatgagcagtggctaccggttccggatccagagagcaggagaagagctggcgttttgctacacccgacgtcgtttcgtggtcctcatggcattggtgatctcggagaagaagccttccggttcatcgattggcttcattctactggttgctccgtttggcaggttcttcctcttgttcctccagacgaaggaggatctccttatgcaggacaggatgcaaattgtgggaacacattgttgatttctctagatgagcttgtgaaagacggcttgttaatcaaggatgagctcccacaaccaattgatgctgactctgtgaactatcagactgccaacaagttaaagagtcccttgattacgaaggcagcaaagaggcttattgatggcaatggtgaactgaagagcaaactgctagatttccgtaacgacccctctatatcatgttggcttgaagatgctgcttattttgcagctattgacaatactttaaatgcatacagttggtttgagtggcctgaaccacttaaaaaccgtcatctttctgccttggaagctatatatgaaagccaaaaggagtttatagacttgttcattgctaagcaatttttgttccaaaggcagtggcagaaagttcgtgagtatgcacggcggcaaggagttgatataatgggagatatgcccatttatgtaggatatcacagtgcagacgtttgggcaaataagaaacatttcttactgaacaagaaaggctttcctcttcttgttagcggtgttcctcctgacttgttcagtgaaactggtcaactgtggggcagccctctttatgactggaaagcaatggagagtgaccaatattcttggtgggttaatcgaataagacgcgcacaggacttgtatgacgaatgcaggattgatcacttcagaggatttgcagggttttgggcggtcccttctgaagcgaaagttgccatggttggacgatggaaggtaggacctggaaagtcattatttgatgccatttccaaaggcgttgggaagatcaaaatcatagctgaagatttgggagttattactaaagatgtagttgagctgaggaaatctatcggagcacctggaatggccgtcctccaatttgcttttggaggaggcgccgataacccacatttacctcacaatcatgaagtaaaccaagttgtatactctggaactcatgacaacgacactattcgaggctggtgggacactctggaccaagaagaaaagtctaaggcaatgaaatacctgtcgatagctggagaagacgatatatcatggtcagtcatccaagctgcattctcttcaaccgctcaaaccgcaatcataccgatgcaagacattctaggacttggaagttctgccaggatgaacactccagccactgaggtggggaattggggttggaggattccaagttcaacgagctttgataatcttgaaactgaatctgacagactcagagatcttttgtcattgtacggacggctttgaggaaaacatttgacgtggaaaagaagaagaatctttggttttcttatcttttttgtaactttgctaatggtaaaagcaataaagtaagcagaagacgaacgaagcatgttgttgaaatagtggatcgatgttgatatgggccatatatgatgatggcatgttcaatctcactgttgcttaataaattttgttcattgata

SEQ ID NO: 18 - DNA, 1575 bp, Arabidopsis thaliana
gcatctctttcccggcccaagaacactctcacttacatgatttcactgatgtacccttcttcttcctcctctctgttccaaacccaagtactacaataattctctcacattcccatggaaggtaaagccatcgcgacgtctctcggtggtgatcgtgtattgatattcccgtgttctcctcgctcttccttcgtttttacatcccggctctctagcctgcctctaaagcgtgcgtctatcggtggtgctgtctcttgttccggcgtcaatggcttgactcggtggaattccattgtttcgactcgccgactcgttcctgttcgttcaattaactcggaatcggactcggactccgacttccctcacgagaatcagcagggaaatccaggtttggggaaatttaaggaataccaagaatgggactcatggacggccaagttctccggtggagcaaacattccgtttctcatgctccaattgcctcagatcatcctcaatacccagaatcttttggcgggaaacaataccgctctttcggctgtcccatggctgggaatgttgactggtttgttaggaaacctttcgttgctttcttattttgctaagaagagagaaaaagaagcagctgtggtgcaaacactgggagtggtctctactcacattgtgcttgcacagctcacaatggctgaagcaatgcctattcagtattttgttgctacttcagctgttgtcaccatcggtctcattgtgaactgtttgtactatttcggtaagcttagcaaaactgtgtggcaactgtgggaagacgttatcactattggtggactctccgttcttcctcaaatcatgtggtcaacatttgtccctcttgtaccaaacagtatcttgcctgggacaactgcttttggtattgctgtggcagctataatcatggctcgaactgggaaactttcagagaaaggtgttaggtttgtagggtctttatctggatggacagcaactcttatgttcatgtggatgccagtttcccaaatgtggacaaattttctaaacccggacaacataaaaggcttatcgtcaatcacaatgttgctctcgatgatgggaaacgggcttatgatccctcgagcactatttatccgtgatttgatgtggctcactggttcgctatgggcaactctcttttatggatatggaaatattctttgcttatacctggtaaattgcaccagccagtcattcttcgtggcagctacaattggtttgatctcatggataggactggctttgtggagagatgcagtggcttatggtcacaactcgccgtttagatctttgaaagaacttgtttttggaccgtaatgaatgaatgtacacgccataaacgccctttgttcaagcaagtccatagagattacatgtattttcattcttttttcctaagggttataagacaactactctgtaatttcatgtatttttttacttgaatcatatagtaaaataatgtctgatatcaaaaataacttttgatattgaacggtcaaaaatcaaaatcgaaacattctcggttca

SEQ ID NO: 19 - DNA, 3263 bp, Arabidopsis thaliana
ctaataaaaaaagggaaaagaaagagaagagagtcgtgctcataaattaacggtgcagagcgattgaacaaaaacaaaaatatcttccagaaaaagccatttttgattgcgtataatgaatacacgaaattaaaatcattacttgccatatatagtacactatttctcagcttcactcactctcgagactcgtgtatgttgatttacggtttgttcaagcaaagagaatgatgaatctaggatctctttcgttgagtacgagcaagtcgagtaagccaatggttagcatcagcttttggataccgtatttcactcactggggagagagcctgctggtctgtggctcggctcctggacttggttccgggaatgtgaaaaaaggtttgctgctaaagccatcccagcaagatgatcagctcatctggagtggctctgtctcggttccacctgggtttagctctgactattgttactatgtggtggatgactcgaagagtgtgttaaggtcggagtttgggatgaaacgaaaacttgtggtgcctgagacattgaccggtggagagtctgttcatcttcgtgatctctggcagagtggggatcaagctcttccatttagaagtgcatttaaagatgtcatcttccaccacagtttcgatgtgaaagtagaaaaacctctaggggtctttatgaataagtcagatcaagatgattcagttgttgtccaattcaaaatctgttgtccagacattggagagggaacatctgtgtatgttctaggcaccccagaaaagttggggaattggaaagttgaaaatggacttagactcaactatgtcgatgattccatatgggaagcagattgcttgatccctaaagcagactttcctatcaaatatagatactgtaaagttcagaaggaagatagcatagggtttgaatctggtggtaatcgggagctgtctcttcactccatcggtagtaaacaggaatacattgtcatgtcagatggcttgtttcgagcaatgccatggaggggtgctggtgtagcagttcccatgttctctgtaaggtcagaagatgatgttggtgtgggagagtttctcgatctgaagttgcttgttgattgggctgtagattccgggttgcatctagtacaacttttaccagtaaatgacacatccgttcataagatgtggtgggactcgtatccctacagctcgttatctgtgtttgcattgcatccattatatcttagagtgcaggctctctctgaacgtctgccagaagatatcaaggaagaaattcagaaggcgaagaatcaactggacaagaatgatgttgattatgaggctaccatggaaactaagctttcaattgccaagaaaatctttgacatagagaaagaccagactttgaactcaagcaccttccagaaattcttctctgaaaacgagggctggttgaaaccatatgcagctttctgcttccttcgtgacttttttgagacttcagatcatagtcagtgggggaccttttctgactatacagacgacaagcttgaaaaattgatatccaaggacaacttgcactataacactatatgcttccactactacattcagtaccatttacatgtacaattgtcagcagcagcagaatatgcaaggaagaaaggagttgtgctgaaaggagacctacctattggcgttgacagaaacagtgttgatacgtgggtttacaggaatctgtttcgcatgaatacgtcaactggagcacctccagactattttgacaaaaatggtcagaactggggatttcctacttataactgggaggaaatgtcaaaagacaactatgcctggtggcgtgctcgcctaacacagatggggaaatatttcacagcatacaggattgatcatatattgggattcttcagaatctgggagcttccagctcatgctatgactggtttagtggggaagttccgtccatctattcctttaagtcaggaagagttggaaaaggagggaatatgggattttgaccgcttaagcaagccctatatccagaagaagtttcttgaggagaaatttggagatttttggccttttattgcatcaaactttcttaatgaaactcagaaggacatgtatgagttcaaggaggactgcaacacagagaagaagattgtagcaaagctgaagtcattggctgagaagtctttgttgctagaaaatgaggacaaagttcgacgtgatgtctttgacattttacggaatgttgttctgatcaaagatccagaggatgcaagaaaattctatcctcgctttaatattgaggatacttcaagcttccaggatttggatgatcacagcaaaaatgttctgaagaggctatactatgactactatttccaacgccaagaggatctatggagaaaaaatgctttgaaaaccttgcctgctctgttgaattcatctaatatgctggcatgtggggaggatctgggtctcattccatcttgtgtacatcctgttatgcaagaactgggattggttggccttcgcatccagcgcatgccaagtgagtccgatgtgaagtttgggattccgtctaattatgactatatgacggtgtgtgctccttcatgccacgactgctctaccctgcgagcttggtgggaagaggacgaagagagaagacagcagtacttcaaggaagtgattggtgtagatggaatccctccaagtcagtgtattccagagataactcatttcattctgaggcaacatgttgaagctccatcaatgtgggctattttcccgcttcaggatatgatggctctgaaagaagagtacactactcgtcctgcaacagaggagacaatcaatgatccaacaaaccccaaacactactggagataccgcgtacacgtgactttggactcgcttctaaaggacactgacctgaagtcaaccatcaagaacctcgtttccagcagtggaagatctgttcctgctaatgtttctggtgaagacatcaacaaaagccgaggagaagttatagccaatggctcgactaagccaaacccataaagcaataagccttctgatttcctgtttggttccacatatgttgcttattagttattatcattatcactcaaaatagtacaagctctcaacatcattgtccataagacataaaactctgtatttgttgttcctgtccaaaataaaatcctcaattttcggtgttaactt

SEQ ID NO: 20 - DNA, 2981 bp, Arabidopsis thaliana
gtgatcctcacattttcaatcactaaatcaaatcgccaaaacaaaaacagaaaatcaaatataaaacaatgaaaattacgtttctgtcattcaccttctgaaaaatatcttacgcttcttttttagtatatactgattctccgtagtatcttctcccttattaagctctgaagtgttggctcatctctttccagattccggcttcgaaatttgggagataagcgatggcaaacgccaatggaaaagctgcgactagtttaccggagaaaatctcggctaaggcgaatccggaggccgatgatgctacggagatcgctgggaatatcgtctaccacgccaagtacagtccacatttctctccattgaagttcgggcctgagcaagctctctacgctaccgcagagagtcttcgcgatcgtctcattcagctgtggaatgagacttatgttcattttaacaaagttgatccaaaacaaacttattacttgtcaatggagtatctccaaggtcgtgctttgaccaatgccattgggaatttgaaccttcaaggtccatatgctgatgcactgcgtacgctgggttatgagcttgaggagatagctgagcaggagaaagatgcagctctaggaaatggtgggttagggagacttgcctcgtgtttcttggattcgatggccaccctaaatctgcctgcttggggttatggtttgaggtacagacatgggttgtttaagcaaataatcacaaagaaaggtcaagaagagattccagaggactggcttgagaaattcagcccatgggaaattgtgaggcacgacgtggtattccctgtcagatttttcggcaaggtgcaagtaaatccggatggatcaaggaaatgggtagatggtgatgttgtacaagctcttgcttatgacgtgccaatcccgggatatggcacaaagaacacaatcagtctccgtctctgggaagcaaaagctagagctgaggatcttgatctttttcagttcaacgaaggagaatatgaattggctgcacagcttcattctcgagctcaacagatttgcactgttttatatccaggagatgctaccgagaatgggaagttattacggttaaaacagcagttctttctctgcagtgcttcgcttcaggatattatatcaagatttcacgagaggagcaccactgaaggcagccggaaatggtcagagtttccaagtaaagttgctgttcaaatgaatgacacacacccaactcttgcaatacctgagctcatgcgattgctaatggatgacaatggacttggatgggatgaggcttgggatgtgacatcaaagaccgttgcttacaccaatcacactgtccttcctgaagcgttggagaaatggtcacaatctttgatgtggaagcttcttcctcgtcatatggaaataatagaagagattgacaagaggtttgttcaaaccattcgcgatacaagagttgatctggaggataagatttcaagtttgagcatcttagataacaatccacaaaagcctgtggtgagaatggctaacttatgtgttgtatcctcgcatacggtgaatggcgttgctcagttacacagtgatatcttgaaggctgagttattcgcagactatgtctctatatggccaaacaagtttcaaaacaagactaatggcatcacacctcgaaggtggttacgtttctgcagccctgagctcagtgatataatcacaaagtggttaaagactgacaaatggattaccgatcttgacctacttaccggtcttcgccagtttgcggacaatgaagaactccaatctgaatgggcttctgcaaagacagccaataagaaacgtttggctcaatatatagagcgtgtgactggtgtgagtatcgatccaacaagcttatttgacatacaagttaagcgtatccacgaatacaagaggcagctgatgaacattcttggagtagtatacagattcaagaaactaaaggagatgaagcctgaggagaggaagaaaacagttcctcgtactgtcatgattgggggtaaagcatttgccacctatacaaatgcaaaacggatagtgaagctggtgaatgatgttggtgatgttgttaacagcgatccagaggtcaacgaatacctaaaggtggtatttgttccaaactacaatgtcactgtagcggagatgctaatacccggaagtgagctatctcaacacatcagcacagcaggcatggaggcaagtggtaccagcaatatgaaattcgctctcaacggttgtcttattataggaacccttgatggggctaatgttgagataagagaggaggttggcgaagaaaatttctttctttttggtgcaacggccgatcaggtccctcgactgcgtaaagaacgagaagacggactgttcaaacccgatcctcggttcgaagaggcaaagcagtttgtcaaaagtggagtgtttgggagctacgattatggtccactccttgattctcttgagggtaacacaggttttggacgtggtgattacttcctggttgggtatgacttccccagctacatggacgctcaggccaaagttgacgaagcttataaggaccggaaggggtggctgaaaatgtcgatattgagcacagccgggtcaggaaagttcagcagtgaccgtacaatagctcagtatgccaaagagatttggaacattgaggcttgtcctgttccctaagtccttataccgttcgcatagacgctacctgcctttataaaggcatctccacatccacgcagctccaaagtcgatatatatcatatagatctcataagaatataaccaaaataaaataaatgataaaccttttcgtatgtatatgtttttgatgtatgcaatattttgattttgttcaaagaaaacgttatgtaaaatgctatatgaatgaatgaatttgggttagtgatt

SEQ ID NO: 21 - DNA, 2136 bp, Arabidopsis thaliana
ctattctcgtggagcaccgagtttgttttatattagaaacccgattgttatttttagactgagacaaaaaagtaaaatcgttgattgttaaaatttaaaattagtttcattacgtttcgataaaaaaatgattagtttatcatagcttaattatagcattgatttctaaatttgttttttgaccacccttttttctctctttggtgttttcttaacattagaagaacccataacaatgtacgttcaaattaattaaaaacaatatttccaagttttatatacgaaacttgttttttttaatgaaaacagttgaatagttgattatgaattagttagatcaatactcaatatatgatcaatgatgtatatatatgaactcagttgttatacaagaaatgaaaatgctatttaaataccgatcatgaagtgttaaaaagtgtcagaatatgacatgaagcgttttgtcctaccgggtattcgagttataggtttggatctctcaagaatattttgggccatattagttatatttgggcttaagcgttttgcaaagagacgaggaagaaagattgggtcaagttaacaaaacagagacactcgtattagttggtactttggtagcaagtcgatttatttgccagtaaaaacttggtacacaactgacaactcgtatcgttattagtttgtacttggtacctttggttcaagaaaaagttgatatagttaaatcagttgtgttcatgaggtgattgtgatttaatttgttgactagggcgattccttcacatcacaataacaaagttttatagattttttttttataacatttttgccacgcttcgtaaagtttggtatttacaccgcatttttccctgtacaagaattcatatattatttatttatatactccagttgacaattataagtttataacgtttttacaattatttaaataccatgtgaagatccaagaatatgtcttacttcttctttgtgtaagaaaactaactatatcactataataaaataattctaatcattatatttgtaaatatgcagttatttgtcaattttgaatttagtattttagacgttatcacttcagccaaatatgatttggatttaagtccaaaatgcaatttcgtacgtatccctcttgtcgtctaatgattatttcaatatttcttatattatccctaactacagagctacatttatattgtattctaatgacagggaaactttcatagagattcagatagatgaaattggtgggaaacatcattgaacaggaaacttttagcaaatcatatcgatttatctacaaaagaatacttagcgtaatgaagttcacttgttgtgaatgactatgatttgatcaaattagttaattttgtcgaatcatttttctttttgatttgattaagcttttaacttgcacgaatggttctcttgtgaataaacagaatctttgaattcaaactatttgattagtgaaaagacaaaagaagattccttgtttttatgtgattagtgattttgatgcatgaaaggtacctacgtactacaagaaaaataaacatgtacgtaactacgtatcagcatgtaaaagtatttttttccaaataatttatactcatgatagattttttttttttgaaatgtcaattaaaaatgctttcttaaatattaattttaattaattaaataaggaaatatatttatgcaaaacatcatcaacacatatccaacttcgaaaatctctatagtacacaagtagagaaattaaattttactagatacaaacttcctaatcatcaaatataaatgtttacaaaactaattaaacccaccactaaaattaactaaaaatccgagcaaagtgagtgaacaagacttgatttcaggttgatgtaggactaaaatggctacgtatcaaacatcaacgatcatttagttatgtatgaatgaatgtagtcattacttgtaaaacaaaaatgctttgatttggatcaatcacttcatgtgaacattagcaattacatcaaccttattttcactataaaaccccatctcagtacccttctgaagtaatcaaattaagagcaaaagtcatttaactttcctaaaacaatg

SEQ ID NO: 22 - DNA, 995 bp, Arabidopsis thaliana
acctttatatgacgatggttgttaaacgaaataatttaaatcaatggagtttttataagtttgtaatgcgtttgtttttgttatagtaatattgagttggatctttgtttacgggacgtagaatactaaataatgaaaaaaaccttctcgatgaattaagggttttatgaatttgttttgtattgaataatatagggatggataaagttttattattctaacaggttactttattaggcatttcttcggctcatgtaactcttgtatcgctgaaactatgtaatagatagaagaacctaaaaaaagaaagaaaacaagaaatgcacatagcgaagctcaaaagatgagtgttctgctagcggtaatgttgttattcagttgggtcaaatgctctaattgcaaatcttatttgggccttatatagactcttatgtgcatatggtccagcctatttgggccgatgtgtttgaagatcatttgggaaagtcttgcgcaaggagaacttacttgggacttgggagacttggagtaacaaattcgtcttggagtaacaaatacatgtcaacttattccacaagttctttgtgataatgatatacatatttaaatgttttaaaaaagggaaagaataaacaaactctttgtgactttttgggtatttatgattcttttggtccttctcttgtacacatttgagtcctatcttatatggtaagctttcaaatgcaagcgtgttatttttattcacacttgttctatcttaatagtatgttgaactcttcgcctttggcgattcttcccggaaaagggaccattgactttcagatacacttcttttttttttttataataattattatcagttcaatgatttttcaatatggactctcgttctcagacatttatttctctcagtcttacaatataaattttcattcttaccatccataattttgtattgtcttctccacagatctattccagctcacgccatg

SEQ ID NO: 23 - DNA, 1834 bp, Arabidopsis thaliana
ttcgatcttgtatttaaataatgttaatactctgttttaatttgtgtatcctgatttttttttctttttgaagttcaacaaatatatcaaaataactcagaaccattactattttttcttagttcatcaattctttactacacatagaaacgtatttatcttgtttgatctactttgactctatatatgtcatgtggcatctctggtcattgctagtcacaggtaaaagtaaaaattgatcaaagataaagagtctttcatggtaaaaattctcttgtaactggtggagatagtagatgtcaattcgtttgcaataacttacatttgcaataacatgtcagccatatttatttaaatttccatgcatttgatattattttctctctaatacatatatgatgtgttacggtcattctaaaaatccagttgacagcataatgaagctggtacaccatacatgcacttgattatatatggatgttactgccatgattgatgttttgatggaattagtgttaaaggatggaccctcactaacgcggttggaaattatgatcaaactcttcaatgtcacttatcaagagagctaatgactagcacgtttagttgttctgttgtttcttatggctgcttaatgtctccatcaaatatttagacattgtggctagtaaaatgccatctaccttaatcctatatataagtataactagataataatccatatttttgctgggtttagtagctgatacgacgtttatggttgttattgagtttgaatacaaaatatagagtattgttggagttatattgatttttgttcatattagttaacaaataataaaaaaattaagaaaggtttttgaaaatgcatcttctagaatatatgtatattcgaaaaagtcacatctttaattgacatatgttttgtttgtttgttttttttactggccacacaaattgacaacaatggtcatgcatgaaatgaaatgtttgttgtcaattttttttactaacttgtaatatcattatgaaatgaaatagaaggtatatattacaaaatattacctaaaagtagagcaatcttagaaaaaaaaaaaaaaaaaaaaaaaaaagaaaaagaaaaagaaacaagattacaatgcatttaaaaagagatggaaagaatccgagctatcgaatccaaagaagcatcagcttcctccatctgttcttgtatcgtctaccagagatggtgttccggatctctcgatcaatattcttaaagatggttgttggagggatcctttggctattatgagaacattattcgtttatctccagatgtgatagacaaagggctgtgtggcctgtgagaccgatggccacttaattattggttttttgtcaatggttgtgtatgcatagaaattcccacaaccgtttgtggcttaacacaatttaccaggggtttaagtggttaaattgatacatgtagatctaaagttttatgctaatataaattagttttaattatataaattttaactacgctcatgacacgtaaatggtagaccaatatgtggtgctctattaactaaggggtcttcattattaattcataaagactttctttactatacaagacttgtcaaaaggaaaagtagtattttcgtactacgtctacccctctcacggatatgtgtggtcgagcagtcattatcataatgtggaattttgaattgagcgaggtttcaaagttcaaaactatcacaactagtcttgatcaattctatataagatctgtgatcttggttgaagaaaagaatcgtcgtaggttgatatttaacaaggaatg

SEQ ID NO: 24 - DNA, 3538 bp, Arabidopsis thaliana
ttatttaataaaaatataggcccgcgggttaatatctagtactatgcatatccaaaaaattttacaaatttatgaaacaacaaaaaaacacaaacccaaacccaacgatcaaaacaacaacaacctttgatttttcttttagcaaatctcatgtgtagcaaagattattatgcaaatcatgtcacaacccaaatttcgtgagacataaccatggataaaatatacaaaataggatattagaagggaaattgcaaatgccagtaaaatttctctcacctattctcgacaaaaaacattattctgaattgaaataattgattgatactctatacaaaatggtcatgtagaaagaatcatccttataaacaaattaaaacatacctaaaactggagaatatgatatgttaattaggacagctaggaaaaacaaaatattgtaatcatttctaaaaaagcataaatatacaatatctcttatacagagaatttggtaaaatatatcttatacatagaattttggtaaaataaattttgatataaatctattacaattagtgacaaccaatcatgatatttggttaaaacaatccatgcatatttgttaatccacaaaaaaagtttaaatatctaactctgaaatctctaaaatctttacacctatgcatctaccaataatgattctgaaagtttcagaaaaatgtctggaaatatataactgtgttgggttttcatttgaaaattatgatctcttactagtaataacgtcatggaaattgcaacacagaaaaagacttataaagttttctgatatttttctaatttaggattttctttttaaaaaatacaaagaaaaaccgactatagaaatgttggtgtaaattaaacaggaggaagagttttatccaataatacagtatacaagtaacaagatgaagaacctcgggaacttgatacgtttgagggttaacagtgaatcatattttttatataaccaagtcataaactagagaaaccatataaattgaacaaacgaaaaaagacaatctcacttccatggttagtaatctttcatttagaaagatcttaagggaaatatataattgtatattctctctctataccacacaattccgatgaacacacaatctggtatattgtacatttgtttataatgcttggcacgacggcttgtgataaggctttatcgtctcataaaaggaaaacgtagtcattcgatcatctcccaaagtttcaatcttctctactgtcttcgtttatttcaaatgattatgagttgattaattattcaaaacacagaagatctctctatacatatatatatatatatatatagaaacctttcaaaccatttcgcaaattggttgtttctcactttctctagcgtaaatctcgatgagcttaagttaaaacttaccttcagggtcatttgttttgttatgtgacaatcttctagattaatattccacttactacttcctgcttaaaatatttagttacatcacatgaccatgtaattgaatttatcctctttataatataaactacgaaaatctgaagaagaaaaaaattatcgaaaagagaatcatattctggtactagcaaaataaatttggtagaagatatatatatatttttctatatgtaaacttcaaaattaatgcctaagatatgctaaaaatttgcgaaggagtcagggggaaagcttgagaggaccaatgcatggcattgcttttactgacagtaaacagtgtcacgctcacgacccattcttcccgttccatttggttttatttatttcaaagtttaatattccttttgtataacattcaaatcttcacatgattgattgtgtgaaaaccccacagattttactacaatagggggagttgacttaaaatagctattgatgtcgaaaaaatgtattttagttataaattatactaaagaaaatttttgatttgtctgttgtttaagcatatgtattgttaaacttaaaaaaatatgtattgttaatcttaaaaatgtaggagtacacatcaaatactcgagcataatcaaaaccgtattcatagaccgatgtgagaatcaaatagaagataatgtgattttttaaaatatcgtatctccaaatcaatcacttagaagataatgtaattctttatgtgctacataaataaatatatatatatatatatatatatatcttgtatatatgtcttgacaaaaaattgccagtcaaaaaccatgactgaatcaaactataagtcggattgaatcaaactataagtcggatgagtattaatttccattatgtttctatactttacaaaccggaaaatagatattatagataccaaaaaagtagatttgtgtatattattagaagatttggaatttcatcattatcaggatctaaagtacttccctaattaaatcatgtcggttgaaaaagctcaatgaatgtttgaaatttggaaagtttattaaattcggatcttttttttttgtttgtcgtcccaaacatttttattttattacaaataatcaacttatccttactactaaatcatttcatatctttgataccaacaaatcatttcatattctattttgatgtttaagaaaacactatttaccagttacaaaatattataaggattgttgtttagaaaaaaaagtacaagttgaattctttttgtcaaatataaaattgactttttaatatataattgacttattgaacatgattacagaattaatcatctacaaaactttccaagtttataataaatacatttcaaagactattagttcttcttaaaatatttctaaaagtgatcaaagactaccacatataattcagaaaaagtagaagttgatttctttttgtcaaataaataattgacttaaaatagtttggaaagccattgaacttgattatagaattgataatgtacataaaaaaattccaagtttataataaatacatttttcaaatgctatatcagttcttcttaaaatatttcactaaaaaaacactcaaatatagaataaatttattgaataacataccaactgtaaaacagaatttgacaaaaaaaaaaaaaaaatgaaatgaagatgaagacaaaaataaatcaccagaggatcttatgcaaaaaaatatatgaatacacaataaaccatattgatatttttaaaataaaataaaaacagaaaaatatcccaacaccgcttttcaattaaaaatcttccgtcaccattgttgtcatcttcctctctcgtgaatcctttttcctttcttcttcttcttctcttcagagaaaactttgcttctctttctataaggaaccagacacgaatcccattcccaccgatttcttagcttcttccttcaatccgctctttccctctccattagattctgtttcctctttcaatttcttctgcatgcttctcgattctctctgacgcctcttttctcccgacgctgtttcgtcaaacgcttttcgaaatg

SEQ ID NO: 25 - DNA, 2223 bp, Arabidopsis thaliana
gatttcaactaagggaatggccagtatatatagaagaatataaaatatcggttttatatccaccatacatttcctatatatatatattatcttttgtcaacctccctatatatattttatattttcctgtaagttttagtttcgtatatgatagggttagcaaagtttttgtatatgagatatacatatagctttcttgttggactatataatccctctagatatagattttctaatttcggcagattttgagtaaatatccaaaaaaatctcactatcttgacaagattacaaaataatcttttaatttaactatttaagacaacatgtataaaacctatatcttaactaaaaactctatattattgcaactatataatctaaatgtctattatcttaaataaataaaaatgccactaccactttttgatattataggccaagttattgatcagtaaaacgaggccggcacgtaggaactaggaagtcaaaaaataatatttttgtgatggaacgtaaattcaccaattcaggaccttgaaggggcaagaagccaactaaaacaccagccccaaccctagtgcaaattaccattctatataatgaccccatctacgatttattccttctatttccctttacgtaaagtagcgacgttcgtgaattcctaagaagttagactaacaggtttgtgtggttcttaaatttttgggtaaaaattaggaaaaaagtttatattcctacccaaactttcggcaccagagacaaattaggtttgttcagaaaatcagtcgctcatcgacagacttaacaccacaaaatcatcagaatttctttgggacaaaatggaaaatgttcttcacggctccacctaccaaaatttcgatatcaagctcaaaagcatacacaaactataactaattccatagttacgtaatcttaacttcgaattcaacaacacatgcatgcatcgactgaatcttcaacaaatgcaatcaaacacacaaaattgctaccaaaaaaatatgattttttttttatgatttcaattttcatccggcacttagtccaaaactttttttgtgtgtcaacattttttaaaaaaatctttaaacggatatctgatctaagagcatgttcataggtgatacttacaaaatatttttaagaaattttttagtattatttataatgtttgtttaataaatatatataagattttttgcttttatcaaatgtgaccaatcagaagaaaccacgtcagatgatactgatatgacaaatatgatactgatcaaacatattctaattgctttactaatataaaaataatttttggacttgtgatactctaaaaatatcacccatatacatggtctaatatatggatcgtaaaaaactcatatataatattaataagtagtagaagagcgtagaccatgtcctgggtcgtcgtccaaatgaccacaagaagatttcaaaacagaggaaaatatttctcattaaataagttttcctgacgcataagataacattattacaagattcagaaaaagaaaggtgaaaggataatgtttctcctactatataagatgtgtacatctgaaaaaatatgaatatatttgtaacgtttgactgttattacatgattaatacgatataaatattaacattttttttcaaaataaaagtaatatagtaaggaaatgaaaagaggcatgaagcatgcctctttttttggtcggctgccgtttacaattgccaattgcgatagttactcttcttgcgtgtacgacttttgtttttttttacatattcgccaataatttgacgttttctattagtttgtttgatactctgttgtcttgctaaaactcaataaaacattaaattactttcttgaatgaagctggaacaaatctaacataaatagaaaatgatgggcaagttgatgttattcgtaaatttatttagattatattatataaaaagcaatccaattatatatctcatatatacaatttcttatcttactttgtcaatgtcatatacgtaactaaaacttgcggaaatagaaaatgccacgtgtatggtggacataatccgaatctctctctttcttctataaatagtggccattcccattggttgaaatcacaaaagcatcataagaagaagaagaaactacaatagttaatcaatcaaagagaagtaagagaaatg

SEQ ID NO: 26 - DNA, 895 bp, Arabidopsis thaliana
attggatatatgtgtgtcccaaataaaagtaaacaagaatgtagaacacatcaaaataattaatatcacttatcctgcaacaaaatgagaaaacaacaaaatagatcataaaatatattattcaaaattatacttagtaaatttaatggtaaaagatcaaatatgacttgcttttcaaaatgttaaaacagtctcgaatacgtaaaataatttgatcacaaaataaagtatatagataacaagattttctaatctatatcaaattggaaaatattttttcataacagtgaaaagatttttttttaaaagtttgtacattgatttttgattcaaaaaccttagttaattaaaagatctcttaccaataaaagtttctgaatgggatttggtattgaactgtgtccttaaatccgggttttagttcaaccacgagtttaactacataaacgatttccggtcagcctggtcctgacgggccaccaattttcaaaacattgacgaaagcaagcccaataacatcaaatataattaacaaattttagtttgaatacggttactgttttggaaattgatacattccgatcttggactaatctataatccatgggctggctattttgcagtttcccgtaaataaaattatgaaaacatgattgatttctcgttggtagaccatgacatcacaggacacgtcaacattccaagtataaaggacaaaactgtccatttagattccaccacgtgtcccgataacgtttccgtctgtatctctctcttccgtcttctactcttcttcttcttcttcttcttccactcacaatctatcgagctaaaacactgaagcagacattagcgttgagaaaaaaaaaaaagtaagaacgagagagagaaagcc

SEQ ID NO: 27 - DNA, 1909 bp, Arabidopsis thaliana
agtgggcttgtctcttaaagacaaggtggagaagagaaatgagacttatatttctatagattttttgattctgtgtttcaatagtttggaaaagtaaatcttttatagtaatgctataacgcatatatccttataggattgttgtaataatgacaagactaacgttaataaaaccgtaatcagtttatcatcttatctttatatcatttattttgagttgtgacagatttttcaatttcttgtttctgtagtatgacttttataaatgattaactgaataacaagagattataaatggttttgaatgattttatatgtatttcatgtttaataacataggatatagaaagatatttaattagaaattattagttgaataacaggagatctaaaaaaccaaaagggatgattggttgttgctgtaaagtctttccacagcccaaaatatgtctacaacattaagactcatccagtgttgctttagacagtattacaaaagctaccggctatatcccaaatatagcaaaatcaaatgtcttggctgtacgttatcaaaatttccacatttgtattgtacatcacgtaaagaagcctgtgaactttctttctctctctattttttcacaattgttttgctctttgcactaaagaaaagaatatctaatgacaaaaattaacatgactataatataaagataagtaaatacttttacactaaaattttgtagcactaacaagtgttttatgtttttaaatgaatatcgtattcgttttattaaataaaaactttaattcagtttgtaaataatattatatttattaaatccaaaaaaatattatttaaatccatatacagtgatgatgatgctatcacgacaaattagtccaaaatgggcagcgtatattttcttatatgggcctaaacaaaggaaactaactaattataatcaacttatgatcatcagtcaattatgaaaggttatatttgacaggagattgtgtgtttaaaaccattttgagtatatctgttttgaatcatttgagacaattgtgatttttgcttgttctgatcccattcttttagtctcgtgcatatgtgatcttgtattgtctaataaggattatgatctgtcccctaaacttcaaaatttggaggccatgatctgatttttgaatgtggttcttatagttttttgcttgatattttgcagaccaacaaagaatgatggtgaatatccatctaagtcaaatacagattccatttctcttttaatacaaatcaaataagaaaactgaagcttgcaagctcgcttgtactaagtttctgaaagtttttattctcgactaaataatgtccaagatggaagcaagacataagctccattgttgatagaatggagcttatgttggcttttgtttttgtttcaaaatggtttttaacttatgtgtgattgcttagccaaatgtggactctgaagatggttttcagttttagatatgaatgtccttgttactattgttacacagtcacacagagtttacaaaagatcattcatcattgtttataagagaacaaattgataacatatatctttttgatgtggatggatttgttaaccctctagaagaacacccaaatttggctacaaagttgtcaacacattgttcttcgttcttctttgactttgactaaaaataaaaataaacctcggcctaagaaaatagcgtgttcacattggatgattaaggcctggaccgagtatcaaataatatctaaatgggcctagctaagcagttacgtaagctaaatttagggtttgtttttgccgcacctataaaataacaacacagcttcccatttgtaatttctagggttttgcaaaaccaacaccgaagatccaacacgatg

SEQ ID NO: 28 - DNA, 3151 bp, Arabidopsis thaliana
gggctttttgaaaaattattttaaatttttttgacaaaagttctcgttcatatttgtggatatttcccatataactttttaatctccaaaagaccccaaacttttggattgattcttaaatccacccctgacctttttaaaatagtatttatttcctaccattgatgatggcaagcaatagaaaatcatgaaagataaaaaaaaaaaggttaaaaatctagtaacaggcaaatactttgaaaaggagttgaaattttgtacataggtttaactcttctattaaaagaaacaaaaaatctctatgttttaaaatgtactactaatttataatttaagaacatttttctgaaaattttgctggttgtttaacgaaaagaaaaatatacaaatagtttttggctgtaatgctacgccactactcacttttccaaggacgtgcatacaaattaccgtaaacggattataggacttgtgttgactaatgacacaattcttttttattaaaagagattagtctccttgaagaaaaagggacacaaattattcgtaacatgttaatcaaaccaaggtcttaagtggctaccaaccgcatacgagtgttgagccgagcagtgctgtgtcgagccgggctggatttggcatttggaatatatgaaatgggccaacaacccaaaagataaaatttcgttacttggacagtaaaaactaaacatgcatgtttattagcccgtaatctgcataatcacaacttacgaagaagacataagaatcaacaggcccatcataaagtgggactaacgatttggtttccactattatttggcccatatattgttaatattgtacacttcaaatattatttagttagaaaaaaaaaaaaaaaaactcttgtaaaactggatggttctagtttagttttaaaaaatggttagatgataacgatattaatatacgataaagtttatataaaatatcttttcataaccaaatttacatgacaactaacattcgggtaagccatgtaaaagacgagatagagattttgaccaaagaagaaaagtagagagaacatgcaaaagccaaaaagaaaacatagccaaatggttgagtggtgtagatggaccgaaccaacagacatgttctcagttcatataccaaccgcctaacattacagcctgtccccatcccctatctttatttttctttttggctcatttagcttcaactttttttaataggtgtgattagaacatactaaaaaaaacatatgttttaacttatattatatgaataaagtgttgtttttaatagtttatatttctattacaagtacaatatataggatattttggttaacaattttaaatttaaaagatattgtgagtacaaatcattaagcttaacacaaatgttcattgcttcttaattcttacaatcggaaaatttggacaaaaaatctagactaatctccaaaacattgtgaaaaattgggggttttttttatcatttaaattgtctatagtggtcaatcattattaaaaaaaattgttttttgttgaaattttaaattataaaaaaaaaacacgtttatacataaaatgcaaaaacttttcttaaaccaaattacagagaaaggctatcaccatccttgtcttgatgccaaccacacttgtaatcccaaaatacatacttaaactatactatactaacatgaattggttatacgattatctatctagttagctacataattcatataattagttttttaaaatatacttatgtaatgatagtaactaaaaaatatactatctcgagaaatccaaccaaaatgctatgatagtatataaaataaaatgaaagaaagaggaaccaagttgtgaaaccatttaccaagaagaagaagaaaaaaacaagatggtcgagtctcgtgatagtatgaagctggggccgaaaccacgaatcaaagaaccatttgacgatgcaagtggtcatcgataaccaatcaatagaaactccaccgcctcaaaatatcatagtgctatttaatttttcatactatcatttattgtaggctataacatatttttgtattgtagatggacggctagtacacgcaagttagtcaataaacttttaacaaatggcatcttgacatgcttaaacacattttataattcgaacctcactttgcctactttacacacgtctacttatagtagatgagcatcaatcattgaaccgttcgtataatacaattttttttttgtaatacgttataattagaaagtaatggaaaataagttgaaacacaaattttcaacggttttactaagatttaattttacaattttcaaaatataaccgaattatttaaatcgtattaaatttataattaattttctaaaaaaaaacacagattaaaaaaatatattaaaactaatcataaatatattaatttttgtaatttatatttttttataactttaaactaataatattcaattatttatcttaaagtttacaaattatcaataataactaataaataatgcataaaaactttttaaaaattaactttttgaaacaaatttttttctaaaaaattaaataataaagaacagaaaaaatattattcaactaaaaatattggataataactctgaaagtttgcatttttcaaataaatcattgattttttttgtttgacataaaacattaatttccccttgtaaatcacacatttaaaatctaactatatttaaacaaaaaaaaaaagtccaattttgactttaattatataaaaaaagcaaagctaattatgggtcaaaagactcaaaggccagaattgacgcagccgtttatgagaagtgagaagacaatttcggagtcacttcctttatttcctctcttccttcactctctcctatataaaccttcctctcctcttcctctcttctcatctcttcaaaccattttcgaaagccttgagagagagaacacagacgataccaactttcttcaatctcgttgccgcagtataattatctcattcctcggatatatctctccttctgcggcggcgacaagaagctacaagaataaaaagtctgttttctctcttttcaagaaaccacttacttcgaaaatg

SEQ ID NO: 29 - DNA, 2050 bp, Arabidopsis thaliana
aggagaatgagaaaatggattcgtataaatagtgaagagaagagacgagtaaaagaatttgaataaagaagagagacgtggtcggacttggcccaccaactttgtcatctatgatgcaaaatatttaacaaataacaactaattgaggttgtaacgtgcatttgtaatgcaatacattgagaaaaaaacaaatattatacataacaaagttgagtttttttcactagttgtttatttaagaattagaacaatagtccactgttttttaacaagtccatagcgaatgttggaaacttattttaaaaaattattaattggaagtctaaagaattacgaaaacaaattgtatttgattaaaacaaagtacaaattgcatttaaaaagaaatagtctatagaacatggatgatccttccatataaagtttgatcatgcacatagtatgacaataatcaaagagcggggatagctgtgaatctttagcagcgaaaacttcaaacatatatttcatgaatacaacaatgttataaaatgaatttcgtacgacataataaatgttatagtaattcttatgtttgtccaaaaaaaagaaagtaattcttaggttaaatatataaaaacatgacaaaagtgtaactattaaagatatttgatgtactacatatatataacaaattttatcaaagatgtacatagacaactatgttcttaaaactccacaaacattacggcagttagaatttaaatttatcttggaatattgtttgtatattattaacaaatgtaaattggtgtaggttttcagattttcataatgaaaacagttttaaattatggttacgagataattttgacgaatatgaatacatatatagaaaatgtttgggcctatatctatgaaaatgtttgggcctatctatatctatgaaaattgtatgagataccaacaaaataaaatatttaaagtagcaaaacattgtgttacttttctattatcttgtcttaaataaataaaaatcaattttttttttaagaagtaacacaatgtgaagaatacgtgtgtacttttttttttttggcgtaaataaaattagtaaattaagacaagattaatagaaaagtacacacgtattcttcccattgtacgttaaaaagctcgtgaattcacatgcatgaggacgatttgggtaaccaataagttgactatatttttgtatgtctttctatttttcttcacatggaaagaattttccaacgaaaagttgaaggggagacaattaggaacactaaattgtcaccattaggctcttaagttttaatttggcaaccaatttcattttctctattctgtatttttggattgggaccgttttatttgtttgtgtgtgtgcaacttgagctcttcttctgtaacacgcatgtgttgcgtttggtttttaatttatttgcctacttttgcgttttcaacgtttgttaccacatttgaactataaagtccaattccataccgatcctctacaaccatataaatgtgctacaacaaaagttaatctttgatcttgatcaactcaaagatgactatacatgattagttctcctaaaagctaatgttgtagagtgcaattgccgacaaacctccaagttttgtgtcaacttgaatttgtacttaattgcaatctttggaacgtgttcattcgaaaacgagaataaaaagaaaaaaacatgactacgtctataacaagtaaacaaaagacaacagtgaaaagaatcttatcaaaagttcaaaacgcaaacgtggctaaacctgtacccaaacagtcccataagaagccagaatcaaagtacactgtgtatataaatgagcccaacaataaccgaataaacaaaaacaaaaaaataacgttgccttgacgtgtcatcttactccatcccttccttttgcttcttctcctttacttccttttttttttctttccatcattttgtcacaaaccggaaaaaaaatctcaccggagaaaaaataaatctcaccgacgacgaattcgatcatg

SEQ ID NO: 30 - DNA, 993 bp, Arabidopsis thaliana
tatatagtgaggtatagaatcaacagaatatctttgatgatgcatatcgcaacttataatattcctcttcttgcttagatcatgtttggtttcgtgttgggaatctcataaatcatatatagatgtagaagttatgtttatctccctatggtaaacctcttcttagtatgcctcaagatcacgatttacatgctctagtacccgatccgagaggattatctaccaaaggcccataacttgtaatccaaatagaaatttctttgcgaagctaaaactcaagttttccaaaacaagttgttagaatgccgactaaagtttctttatataggtttaacaagaaatgtataacctaaagatttgatcccatggcataaaaaaaataataattaaaaacatggaagcaaaatatatacaacagatagactatgatcttgcagtctaaaggtgagaccactctttgatggaccacaacctcaactttccgaatcagccggcttttgtttcaggagccaagagaaagacacaacaaccaaatcaaaaatgacaacacggctccaaaagaatatggagtttggcaaggcttacctttgctataaataagttgagcattggacttgaaataactttgtctaatgaattcttacttgtattttataagacgaaaacgtaaatcttgcattaagaattcatacaaattttcataattgataaaattcttacattatcgtactagaaatcggaaatgcattccaccattatgcgtgttagtactaatcggagttttaacttgtactaaatttaacaaatatgacggagagagttgaaagcttatcacaatggcatttaccgttttgcccttgtcgttgtaatttcaacatttccaatttcttaacgcctttactgacatcgaccccaaaaagaaaaaaaaaaaagaagaagagtcaaacctgagaatctcatctctctctgaagtagaagaagaagaagacgatg

SEQ ID NO: 31 - DNA, 1969 bp, Arabidopsis thaliana
gaagttgttggcttttgaggttaagtagtaagaaccataggagattttgagtcagtcatattcacaattctttattttgcttggttttctggtccggtttggggttcaaattctcaagtctcagtggattacataagggatcacagtgtatctataaacccacgtatcgctaatggtgatctacacctgatttagatagaggagactccacaatgttaacaaataatataaaactatagtgctattagtgaaaatgataccacaagttgtcattagcagagaaagatcagtatcaatatccaatataggaatgaagcgtcactgtcttaaagagtcattatcgtggagaaccacatcactaatccacttatgtgtagttagttgtttgtaatcaactttttgtattttcttgtgtaatccaacttttgatgtattagtttaagctgtattacttttaaaagcttgcctttattttccttgtgaaattctgcataaatatcaaaagagatttatgacaaaacaatgttgctttttcatcattaacggttgtggtattttagccaatcaaattctttagtttctaattttaaagttgtgtttaaaaacaaaaatgtaccactatcgtcgacataagaaaaaagtatctaagcttaaaacgctaacattcataccaatttaaatttatatcattccaaagttcttcaaaatcccttagtctctctatttcaatatgtacgtataaaaatgtattataagacatcgaaacgtgtcatgtgtacactgaagtcgcaggactagaaggagctaataggtggccatggtgctctacgtggactctctgtgtgacacgtgtcaaagtgattatgcatctttcaaacttctgtgataggttggttggcccgaccaagacgaccaacatactcgttgaacacgtttcgttgcatatttctgtattcatctgtattaatactaataagaaaccatttctgtttgactgtattaatgaataatgattcctgaagattttaaattatttcttccaataatatgagcatagaaaattattaagaactaattaattctttcaagtggaaagtcattattgaaaataagttagagatttatcggttagtgggttttaagtaattttaagatgtagagaaaactatgtattaatggatcattattctgtatcaaaattatgtgtagtttgttaaataatttattgatactaattaaaattggtgttattctctaaatgctttttcaatagattcaagaaacttttgattccacattttattttaatagatttagaagtgtttttaattgaacatagctagaacttttgactttcaattccattttctcataaatttatagtttaaacttaaagtcaagaaaaaatcttacataagtgataaaatttcatagatatctaaagtcaatagatattttgacttaccaattaccataagcaaattcaattaacctattaaatattcacctaatacgacgacggtggggtcaaacattagctggctcaactttcaaagccctttatcttcaggtcccacaatttagtttaacccaataacatggtaacacgtgtcagattaagctaaatatccaaaaaccttaaaaatagtatgcgttacagagaatcagatcccaaccccaagcctctatataaatcccagaatcatcagtaccatttttcaaaatccttcaagaatcgcttcatcttcttcttcttcaacaaatcgagtcctttttctgataactcatttcgatttcagaaactcgccaagaactggatactctggcggaatcagagattaagaaccgagacgtggttctgaaactgtatgaagccctgagatctcgcgacgtcaaatctgtgcaccagatcttaaccccagatctggagtattggttccacggtcctccgccgcaccagtttttgatg

SEQ ID NO: 32 - DNA, 8111 bp, Arabidopsis thaliana
ctgtgtttgtatttcgctctctcattttacttaccctctatactcatgatttaccaatgaaaatataaataaaattataatatttatcaaaaatatgaaaattgtaattggacttaggtctaaaacgtaaactttgctattctactgtgagtaaatattacaatttagcgtggtggatttgcgggaaagttgttaccaagaactcgtttccattttagtcaagcgtttaggcgaaattaagatatccgttgctaaatgtacgggacaaaaatactcaaattgtaaaaatgggaaatgaagaaagaatagaatacttttttgtgcaaataaaagaacagaatacataaagataagatgtaacatgtgaggaaaaaagtaaaataacaacaaccgtccaatgagcctatactaaattactaataataggcaaattctattgtttcgtcttttctctatgtccaccaaaattacataaattgactcaatgctcactcgttgcacacacataacacatacatatattcggattaaagtgattgtaatcttcttaagatcaaaacgtacgtaaaagtatatttttgagtggttgttgatcgatcgagacgtgagtcgcagaaccgataaaaaaaaacaagaatttaggtaaaagttaataaagaaagttattaaaatagatgccaaaccatcaaccaatgatacgtattctgagttattcgaaggtgtaatgttcgatattttaaatagtttggctatgtcaatgtgatgaattctaagcaatttaatttggttaaatatatgtcattgagttgtatatttaattttttcataaatttacttgcattattgtataagaactaataaaataatcagtgagagaaatatactatattgaacagtgacgttcttcccttcttcttaattattatcctcttttttctgatgaattcttattatccatattgaacactattgtataaaagtttctagtatatataaacatagatgagatctaaaaacaaatctttttccaatggatattttcagaatttagaactttaaagaatgcatcactcttacatataaagaagttaaaataaagagaggaagaagagtacttctaaaagtgattcgcaccggattctaccagagcaagcaactggttttgtttctatacaaaagcaatcttataatatagacatgttacttttgttttaagataattatttggttttatcctaaatatgcaccatgcataaaacaagaaccatttgattgttacctttttattataccccttatttcgtaccacttcttatgcattgtcggattatgataattattcaaattttctcttttttgttacggtagctctcgaagtactctatttagagtttgaaaatgtttttccacccatcggttgttgtatgtggaaacccaacatgatttgactagttcatatctgaatagcgtttaaagtatttttgggttgatctattcagaaatgagtttaaagtttaagctcgatttcgtttttaattcaaaactgcacacattcatataaccttgtacgtaatgagttgcgtaatcatatgttttatattattgacgaggatataaaacactgaaacggaagtaacctgaattcaattattgtttaagttaatcgtcagctaacaaattacctattttatctacatatatatatatggtggacttggatgcatgttcgttgcaaacttattacaataaatatgtattattagtagattagattttttatttaagctaacaacgactttctcaaataatgacactaaaacattatgtaagaatcttatagaaatacattatgtaagcaactttacgtttttttttttgtcaacttcatgaatacattaaaaaaaactgatttttttttacttatactgtattatatattgttctagataaattatccacaagaaaatttgaattcttgtagaacaggctaaaaaatgggtccaaagttttgaatttcatcgtatcaaataaatagaaaatgaaaagaaccttttttgttacaggaagtttaaatattttgacaggttatttggtcctttctaatagatttagaattatttttatagtgatttgcgcacatattaaaacatcacaaaaatctcatttaatttacttattttacataaaatattattatctacaattaatttaaccaagcaaaaaatataccataaaatatcattggtcaaaaaatactaaatacatttatagttgaaaacattacttaaaatgagccaaaacaaattctctagaacataatttaaaattatactaaagagtagatgtctgtgcacaatgttttctttttttcaagtaatagtaatagtaaacttccggaaatttcaaaataagatgtagatgattaaagatatataactactacatacaagaacaaggttgaataatgatcaatcgcttaaaccaatattgaataaccatcaaacaaaattatagataaacaagtgtaaacttaaccatttagaaatgttctaccaaaccatgtgaggctgtacgttttggtaggttatttataaaaatagcatctacatatatacttttacgtggtggggctatgaattcggaaattggtaccaaattttttaaataaacttccgaaaagaatatgacatttacgatcatatctccggtttcccttctccgcatttctcactttagcatttaagttttgtacgacatctaattttcacaacttaaaatatttatatgcttttagatttcctcaaacatatataattgatgatatgtaggtttcttgatattagatcgataacatctcgttttcagccttcatatcttcttcgataaatatacgaaattttaaattttctgttggaagatctcaatcaatctttggttctcggtctttaaatatcacccgataatttcatacatggtaatacatgtgaagttgaacatatcttcaaaataatagttttatatccggccatcaaatcatcatcgacattcataaccgatcttaggatcaaaacagctacaatacccaacggttagattatcaacatttatacataatcgtgggatcgtgtttgtgatcaccatcatgttcatagttaatcgaaaacctgatagaagtacatcatcattgttggttacatgtttatgcgatgacctgaaggcatcctagataagtatatgaaattgtaagtttttggttgggagatctctccggttcttcatctccaagtattattgtttttctttttatattcgagaaaagaaataaaaacgaataagtttgtataagtgattagaatatgtaactgtaccagtgtacttaaggccaaatttaagccttaacctctatgattaactagagtgacaatggctaaagtatcttccaaaaggcaaagttgactaacacttgtcccttaatgtccttcttgcgtatgtaagaggagaccaatcatggatatttaactatagtatacatctaataagaaatatactattatatagaaaaacgttattattatacataatactcttcttttttttctaaaaaagtgatacattttcggcgttgaattttacttcgtctataattaagctactctgtgttttagtatacaggatgatttagaattttacttcgtgcaaattaaggaaaaagtattagttgtagattttatttttttaactgcaaatatgtaggcatagtcatagatgagggggctcgatcgcgagaggacataaatgatgaggtacatggaaccactagatatgcagatttgactttaatatattctaagttagttgagattcagattctaattcaattattggaataatgtttaccttttatattgtgccggaattagtgtactaattcaactatgcgattagtcagcttaggcacttaagcacgtcattgtaaaaaaaaaaaaaaaacgtaacgtttaatttattaatgttcatagagacgatgtgtgaaatattattcagtattcgtaatgtaattttaccgccatatatctaacattttttttttatctaatctatcattttaaaattgtgtatgaaccaaaacctaaaattatgaaaatcttggtttctttaatagattaaaacggtattatttcccttaaacatcgtatgagtgactaacctgcttggtaccaattatacatataattctgtactaattaattgagacaaaacaaaacaaatgaattactaatgagttagcgacagaaaaaaagaaaacaagacatgggatacaaagtttttaccgctagttttttaccatcatcattttgaaacggtaaagaggttataaatttatatttcatatgatttatttcaaaacaaaaattgtaaaaagtttattaaactgtaaataactaaataataagtataaaagaaattgtaatattgctttctatcctttttttttttagtttgtcttttttttttaccgtcaactcttaccacattgtttaattgtttcttaactgtatgacaccattgcttaccactacattatactggtaaattatattaccatttctataacaaatcgtatctatatggatttataaaaacattatttgcggttgaaaatgaacacattttgcgtcggcatctacacttcataaagtgtcgacatcaacatttgcaagttgcagatgcaataataaactgaactatttgaattgtacaagtaagttaaatcatttttcttttcattggtccgcgtagctttcgagcttcaacgattctttctcttaccaaatttttccatttatttttccctcctttttacctcgttctcatttccacaaaattctacaaaaacttgtttaatttattatatatctttcttctctcttgagtttcttaactaggaaaaaaccaacaacacttgttaagtttgtttaattaagaataaatttggtagatagcaaaaaatggtaataagtgaaagtctatatatttagctagcatttagcaatgaaggcggagtaaatatgttgttaactgtcatttaacgggtgactttttctccctgccaaaattaaatcttaaaccatgatcattagacttccatatgtaagttaaacaaaaaaaattcataaatatattatttttaacgtgcacatctaatttatcaattaaaatccctaacaaaagtatatcatagcataaagccataaacccaaactcgtaaactttgttggataaattagccaattagggcacccacataagtaaagaatgttaggagaaaaaaaaactaaagttaaatgaaaggtaaaatcattcaaatgatgtaaccaaaaatattcgtttgctctcattgctattttgaaaaaaaaatgtgagtccgtatttagtcgtaatgtacaaattctcttcaatatagtaaaattagaaaaatatcatattcaagtttaaactcaactgtatgaaaattagatgggaggaatattttggtagagtttttttaatcgtatactgcaaacaagatcgttatctatttacatttttacattctcacgcagtctaattttagcaagagaccatccacactaacctctcaatgatggtgacatgttatactagtacttgttatatttcttctctctattattataactagttcaaaatgtaaaaaaaaaaaacacaaaatattttttatgatttttttactattattttcattttttataagaaaatccacaaaaggttatgttcgatatcgatttgtaaaaagacctctctaacaaactagagtccagaaaaaggctaccatgtctccacttttaatcaaaaaagaaaagaaaaaagagaacaagctatttggttggtaatgtgaaggtccaaattctacgtacgatcagaaattaaagcatctgcttttgtttgttttttcttcagttcttcctcggaaaataaattaatagaattaagtcacatgcattttgaataaattaagaaatacgagtacaaaaagtttcaatgaaaaaagtttaaatagagatcactttaaatataagtaattttttgttccgtaaaggaggcatacattaaacatgcataactgaattaattaattaatatttttaattcttttttttatatatatgcgtttgtgggtgtaattatggctatccctacgatgaaattgcgtaactaataaatgataaaataaaaaagcattgcgtagtttttttttttacatagcgtagttaaaatggtcattgtagttgttgtggttacaaatcaagattgatacattttctttcttgtcgatttcctttggagttatcgttttcaatacaaatatgatgtgtcaaataatgttatttaaaaattgatctatagtataatatttgcatttacataattacttcaaatcctacataaatctcaccttagagaaaatttacatataatataacattttggaagtttgaaaatcaacataaattattttcaaaaatatgagttaagcactgaatatctaattttttggttaaaatatttttgaaattattatgtaagtatttacctgttttaaatgtttaaatattgacctaaatataactatttgaattttaaattccggtaccaaatctagtccaaatataactcatacttaccgtttttctgaaatttgcatatttatccaaatttttctaagtcttatgaatactaaaaaatcgatatattgcccatcaaattgggaaatcccaaaattcaagaaatttgcacagctctacccaaatttttagaaggtatgggtgtagatgagctacatttgttcataaattcttagaatgttcacatattcatcatgtaaaataacaaatattaaagtatatatcttcaacaaacaacaacgggctctgcccaaaagaatcgaatctcttttgtccctctttgaattttgtattgtttatttttataatatgtgtgtagattcgacaaaaaagtgtgtagatgattcatggttgggtaaggctcgcagcttaagacttgcgttagcaacaactttgagaagagtaggtccgacgcgttcccaagccgtacaatcgtctaccacttatcttttaaccggttaagccggttcgattgcgacaacaacctatgcacatgttctttcgttatcactaatagataaataaaaattcgtgtttttgttttaattgaaacattaaaacaatatccttttatatggtctatccatttcttttttgaataattggacctaatagcaatggagaaaaatgcctctttttatcctttttgaaaaattgaaggtctattttgaaaaatggtcttgtgctaaacaaaattatgggttgaaggtctattttgtcaataaaattttaaaataatgtaatagatgatgaaaaaataaatttggtcaaaataattataaattttttttttaaaaatcaattaattctatcgaccataaaaataaaaaaaaattcaccacctgaaatataaaaataagttaatcagtgtttaatttctgcctcacacttgttatcaacaaatttcaactttacctatcagtttgttggatcaatttattaccatccaattctcttgttattattcaaagttcaaacattccgttccaatgttaactttgtaaagtagtaaatggtaagtaacaataactctaaatacctacccttacaaattaaaaattcaacgcctacataaattatctacctactagaatttaaatatataaaatcctagaataagtcaacaatcatattaatgactaaaaattaccaaaactaaattatttcattagtttaaaaaaaaaacaatttattatattttatataatattataatgtttgcaaaaacagagtatcacgtcaccttctctctctctctatctctgtatcctctcattgcactataagtactaccacaaccacgaactctaaagcatcatctcattaacaaaaataaaacacacaatctcaagattttctacttcttattacaaagattcaatcttcttgtttcttcttgcaaccatg

SEQ ID NO: 33 - DNA, 484 bp, Arabidopsis thaliana
tggtgatgtacaaaacccagatctaaatgacgaaagctcttgaaaccgagaagagaaggagaagatgcacttccttgcttcgtctccgtgcgattggtatttactttgagaatttagagacacgtgtacgatagatatcaaatggcgggaagataggcggggattttatgatttgattaagcgggagaaaatgtgaataaacgcccgcttgtttgttacgaaaattttgaaattattcaactttcaatttcaaaggcccaattaaaagcccatttgtttgttacgacgtgatagagagtagtaaccttctcgatgacttcgaaggttcttgtaagggtatatttggaaagttaggacgcttccaaaaacttttcaactctcaccttcaaggtccccaacaacgcacacactctctctctctcctctctcttacttttcctcctaaactctggcgatgaaaagtagttagagacaccgagcgatg

SEQ ID NO: 34 - DNA, 2510 bp, Aspergillus nidulans
ctacaaaaagctgtcaactttcccattcaaacctctctctaaatccttcgcaaccagcgccgccatatccgacttcctgcccagaaattgcagcgcccgaatgcagtagttgcaattttgccgcagctgcaggtaccccgctagtgcatttccttggccgtccagatctaaacagtccagcaagatatacgcagctttggcaaacgagtgaataagaacgacggtccacggttctaccaggaatgccacctcggtcagagaatcatggaaatggcgatacatcggagatgctttgcccgggtccagctcctggcctcggagtgaagagcgcgcaagggcactaactgctagtgcattatcgagccttagctttgttacaaggtcgatgtggttgatatcagagtacgaatcgcggtcgatgctctccaaaacgtccgctagcaacatcgcggctagatgccagtgaccgtctagaatgacgtaccaagactggatgcgcgaagggaggagctcgtggttagcaacgcagtcctgcataaatggttggtacttcgctgtccagtgattataaacgtagagcgttctctggatggccgcttcaaggcgggcagggctggcgccgcgatagaggagggtttggagctgcgtgacgcggcggtagaggaggactttgacgggcgttgcagaggagagagcggcggcggcctgttcgtaggtgcagggccatcgagccgctggctgagagatttgtgtgtgggattcgtgatctgggagagagtccgaggtgcggaggaagaggtcgccccatacgtcgctcttttcttgattgctcgggacctgtctcgggggctcccagcagtctaggttgatcggcgtttcagcgccgcgccttggtggagatgccgatgatatctggctatcctcatctgacaccacgagtggtcgctggtacattgcagcgcttagtgtatcgaacatgatccctagccagaacataaggcttaatgtactgcgatgctcctcactggccacaaccgggtcaagctgtgggctttcagacgacggcgtcggtgtctcgaaaataccggcgaatgtcgatgcgacagatcctcccgggagcctgttgaaagccttaccgcggcgttgcattcgtgcaaacttatgtcggaatgtataaagctgacggttcgcggtttccaggaacacgggcgcaccgtcattttcgagtagcttgtctagacgtgcacccataccgtgctgctcatcatcatccagcacactctgcgtgagagaaaagatgatattcgcaaatataacccggaatgatggaatccctgtcgtgtgctgcaaggcatggcgtgcttcattccaggcgttcctccggatggacctctcatcggcggctatgtctgcaggaacatttagcccagcccccctctgcgcatgctgcgtccattgcgacgcaaaagctacgatcgccagatgcagggctcgggctgcggctttgtcctcttccgcactcagggcgcgcccgcgtaatgaggtagatacgcgatctagccggcacacccggatgcacatcctgtttgaccagttcgggccccattccgcccgctgcttgggcggcaggtagctgatctggtcggagtatggacaattgtgctctgtcagccagcaggacagtgcattctccatactatcgtggtagattcgcatcagattcttcgtcattgtggaacgggcatatcgccgtgctgtgctgtctgaggccatgcacagactgttctcaggcagtagcggcgactgccgatcagaccatagcgatccatcttgagcgacctgttgtacactattagtatcggtaaccgagtccggatccgtagtctgctcttccagctcgcgaatgaggtcatccgtgttcgggctatggggcggaagaaggactgcttgaaaactgagcggactgagaggtttctcgagctgttggcccatgctgaaatcaccggggatggctagatcccacggaaaaggtgcgtctgactgcacgttggccgcatcttctgcatttgcgggaatagcagagtggctgaaatcgaaaaggtcgccggggtgggagagtagcccctgagtccagctcgggagcgcgtcgtgagagtttatgactggaggcgcatcgtgattgtcactttccggtgtagggattgttgcagctgaagttgatggttcactggtggttgttgcggtcctggctttctttgttctcgctctaggtgcagccccttttgccttggagcgttgggatgagagccaattgaaggtacaatccttgttccaacgcttgcaatttgaacacgaaacccagccgttttcattggcctcgtttctattttccggggcatcacagcgtcgcttgcccttgcgacagggatcgcagctatgattctggcgtcggcgcgtatctgccatgctgtgtgcaggagaatatcgacagtgatatctgtatagatggt

SEQ ID NO: 35 - DNA, 260 bp, Aspergillus nidulans
atgcatatgcgggatagttccgacctaggattggatgcatgcggaaccgcacgagggcggggcggaaattgacacaccactcctctccacgcaccgttcaagaggtacgcgtatagagccgtatagagcagagacggagcactttctggtactgtccgcacgggatgtccgcacggagagccacaaacgagcggggccccgtacgtgctctcctaccccaggatcgcatccccgcatagctgaacatctatataactgca

SEQ ID NO: 36 - DNA, 20 bp, Artificial Sequence
  A synthetic oligonucleotide
cgtggaccgtcgttcttatt

SEQ ID NO: 37 - DNA, 20 bp, Artificial Sequence
  A synthetic oligonucleotide
atactttctcggcaggagca

SEQ ID NO: 38 - DNA, 20 bp, Artificial Sequence
  A synthetic oligonucleotide
tgtggaattgtgagcggata

SEQ ID NO: 39 - DNA, 20 bp, Artificial Sequence
  A synthetic oligonucleotide
cgcgtcgtgagagtttatga

SEQ ID NO: 40 - DNA, 20 bp, Artificial Sequence
  A synthetic oligonucleotide
cgtggaccgtcgttcttatt

SEQ ID NO: 41 - DNA, 20 bp, Artificial Sequence
  A synthetic oligonucleotide
atactttctcggcaggagca

SEQ ID NO: 42 - DNA, 20 bp, Artificial Sequence
  A synthetic oligonucleotide
ttcctacactgggtcccaac

SEQ ID NO: 43 - DNA, 20 bp, Artificial Sequence
  A synthetic oligonucleotide
gaagcaagccttgaatcgtc

SEQ ID NO: 44 - DNA, 20 bp, Artificial Sequence
  A synthetic oligonucleotide
cgtggaccgtcgttcttatt

SEQ ID NO: 45 - DNA, 20 bp, Artificial Sequence
  A synthetic oligonucleotide
atactttctcggcaggagca

SEQ ID NO: 46 - DNA, 21 bp, Artificial Sequence
  A synthetic oligonucleotide
aatcttcccagtcccaactgt

SEQ ID NO: 47 - DNA, 20 bp, Artificial Sequence
  A synthetic oligonucleotide
gaagcaagccttgaatcgtc

SEQ ID NO: 48 - DNA, 20 bp, Artificial Sequence
  A synthetic oligonucleotide
gatgtgaaggaatcgcccta

SEQ ID NO: 49 - DNA, 20 bp, Artificial Sequence
  A synthetic oligonucleotide
atactttctcggcaggagca

SEQ ID NO: 50 - DNA, 20 bp, Artificial Sequence
  A synthetic oligonucleotide
gatgtgaaggaatcgcccta

SEQ ID NO: 51 - DNA, 20 bp, Artificial Sequence
  A synthetic oligonucleotide
atactttctcggcaggagca

SEQ ID NO: 52 - DNA, 20 bp, Artificial Sequence
  A synthetic oligonucleotide
ttcctacactgggtcccaac

SEQ ID NO: 53 - DNA, 20 bp, Artificial Sequence
  A synthetic oligonucleotide
ccgagcaaagtgagtgaaca

SEQ ID NO: 54 - DNA, 20 bp, Artificial Sequence
  A synthetic oligonucleotide
gatgtgaaggaatcgcccta

SEQ ID NO: 55 - DNA, 20 bp, Artificial Sequence
  A synthetic oligonucleotide
atactttctcggcaggagca

SEQ ID NO: 56 - DNA, 21 bp, Artificial Sequence
  A synthetic oligonucleotide
aatcttcccagtcccaactgt

SEQ ID NO: 57 - DNA, 20 bp, Artificial Sequence
  A synthetic oligonucleotide
ccgagcaaagtgagtgaaca

SEQ ID NO: 58 - DNA, 20 bp, Artificial Sequence
  A synthetic oligonucleotide
gcgttcaaaagtcgcctaag

SEQ ID NO: 59 - DNA, 20 bp, Artificial Sequence
  A synthetic oligonucleotide
tggaccacacggtaatagca

SEQ ID NO: 60 - DNA, 20 bp, Artificial Sequence
  A synthetic oligonucleotide
gcgttcaaaagtcgcctaag

SEQ ID NO: 61 - DNA, 20 bp, Artificial Sequence
  A synthetic oligonucleotide
tggaccacacggtaatagca

SEQ ID NO: 62 - DNA, 20 bp, Artificial Sequence
  A synthetic oligonucleotide
gcgttcaaaagtcgcctaag

SEQ ID NO: 63 - DNA, 20 bp, Artificial Sequence
  A synthetic oligonucleotide
atgaaggagacaagcgttgg

SEQ ID NO: 64 - DNA, 21 bp, Arabidopsis thaliana
caaaggccaacagagagaaga

SEQ ID NO: 65 - DNA, 20 bp, Arabidopsis thaliana
atcaccagaatccagcacaa

SEQ ID NO: 66 - DNA, 20 bp, Arabidopsis thaliana
gcggtgaacgataaattgct

SEQ ID NO: 67 - DNA, 20 bp, Arabidopsis thaliana
gcttgctcccatctctgttc

SEQ ID NO: 68 - DNA, 20 bp, Arabidopsis thaliana
gctaatatcgcctccgatca

SEQ ID NO: 69 - DNA, 20 bp, Arabidopsis thaliana
cttcatcctttgcaacacca

SEQ ID NO: 70 - DNA, 20 bp, Arabidopsis thaliana
ggtgatgaagcacaatccaa

SEQ ID NO: 71 - DNA, 20 bp, Arabidopsis thaliana
cagtaaggtcacgtccagca

SEQ ID NO: 72 - DNA, 20 bp, Arabidopsis thaliana
tgccatttggtgtttttgag

SEQ ID NO: 73 - DNA, 20 bp, Arabidopsis thaliana
gaccgggatatgctcctaca

SEQ ID NO: 74 - DNA, 20 bp, Arabidopsis thaliana
attcgggtgcattcaaagag

SEQ ID NO: 75 - DNA, 20 bp, Arabidopsis thaliana
ggcttggatgctgcttatgt

SEQ ID NO: 76 - DNA, 20 bp, Zea mays
ccgaaaatgcttctgagctt

SEQ ID NO: 77 - DNA, 20 bp, Zea mays
gcatgcgcttagttgggtat

SEQ ID NO: 78 - DNA, 20 bp, Zea mays
tggtcgtgctatgagctttg

SEQ ID NO: 79 - DNA, 20 bp, Zea mays
caaagctcatagcacgacca

SEQ ID NO: 80 - DNA, 20 bp, Zea mays
ctgtacgggaacatcgtcct

SEQ ID NO: 81 - DNA, 21 bp, Zea mays
gcatctgaatcacgaagcagg

SEQ ID NO: 82 - DNA, 20 bp, Zea mays
atgaaggagacaagcgttgg

SEQ ID NO: 83 - DNA, 20 bp, Zea mays
ctgctccagcataaccttcc

SEQ ID NO: 84 - DNA, 2006 bp, Arabidopsis thaliana
gagacatgtgagcttcatctactactcatattgcttatattttcctacaacactctctcaacaaaaaaacaaaaaaaaaagacgttctttctttgtttgtatttagaagaaaaatggctaccaattacaacgagaagcttcttcttaattatgttcccgtttacgttatgcttccgttgggagttgtgaatgtggaaaatgtatttgcggacccagaaacgcttgaaacgcagcttaaacgtctcaaagaagaagctggcgttgatggcgttatggtcgatgtttggtggggaatcatagaatccaaaggtcccaaacaatatgattggacggcctacaaaacgctgttccagctgatcgcacgtttgggactcaaaatccaagcaatcatgtcttttcaccaatgtggtggaaacgttggcgacatcgttactatcccgatcccacaatgggttcgcgatgtcggtgacaatgatcccgatatctactacactaaccgtaaaggaactagagacatcgagtatctctcaatcggtgttgataatcttcccctatttgccggaagaaccgctgttcagttgtacagtgattacatgagtagcttcaaagaaaacatggcggatttgatagaagctggggtgattgttgacatcgaagtcggacttggcccggccggtgaactacgttatccttcttacccccaaagccaaggttgggtgtttcccggcatcggagaattccagtgttatgacaagtacttgaagaaagatttcaaggaagcggcggcgaaagcagggcaccctgagtgggacttgccagaggacgccggagaatacaatgacaagccggaggagactggatttttcaagaaggatgggacttatgtctcggagaaggggaagtttttcatgacatggtactcgaacaaactaatttttcatggagatcagatcttaggagaagccaacaagatctttgctggacttaaagttaacttggctgccaaggtttctgggattcactggttgtacaaccaccacagccacgctgcggagttgactgctggatattacaaccttttcaagagagatggttaccgtccgatcgcccggatgctctcaaaacactacggcattctcaacttcacttgccttgagatgaaagataccgacaatacagctgaagccctaagtgctcctcaagaacttgttcaagaggtactgagcaaggcatggaaagaaggtatagaagttgcgggtgagaacgcattagagacctatggagccaaaggttacaatcagattcttcttaacgcgaggcctaacggggttaacccaaacggtaagccgaagcttagaatgtacggatttacttaccttcggttatccgatactgtctttcaagaaaacaactttgagctgtttaagaagttggtgaggaaaatgcacgctgatcaagattattgtggagacgcagcgaagtacgggcatgagattgtgccgttgaaaacgtcgaattcgcagctgacgctggaggatatcgccgacgcggctcagccaagtggagcatttaagtgggactctgaaaccgatttgaaggtcgacggttagttatattttatatgatcgatcgtctaatcacaaagaaagtaagggaaagaatcaaggaagtaagaataagaagataataaataaagagaataagaagaggcatttgcaactttaagaggttaaaatttcttaaatgagtctgagtttcgttgtgtgttttgggtgtaccataagaaataaaacgtacggggttgctatgtattttgtgaagtgtgtatttctggttgaatattagtctgcgtaataaaaaaaagtatgtgtagtaaaaataaataactgttgccagtaaaataatgcatcattgtcactgtattgccagaatatatatattactttttaaggttgtaaagaatttaagattatatatttttaatggctaatttgaagttggatatt